SEQUENCE LISTING (30 sequences)

SEQ ID NO: 1 - protein, 310 aa, unidentified
  source 1..310: note = Saccharomyces paradoxus
MPVTIKSRSKALRDTEIDLSKKPTLDDVLKQISTNNRNISKYRIRLTYKKETKQVPVISESFFREEADDSMEFFIKDLGPQISWRLVFFCEYLGPVLVHSLFYYLSTIPTVVDKWHSSSSEYNPFLNKVAYFLILGHYGKRLFETLFVHQFSLATMPIFNLFKNCFHYWVLSGLISFGYFGYGFPFGNAKLFKYYSYLKLDDLSTLIGLFVLSELWNFYCHIKLRLWGDYQKKHGNAKVRVPLNQGIFNLFVAPNYTFEVWSWIWFTFVFKFNLFAVLFLTVSTVQMYAWAQKKNKKYHTRRAFLIPFVF

SEQ ID NO: 2 - DNA, 954 bp, synthetic construct
  source 1..954: mol_type = other DNA
tattttgaatttaatttgaaaatgccagttactatcaaaagcagatctaaggcgttaagagatactgagatcgatttgtcaaagaagcccacattggacgatgtactgaaacagatatccaccaacaaccgtaacatttctaagtatcgtataaggttgacatataagaaggagaccaagcaggttcctgttatttcagaatctttcttcagagaggaagctgatgattctatggaattcttcattaaggatctaggaccccaaatatcttggagattagtcttcttttgcgaatatctaggtcctgtcttagtgcattcactattttactacctaagtactattcccactgtggttgacaaatggcattcttctagcagtgaatacaacccattcttaaataaagtagcttactttctgatacttggtcactacggaaagaggctattcgaaaccctgttcgttcaccagttttccttagccactatgccaatattcaatttatttaagaattgtttccactactgggttctatctggtcttatttcgttcggctactttggatacgggtttcctttcggtaatgctaaattgttcaaatactattcatacctgaaattggacgacctttccacactgataggtcttttcgtgttgtctgaattgtggaatttctactgtcatatcaaattacgtttatggggtgactaccagaagaaacacgggaatgcaaaagttagagtgcctctgaaccaaggcatcttcaatttatttgtcgctcctaattacactttcgaggtatggtcttggatttggtttacattcgtatttaagtttaatctgttcgctgtgttgtttctgacagtatcaacggtacagatgtatgcttgggctcagaagaagaacaagaaataccataccaggagagcattcttgatcccattcgtcttctag

SEQ ID NO: 3 - protein, 310 aa, unidentified
  source 1..310: note = Saccharomyces aroboricola
MTVTIKSRSKGLRDTEVDLSGKPTLDDVLKQISSNNRNISKYRIRLTYKKEDKQVPVISDTFFQEKADDSMEFFIKDLGPQISWRLVFFCEYLGPILVHSLFYYLSTIPKIVEKCHSSSSEYNPFLNRVGYSLIVAHYGKRLFETLFIHQFSLATMPIFNLFKNCFHYWVLSGLISFGYFGYGFPFGNAKLFRYYSYLKLDDLGTLIGLFVVSELWNLYCHIKLRLWGDYQKKHGNSKVRVPLNQGIFKLFVAPNYTFEVWSWVWFTFVFKLNLFAVLFLTVSAVQMYAWAQKKNKKYHTRRAFLIPYIF

SEQ ID NO: 4 - DNA, 933 bp, synthetic construct
  source 1..933: mol_type = other DNA
atgacagttacgataaagagtcgtagcaagggacttagggataccgaagtggatttatctggtaagccgacgcttgacgacgtgttaaagcaaatatcatcaaataatcgtaatatatccaaatacagaattaggttgacatataagaaggaggataaacaggtcccggtcatttctgacacgttcttccaagagaaggcagatgattctatggagttcttcatcaaagacctgggaccccaaatatcatggagactggtcttcttttgtgaatacttgggacccatattggtgcattcccttttttattacctaagcacaataccgaagattgtcgagaaatgccattcatcttctagcgaatacaatcccttcctaaatagggttggatatagtcttatcgtggcgcattacgggaaaaggctgttcgaaactcttttcatccaccaatttagcctggcaactatgcctatattcaacctattcaaaaattgcttccactactgggtactgagtggtctgatttcctttgggtattttggctacggcttcccttttgggaatgcgaaacttttcagatactacagctacttaaaactagatgacttaggtacactaataggtttgttcgtagtctctgaattatggaatctttattgtcacataaaattacgtttgtggggtgattatcagaagaaacacggtaactcaaaggtccgtgtgccgttgaatcaaggaatcttcaagcttttcgtggctcctaactacacgtttgaagtctggtcatgggtgtggttcacttttgtttttaagctaaatttatttgcggtcctgttccttactgtatccgcagtacagatgtatgcgtgggctcaaaagaaaaacaaaaagtatcataccagacgtgcctttttgattccctacatcttttaa

SEQ ID NO: 5 - protein, 310 aa, unidentified
  source 1..310: note = Kazachstania barnettii
MVAVKSRSKSLRDTEIAINKNSTLESVLIAISKNNKGISKYRLRVTYLKESKQVPITEESFFNEGNNKDIELFVKDLGPQISWRLVFVIEYLGPLLVHSLVYYLSLNPKFMAKFGTSKINNVDPALNRIAFFMVMGHYLKREIETLFVHKFTLATMPLFNVFKNSFHYWILNGIIALGYLGYGFVVPNCCYEKTLNYVGLNNLNTLVSLFVLSEAWNAYIHIRLRIFGDQQRKIGNNTKRVPISTGIFKIFVAPNYTFEVWSWIWFALAFKLNLFAVFFVTVSATQMYLWAMKKNRRYGTKRSFIIPFIF

SEQ ID NO: 6 - DNA, 933 bp, synthetic construct
  source 1..933: mol_type = other DNA
atggttgcagtaaagtctagaagtaagtctttaagagataccgaaattgccataaataagaattctactcttgaaagtgtgttgatcgcaatttcaaagaacaataaaggtatttcaaagtacagattgagagtcacatatctgaaggagtctaaacaagttccaataactgaagaaagtttcttcaatgaagggaataacaaggatatcgaactcttcgttaaagatttaggtccgcaaatatcgtggagacttgtgtttgttattgaatatctcggtcctttattggtacattctctagtttattatctgagtttaaacccaaagtttatggctaaatttggcacaagtaagattaataatgtagatcctgcattgaatagaatcgccttcttcatggtaatgggccactatttaaagagagaaatcgaaactttgttcgttcataaatttactcttgctacaatgccattatttaatgtgtttaagaatagctttcattactggatcttaaatggcatcattgcattgggttatctgggatatgggttcgtggttcccaattgttgttatgagaagactttgaactatgtaggccttaataatttgaataccttagtttcgttgtttgtattgtctgaggcttggaatgcgtatatccacatccgattgcggatcttcggtgatcaacaaagaaagatagggaataatacgaagagagttccaatttcgacaggaatcttcaagatcttcgttgctcccaattatacgtttgaagtttggtcttggatttggttcgctttggccttcaagctcaacctattcgcagtattcttcgtgactgtgtccgctacacaaatgtacctatgggccatgaagaagaaccgtagatatggtactaagcgtagctttattataccctttatattctaa

SEQ ID NO: 7 - protein, 327 aa, unidentified
  source 1..327: note = Yarrowia lipolitica
MVNLSLRPRPAKAKFKGLPANLEVSPEDTVASVVAKLSAATKLSKSRIRLTVADEENGGAPGAKKKHIVLKPEHAVGDYLFSDSPVVFVKDLGPQIPWRTVFILEYLGPLLAHPIIFFGQKFFYRQSFEYTFAQKLVFTLCMLHFLKREIETIYIHKFSSATMPLFNLFKNSGYYWFIAGFNLAFFVYAPASFSSPQAPLWKRFLFSTGFFERTPLFLNLMATLWLWGETSNFWTHFNLASLRNDGSKDHKIPFGYGFNLVSCPNYFFEVVSWIAIALMCGNWSAYVFTAIGFGQMYVWAVQKHRRYKREFGDRYPRNRKVMVPFLL

SEQ ID NO: 8 - DNA, 984 bp, synthetic construct
  source 1..984: mol_type = other DNA
atggtcaatctgtctctgcgtccccgtcctgccaaggccaagttcaagggtctgcccgccaacctcgaggtgtctcctgaggacacggtggcctctgtggtggcgaaattgagcgctgccaccaaactgtccaagtcccgaattcggctcactgtggccgatgaggagaatggaggcgctcccggagccaagaagaagcacattgtgctcaagcccgagcacgctgtcggcgattacctcttctccgactctcctgtggtgtttgtgaaggacttgggcccccagatcccctggagaaccgtcttcattctcgagtacctgggacctcttctggcccaccccatcatcttctttggccagaagttcttttaccgacagagctttgagtacacgtttgctcagaagctggtcttcactctatgcatgctgcatttcctcaagcgagagattgagaccatctacatccacaagttctcctccgccaccatgcctctgttcaacctctttaagaactccggctactactggttcattgccggtttcaaccttgcctttttcgtttatgcccccgcctccttttcttctccccaggctcctctgtggaagcgtttcctcttttccactggcttctttgagcgaacccctctgttcctcaacctcatggccaccctgtggctctggggagagacctccaacttctggacccacttcaacttggcttctctgagaaacgatggctccaaggaccacaagatcccctttggctacggcttcaaccttgtttcctgccccaactacttctttgaggttgtcagctggatcgcaattgccctcatgtgtggcaactggtccgcttatgtctttactgccattggcttcggtcagatgtacgtctgggccgtccagaagcaccgacgatacaagcgggagtttggagaccggtaccctcgaaacagaaaggtcatggttcctttcttgctttag

SEQ ID NO: 9 - protein, 310 aa, Saccharomyces cerevisiae
MPITIKSRSKGLRDTEIDLSKKPTLDDVLKKISANNHNISKYRIRLTYKKESKQVPVISESFFQEEADDSMEFFIKDLGPQISWRLVFFCEYLGPVLVHSLFYYLSTIPTVVDRWHSASSDYNPFLNRVAYFLILGHYGKRLFETLFVHQFSLATMPIFNLFKNCFHYWVLSGLISFGYFGYGFPFGNAKLFKYYSYLKLDDLSTLIGLFVLSELWNFYCHIKLRLWGDYQKKHGNAKIRVPLNQGIFNLFVAPNYTFEVWSWIWFTFVFKFNLFAVLFLTVSTAQMYAWAQKKNKKYHTRRAFLIPFVF

SEQ ID NO: 10 - DNA, 933 bp, synthetic construct
  source 1..933: mol_type = other DNA
atgcctatcaccataaaaagccgctctaaagggttaagggacactgaaattgacttatccaaaaagcctactttagatgatgttttgaaaaaaatctctgctaataaccacaatatcagcaagtacaggataagattaacctacaaaaaggaatctaaacaagttccggttatttcagaatcgttttttcaagaagaggctgatgactcaatggaattcttcatcaaagatttgggtccccaaatttcatggagattagtcttcttttgtgagtatttgggtccagtcttggttcactcccttttttattatctatctaccattcccacagttgttgatagatggcacagtgctagctccgactataatccatttttaaacagggttgcatattttttaattttaggacattatggaaagagattatttgaaaccttatttgttcaccaattctctttagctactatgccaattttcaacctgttcaaaaattgtttccattactgggttctaagcggtctcatttcattcggttactttggctacggcttcccctttgggaatgctaagttattcaaatactattcatatttgaaattggatgacttgagtacattaattggtcttttcgtgctttcagaactatggaacttttattgccacattaaattgcgcctatggggtgactatcaaaagaagcatggtaacgctaagatccgtgtcccattgaatcaaggtattttcaatctttttgttgctcccaactatacttttgaagtttggtcttggatttggtttacttttgtgttcaagttcaatttatttgccgttttatttttgactgtttcaacagctcaaatgtacgcatgggctcaaaagaaaaacaaaaagtatcataccagaagagcattcttgattccatttgtattttga

SEQ ID NO: 11 - protein, 561 aa, Arabidopsis thaliana
MAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLWELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGL

SEQ ID NO: 12 - DNA, 1686 bp, synthetic construct
  source 1..1686: mol_type = other DNA
atggcgccacaagaacaagcagtttctcaggtgatggagaaacagagcaacaacaacaacagtgacgtcattttccgatcaaagttaccggatatttacatcccgaaccacctatctctccacgactacatcttccaaaacatctccgaattcgccactaagccttgcctaatcaacggaccaaccggccacgtgtacacttactccgacgtccacgtcatctcccgccaaatcgccgccaattttcacaaactcggcgttaaccaaaacgacgtcgtcatgctcctcctcccaaactgtcccgaattcgtcctctctttcctcgccgcctccttccgcggcgcaaccgccaccgccgcaaaccctttcttcactccggcggagatagctaaacaagccaaagcctccaacaccaaactcataatcaccgaagctcgttacgtcgacaaaatcaaaccacttcaaaacgacgacggagtagtcatcgtctgcatcgacgacaacgaatccgtgccaatccctgaaggctgcctccgcttcaccgagttgactcagtcgacaaccgaggcatcagaagtcatcgactcggtggagatttcaccggacgacgtggtggcactaccttactcctctggcacgacgggattaccaaaaggagtgatgctgactcacaagggactagtcacgagcgttgctcagcaagtcgacggcgagaacccgaatctttatttccacagcgatgacgtcatactctgtgttttgcccatgtttcatatctacgctttgaactcgatcatgttgtgtggtcttagagttggtgcggcgattctgataatgccgaagtttgagatcaatctgctatgggagctgatccagaggtgtaaagtgacggtggctccgatggttccgccgattgtgttggccattgcgaagtcttcggaaacggagaagtatgatttgagctcgataagagtggtgaaatctggtgctgctcctcttggtaaagaacttgaagatgccgttaatgccaagtttcctaatgccaaactcggtcagggatacggaatgacggaagcaggtccagtgctagcaatgtcgttaggttttgcaaaggaaccttttccggttaagtcaggagcttgtggtactgttgtaagaaatgctgagatgaaaatagttgatccagacaccggagattctctttcgaggaatcaacccggtgagatttgtattcgtggtcaccagatcatgaaaggttacctcaacaatccggcagctacagcagaaaccattgataaagacggttggcttcatactggagatattggattgatcgatgacgatgacgagcttttcatcgttgatcgattgaaagaacttatcaagtataaaggttttcaggtagctccggctgagctagaggctttgctcatcggtcatcctgacattactgatgttgctgttgtcgcaatgaaagaagaagcagctggtgaagttcctgttgcatttgtggtgaaatcgaaggattcggagttatcagaagatgatgtgaagcaattcgtgtcgaaacaggttgtgttttacaagagaatcaacaaagtgttcttcactgaatccattcctaaagctccatcagggaagatattgaggaaagatctgagggcaaaactagcaaatggattgtga

SEQ ID NO: 13 - protein, 556 aa, Arabidopsis thaliana
MTTQDVIVNDQNDQKQCSNDVIFRSRLPDIYIPNHLPLHDYIFENISEFAAKPCLINGPTGEVYTYADVHVTSRKLAAGLHNLGVKQHDVVMILLPNSPEVVLTFLAASFIGAITTSANPFFTPAEISKQAKASAAKLIVTQSRYVDKIKNLQNDGVLIVTTDSDAIPENCLRFSELTQSEEPRVDSIPEKISPEDVVALPFSSGTTGLPKGVMLTHKGLVTSVAQQVDGENPNLYFNRDDVILCVLPMFHIYALNSIMLCSLRVGATILIMPKFEITLLLEQIQRCKVTVAMVVPPIVLAIAKSPETEKYDLSSVRMVKSGAAPLGKELEDAISAKFPNAKLGQGYGMTEAGPVLAMSLGFAKEPFPVKSGACGTVVRNAEMKILDPDTGDSLPRNKPGEICIRGNQIMKGYLNDPLATASTIDKDGWLHTGDVGFIDDDDELFIVDRLKELIKYKGFQVAPAELESLLIGHPEINDVAVVAMKEEDAGEVPVAFVVRSKDSNISEDEIKQFVSKQVVFYKRINKVFFTDSIPKAPSGKILRKDLRARLANGLMN

SEQ ID NO: 14 - DNA, 1671 bp, synthetic construct
  source 1..1671: mol_type = other DNA
atgactacgcaggatgttattgtcaatgatcaaaatgaccaaaagcaatgttcgaatgatgttatctttcgtagtagactccctgatatatacatacctaaccatctaccattgcatgattacatatttgaaaatatatcggaatttgctgctaagccatgcctaatcaatggtccaacaggtgaagtgtatacctatgctgatgttcatgttacttccaggaagctcgctgctggtttgcacaacttgggcgttaaacagcatgacgtcgttatgatattgctgccaaatagcccagaagtggtacttactttcttggccgcctcgtttattggcgccattacgacatccgcaaatcccttcttcacgcccgctgaaatttctaaacaagctaaagcatctgctgctaaattaatcgtcacacaaagtagatatgttgataagattaagaacttacaaaacgatggggtcttaattgtcacaaccgattctgatgctatccctgaaaattgtctgagattctctgagttaactcaatccgaagagcctagagtagacagtatacctgagaagatctctccagaagatgtggtggctttgccattttcctcaggtactaccggtctgccaaagggtgtgatgttgactcacaagggtttggtgacgtcagtagctcagcaagtagatggggagaaccctaatctgtatttcaatagagatgacgtcattttgtgcgtattacctatgttccatatttatgcattaaactcgattatgctatgctctctgcgagttggagcaactatattaatcatgccaaagtttgagataactctcttgttagaacaaattcagaggtgcaaggtcactgttgctatggtagtaccaccaatagtcctggcaatcgcaaagagtcctgaaaccgagaagtatgatttaagtagtgtgcggatggttaaatcaggcgctgcccctctaggtaaagaattagaagatgccatttccgctaaatttccgaatgcaaaattaggccaaggatatggcatgacggaagctggtccagttctagcaatgtctttggggtttgctaaagagccttttcccgtaaagagcggtgcctgtggcactgttgtgcgtaatgctgagatgaaaatactggatccagacacgggcgattcactaccacgcaataaaccaggcgagatatgtataaggggaaaccagattatgaaggggtatttgaacgatcccctggccaccgcctcaactatcgataaggacggatggttacacactggtgacgttgggtttattgacgatgatgatgaattattcatcgttgacagattaaaggaattgatcaaatacaaaggttttcaagtagctccagcagaactcgaaagccttttgattggacatccagagataaatgacgtcgcagtggtcgctatgaaagaagaggatgctggtgaagttcccgttgcatttgtagttagatcgaaggattccaacattagcgaggacgaaattaaacaatttgtaagcaaacaggttgtcttttataaaagaatcaataaagttttcttcactgactcaattccaaaggccccttctggtaaaatcctgcgtaaggacttgagggcacgattggctaatggcctcatgaattga

SEQ ID NO: 15 - protein, 717 aa, Arabidopsis thaliana
MDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGDLVPLSYIAGLLTGRPNSKATGPDGESLTAKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 16 - DNA, 2154 bp, synthetic construct
  source 1..2154: mol_type = other DNA
ctaacaaatcggtattggagcaccgttccattcctttaagcaatccatcaacgggtctattagtttaccctcacacattgcagtaaagactttatcaaattcttccccgggagaaactaccttttcacctgtaagtaactttgtgccaagctcttctctaacaaatctgtataaggggtaggatctacactcctttatacgattaggtattggggcagttccgttaccataggcagctcttgcagcttccacctcttttggtagtacggcttttagctcttcttcaaatgcaccaattttctggaaaatagaagtgacggcattcttctcggtttctccatttgataaggcatggtctacaattacttgacgtaatctctgcattaagggatacgtagctgaacagggatcatcaacataagtgaaaacttgttccctgtcaacaacctttagcaaatccttttcacaaaacctggaggggtgtaattcgccgttaattccggttgttaagaccttcttggcaacctgacttacagtattcttaacagtctgtctcaggttctcttcaagatgtctaaggtcaacagcctggcagattccaactagaaatgttgtagacatcaacttgagtatgtctactgcttcggaagtctttctcgatgaaattaaaccgagagagttcacgtcctgattatgttgttctgcggattgcacatgagatgttacaggattggctaaatattggagctccgaacagtaacttgccatggctatttctgctcctttgaaaccgtaatctaaggatgggttactggaagcggttaaattggacggtagtccattgttatagaaatcgttcaccaattctgaaaattgagcgaacattaatttaccaatcgccgcgatagctagtctcgtgttgtccattgacacaccaataggtgtcccttggaagttaccaccatgaatggctttattgcggctgacgtcaattagaggattatcattcacggaattaatctccctttcgatgctcttagtggcttgtcttattacttcaatttgtgggcccagccattgtggtgaggttctcaaagcatacctatcttgtttaggcttttgtagtggatccatttcatgcaccttttgagcgagtttcatataggagctcccatccaaaatgtgttccattatagcagctgcctcaatttgacctggatgatgcttcaatctatgagtcaaatgatcagtaaactctggttttcctgacatcacttccgcaaatatagccgacagcacctcagccaacactgcttgaacattggcctcgaaaagaaccatactggccatacccgaaccaactgctgtcccattaacaagcgctaggccttccttgggttgaagatcaaagaatccagtcgatatgcctgccttttcgaatgcctcctttgccgtaagagattccccatctgggccggttgctttagaattaggtctacctgttaataatcctgcaatataagacagtggtacaagatcaccactggccgttatagtgccccttaatggtagtgaagggctgatattatggttcagaagggacgttatagcttcaaggatttcgaacctaatacccgaataaccctgcaaaagagtatttacccttaccaacattgcagctctggtagctgattgaggtaaagtatgacatgtctctttggtattgccaaagattccagcgttcaagaagcggattagttcagtttgaagagcagtaccattctttgtcctcctatgcgaggttgccccgaaaccggtagtcacaccgtatgaatccgtgcctttgttcatactttccattacccaatctgaagaagcttttacgccagccctagatgtttccgctaattcaactttgacgctgccaccaacggtactaatagccgctacttgtcctatcgtcaaagtttcacctcctagattcaccacgggcctacggtactcttcgaccattttcttaacttcatcaagatggcttcctttcatctggtctgcggccagtccccagtttaaaggatcagccaatgtcttagtagtaacggccactttagtcttttcgccacctccacagagcattgcttcgatttgatccat

SEQ ID NO: 17 - protein, 505 aa, Arabidopsis thaliana
MDLLLLEKSLIAVFVAVILATVISKLRGKKLKLPPGPIPIPIFGNWLQVGDDLNHRNLVDYAKKFGDLFLLRMGQRNLVVVSSPDLTKEVLLTQGVEFGSRTRNVVFDIFTGKGQDMVFTVYGEHWRKMRRIMTVPFFTNKVVQQNREGWEFEAASVVEDVKKNPDSATKGIVLRKRLQLMMYNNMFRIMFDRRFESEDDPLFLRLKALNGERSRLAQSFEYNYGDFIPILRPFLRGYLKICQDVKDRRIALFKKYFVDERKQIASSKPTGSEGLKCAIDHILEAEQKGEINEDNVLYIVENINVAAIETTLWSIEWGIAELVNHPEIQSKLRNELDTVLGPGVQVTEPDLHKLPYLQAVVKETLRLRMAIPLLVPHMNLHDAKLAGYDIPAESKILVNAWWLANNPNSWKKPEEFRPERFFEEESHVEANGNDFRYVPFGVGRRSCPGIILALPILGITIGRMVQNFELLPPPGQSKVDTSEKGGQFSLHILNHSIIVMKPRNC

SEQ ID NO: 18 - DNA, 1518 bp, synthetic construct
  source 1..1518: mol_type = other DNA
atggacttgttgttgttggaaaagtctttgatcgctgttttcgttgctgttatcttggctactgttatctctaagttgagaggtaagaagttgaagttgccaccaggtccaatcccaatcccaatcttcggtaactggttgcaagttggtgacgacttgaaccacagaaacttggttgactacgctaagaagttcggtgacttgttcttgttgagaatgggtcaaagaaacttggttgttgtttcttctccagacttgactaaggaagttttgttgactcaaggtgttgaatttggttcaagaactagaaacgttgttttcgacatcttcactggtaagggtcaagacatggttttcactgtttacggtgaacactggagaaagatgagaagaatcatgactgttccattcttcactaacaaggttgttcaacaaaacagagaaggttgggaatttgaagctgcttctgttgttgaagatgttaagaagaacccagactctgctactaagggtatcgttttgagaaagagattgcaattgatgatgtacaacaacatgttcagaatcatgttcgacagaagattcgaatctgaagatgacccattgttcttgagattgaaggctttgaacggtgaaagatcaagattggctcaatctttcgaatacaactacggtgacttcatcccaatcttaagaccattcttgagaggttacttgaagatctgtcaagacgttaaggacagaagaatcgctttgttcaagaagtacttcgttgacgaaagaaagcaaatcgcttcttctaagccaactggttctgaaggtttgaagtgtgctatcgaccacatcttggaagctgaacaaaagggtgaaatcaacgaagataacgttttgtacatcgttgaaaacatcaacgttgctgctatcgaaactactttgtggtctatcgaatggggtatcgctgaattggttaaccacccagaaatccaatctaagttgagaaacgaattggacactgttttgggtccaggtgttcaagttactgaaccagacttgcacaagttgccatacttgcaagctgttgttaaggaaactttgagattgagaatggctatcccattgttggttccacacatgaacttgcacgacgctaagttggctggttacgacatcccagctgaatctaagatcttggttaacgcttggtggttggctaacaacccaaactcttggaagaagccagaagaatttagaccagaaagattcttcgaagaagaatctcacgttgaagctaacggtaacgacttcagatacgttccattcggtgttggtagaagatcttgtccaggtatcatcttggctttgccaatcttgggtatcactatcggtagaatggttcaaaacttcgaattgttgccaccaccaggtcaatctaaggttgacacttctgaaaagggtggtcaattctctttgcacatcttgaaccactctatcatcgttatgaagccaagaaactgttaa

SEQ ID NO: 19 - protein, 392 aa, Vitis vinifera
MASVEEFRNAQRAKGPATILAIGTATPDHCVYQSDYADYYFKVTKSEHMTALKKKFNRICDKSMIKKRYIHLTEEMLEEHPNIGAYMAPSLNIRQEIITAEVPKLGKEAALKALKEWGQPKSKITHLVFCTTSGVEMPGADYKLANLLGLEPSVRRVMLYHQGCYAGGTVLRTAKDLAENNAGARVLVVCSEITVVTFRGPSEDALDSLVGQALFGDGSAAVIVGSDPDISIERPLFQLVSAAQTFIPNSAGAIAGNLREVGLTFHLWPNVPTLISENVEKCLTQAFDPLGISDWNSLFWIAHPGGPAILDAVEAKLNLDKKKLEATRHVLSEYGNMSSACVLFILDEMRKKSLKGERATTGEGLDWGVLFGFGPGLTIETVVLHSIPMVTN

SEQ ID NO: 20 - DNA, 1179 bp, synthetic construct
  source 1..1179: mol_type = other DNA
atggcttctgttgaggaatttaggaatgctcaacgtgccaagggacccgccactattctggctataggtactgccaccccagatcattgcgtatatcaatcggattacgctgactactacttcaaggttaccaaaagtgagcacatgacagccttgaagaagaagtttaaccgtatatgcgataagtcaatgatcaagaaaagatacattcacttgacagaagaaatgttagaggaacatccaaatataggcgcttacatggctccatcgttaaacatccgtcaggaaatcattacagctgaagtacccaaattaggtaaagaggctgcattgaaagccctaaaagaatggggccaacctaaatccaaaattactcatttggtattctgtaccacaagcggcgttgaaatgcctggagctgactataaacttgccaacctactgggcttggaaccttccgtccgtagggtaatgctttaccaccaaggttgttatgctggtgggacagtcttgaggacggctaaggacttagccgaaaataatgctggggcacgggttctagttgtatgttcggaaattacggttgtaacttttcgtggtccatcagaagatgcattagattcgttggtcggtcaggcattatttggcgatggctccgcagcagtcatcgtcggttcggatccagatattagtatagagcgccccttgttccaactcgtatccgcagctcaaacatttattccaaactccgcgggtgcgattgccgggaacttacgggaagtgggtttaacctttcacctctggccaaatgttcctacccttatttccgaaaacgttgagaaatgcctaacacaagctttcgatcctctaggaatctcggattggaatagcttgttctggattgcccatccaggtggtcctgccattcttgatgcggttgaggctaaattgaacctagacaagaagaagttggaagccacaagacatgtactgtcagaatatggaaatatgagttctgcctgtgtcttattcatactcgacgaaatgagaaagaagtccttaaagggcgaaagagctactaccggcgaaggactagattggggagttttgtttggtttcggtcctggattgacaattgaaacagttgttttgcatagtattcccatggttaccaattaa

SEQ ID NO: 21 - DNA, 1179 bp, synthetic construct
  source 1..1179: mol_type = other DNA
atggctagcgtggaggaatttaggaatgcacagagagcgaaagggcctgctaccattttagcaatcggtactgcgactccagatcattgtgtataccaaagtgattatgcagactattatttcaaggtcaccaagtctgaacacatgaccgcattaaagaagaagtttaatagaatatgcgataagagcatgatcaagaaacgttatattcacttgacggaagaaatgttggaagaacatcctaatataggtgcttacatggcaccctctttgaatatcagacaggaaataattacggcagaagttcccaaattgggaaaagaggctgccttgaaggctttaaaagaatggggtcagcccaaatctaaaattacccacttagtattttgtacgacatcaggcgtcgaaatgccaggtgcggattacaaattagccaatttgttaggtttggaaccgtcagttagacgtgttatgttgtaccatcaaggatgctatgccggtgggacggttctgagaacagcgaaagatctagctgagaataacgcaggcgcaagagtattggtagtctgttccgaaataactgttgtcactttcagaggcccaagtgaggacgcgttggactcattagttggtcaggcactgtttggcgatggttctgccgctgtaattgtcggtagcgaccctgatataagtattgaaagacccctgttccaattggtttcagcagcacaaacttttattcctaatagtgctggtgctatcgctggtaatttaagagaagttggcttaacatttcatttgtggcctaatgttccaaccctgataagcgaaaacgtagagaaatgtcttacccaagcgttcgacccattaggaattagtgattggaactctcttttctggatcgcacacccaggaggcccagctatattagacgcagttgaagctaagttaaatttagataagaagaaattggaggcaacaagacatgtgttatccgagtacggaaatatgtcatcagcatgtgtgttgtttatattggacgagatgagaaagaagagtcttaagggagagagagctaccacaggagagggattggattggggtgtcttatttggttttggtccaggtctaacaattgaaacagtagtgttacactctattccaatggtcacaaattaa

SEQ ID NO: 22 - DNA, 1179 bp, synthetic construct
  source 1..1179: mol_type = other DNA
atggcatccgtggaagaatttagaaacgcacagagggcaaaaggtccagcaaccatactagctatcggcacagctacccctgatcattgcgtctatcagtcggactacgctgattattattttaaggttaccaaatcagaacacatgaccgcattgaagaagaagtttaacagaatatgtgacaaatcaatgattaagaagcgctatattcatctaactgaggagatgctggaggaacatccaaatattggtgcgtacatggcaccatccctaaacattcgccaagagattattacggctgaagttcccaagttaggcaaggaagcagctctgaaggcattaaaggagtggggccagcctaagagcaaaatcactcatcttgtattttgtacgacctctggtgtggaaatgcctggagctgactataaattagcgaacttgttgggcctagagccaagtgttagaagggtgatgctgtatcatcagggttgttatgcaggtggtactgtcttgaggacagccaaggatctggctgaaaataatgctggcgccagagtactcgtagtatgcagtgagatcaccgtcgtcacatttaggggaccatctgaagatgctttggattctctcgttggccaggctttattcggcgatggttccgctgctgtgatagtcggctcggatcctgacatatccatcgaacgccccttgtttcaattagttagcgcagcgcagacctttatacctaactcggccggggcaatagcaggtaatttgcgtgaagtcggattgacttttcatttgtggcctaacgtccccacgttgatttcagaaaatgtcgaaaagtgtttaacgcaagcattcgatcctctaggtatatctgattggaatagcctcttctggattgcacatcctggcgggcctgctattctggacgcggtcgaggctaagttaaatttggataagaagaagctggaagccaccagacatgtcctgtctgagtacgggaatatgtcaagtgcatgtgtgctctttatactggacgagatgaggaagaaatcgttaaagggtgagagagctactacgggtgaaggattagattggggcgtattattcggcttcggtccggggctcactatcgaaacagtagtcctgcatagtatccccatggtcaccaattga

SEQ ID NO: 23 - DNA, 1179 bp, synthetic construct
  source 1..1179: mol_type = other DNA
atggcctcagtagaagagtttcgtaatgctcaaagagccaagggcccagctacaattttagctataggcaccgctacgccagatcattgtgtttaccaatccgattacgcagattactatttcaaggtcacaaagagcgaacacatgactgccttaaagaagaaatttaaccgtatctgtgacaaatctatgatcaagaagcgttacatacatttgactgaagagatgttagaggagcaccctaacattggtgcctacatggcaccgtcgttaaatatccgtcaagaaattattacagctgaggtcccaaagttaggtaaggaagctgctcttaaagccttgaaggaatggggtcaacctaagagtaaaattacacatttggtcttttgtaccacttccggcgttgaaatgcctggcgccgattacaagttagctaacctattaggtctggaaccaagcgttcgtcgcgtaatgttataccatcagggatgttatgcaggtggtactgtattaaggaccgcaaaagacttggcagaaaataacgcgggcgccagagtattggtcgtgtgtagcgaaattacggttgtaacattcaggggtccatcagaggacgcactggacagtctcgtagggcaagcactatttggtgatggaagcgctgcggtcattgttggtagcgacccagacatatcaattgaaagacctcttttccaacttgtctctgctgcccaaacttttattccgaatagcgccggggctatcgcgggtaatcttagagaagtgggactgacgtttcatttatggccaaatgtgcccacacttataagcgaaaatgtcgaaaaatgtcttacgcaggcattcgatcctcttggtatatcggattggaactctctcttttggatcgcccatccaggtggtcctgcaattctggatgctgtagaagcaaaactaaacctggacaagaagaaactggaagctacaagacatgtcttgtcggaatacgggaacatgagttcggcatgtgtactttttattttagatgagatgcgtaaaaagtctctgaaaggtgagcgtgcaacaaccggtgaaggtttggactggggtgtcttgttcggattcggtcccggcttaaccatcgaaactgtagttctacattctattccaatggttactaattaa

SEQ ID NO: 24 - DNA, 1179 bp, synthetic construct
  source 1..1179: mol_type = other DNA
atggcttcagtcgaggagtttagaaatgctcagagggccaagggtcctgccacaatattagctataggtactgccaccccagatcactgtgtctatcaaagtgactatgctgactattattttaaagtcacaaaaagtgagcacatgactgcattgaaaaagaaattcaataggatatgtgataaatcaatgatcaaaaagagatacattcatctaactgaggaaatgttagaagagcatccaaatattggtgcatatatggctccatccttaaatatcagacaggaaataataaccgctgaggtgcctaaactgggtaaagaagctgcattaaaagcattaaaagaatggggtcagcctaaatcaaagattacgcatctagtattttgcacaacgtctggtgtcgaaatgcctggagccgattacaaactagcaaatttactaggtcttgaaccttctgtccgtcgagtaatgttataccaccaaggttgctacgcaggcggaaccgttctaaggactgccaaggacttggcagaaaataacgctggtgcaagggttttagtggtttgttctgaaatcactgtagtcacatttaggggtccctctgaagatgcattagactctttagttgggcaagcactgttcggggatgggtctgcggccgttatagtaggttcagatcctgacatttctatcgaaaggcctctgtttcaactggtatctgctgcccaaacttttattcctaacagcgctggtgcaatcgccgggaacctccgagaagtaggtcttacatttcatctatggcctaatgtccctactttgatttccgagaatgtagagaaatgcctgactcaggcctttgatcctttgggcatatctgattggaactcactattttggattgcacaccccggaggtcccgcaattttggatgccgtggaggctaaattaaatttagataagaagaaactcgaagcaactagacatgtattatcagagtacggcaatatgtctagtgcttgtgttttatttattttagacgaaatgcgtaaaaagtctttaaagggagagagggctactacaggagaaggattagattggggtgttttgtttggtttcggacccggtttaacgatcgaaacagttgttctgcatagtatccctatggtgaccaattga

SEQ ID NO: 25 - DNA, 1179 bp, synthetic construct
  source 1..1179: mol_type = other DNA
atggcatcggtagaagagttcagaaatgcacagagggctaaaggccctgccacaatcctagcaattggtactgcaactcccgatcattgcgtttatcaaagtgattatgccgactattattttaaagttacgaaatcagaacacatgactgctcttaaaaagaaattcaacagaatatgtgacaagagtatgattaaaaagagatacattcacttgacagaagagatgttggaggagcatcctaatatcggcgcttacatggcaccttcattgaacattcgtcaagaaataattactgccgaggttcctaaactcggcaaagaagcagcacttaaggcacttaaggaatggggtcagccaaagtcaaagatcacacatttggtcttttgtacaacctctggagttgagatgccaggcgctgattataaattggctaatcttttaggattagagccaagtgttaggcgggtgatgctatatcaccaaggttgttatgcaggtggtactgttttgaggacagccaaggatctggccgaaaataatgctggggccagagtcctggttgtttgctccgagataactgttgttacatttcgcgggccttcagaagatgcactggattctcttgtgggacaggcgctgtttggtgatgggtccgctgccgtgatcgtaggctctgatccagatatatcaattgagaggcctttatttcagttggtgtctgccgctcagacattcatccctaattccgcgggagcgatagctggtaatctaagagaggttggcttgacatttcacttatggcctaatgtgccaacattgatctctgagaacgtcgaaaagtgcctaacccaagcatttgacccattaggaattagcgactggaatagtttattttggatagcacaccctggaggtccggctatattggatgctgtggaagcaaagctaaatctggataagaagaagctagaagcaacaagacacgtactatctgaatacggaaatatgagcagtgcttgtgttctatttattcttgatgagatgcgtaaaaagagtttaaatggagaaagagccaccacaggtgaagggctagactggggcgttttatttggcttcggtccaggtctgacaatcgaaacggtcgtcttacactcaattccaatggttacaaattga

SEQ ID NO: 26 - protein, 563 aa, Saccharomyces cerevisiae
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 27 - DNA, 1692 bp, Saccharomyces cerevisiae
  source 1..1692: mol_type = genomic DNA
atgtctgaaataaccttaggtaaatatttatttgaaagattgagccaagtcaactgtaacaccgtcttcggtttgccaggtgactttaacttgtctcttttggataagctttatgaagtcaaaggtatgagatgggctggtaacgctaacgaattgaacgctgcctatgctgctgatggttacgctcgtatcaagggtatgtcctgtattattaccaccttcggtgttggtgaattgtctgctttgaatggtattgccggttcttacgctgaacatgtcggtgttttgcacgttgttggtgttccatccatctcttctcaagctaagcaattgttgttgcatcataccttgggtaacggtgacttcactgttttccacagaatgtctgccaacatttctgaaaccactgccatgatcactgatattgctaacgctccagctgaaattgacagatgtatcagaaccacctacactacccaaagaccagtctacttgggtttgccagctaacttggttgacttgaacgtcccagccaagttattggaaactccaattgacttgtctttgaagccaaacgacgctgaagctgaagctgaagttgttagaactgttgttgaattgatcaaggatgctaagaacccagttatcttggctgatgcttgtgcttctagacatgatgtcaaggctgaaactaagaagttgatggacttgactcaattcccagtttacgtcaccccaatgggtaagggtgctattgacgaacaacacccaagatacggtggtgtttacgttggtaccttgtctagaccagaagttaagaaggctgtagaatctgctgatttgatattgtctatcggtgctttgttgtctgatttcaataccggttctttctcttactcctacaagaccaaaaatatcgttgaattccactctgaccacatcaagatcagaaacgccaccttcccaggtgttcaaatgaaatttgccttgcaaaaattgttggatgctattccagaagtcgtcaaggactacaaacctgttgctgtcccagctagagttccaattaccaagtctactccagctaacactccaatgaagcaagaatggatgtggaaccatttgggtaacttcttgagagaaggtgatattgttattgctgaaaccggtacttccgccttcggtattaaccaaactactttcccaacagatgtatacgctatcgtccaagtcttgtggggttccattggtttcacagtcggcgctctattgggtgctactatggccgctgaagaacttgatccaaagaagagagttattttattcattggtgacggttctctacaattgactgttcaagaaatctctaccatgattagatggggtttgaagccatacatttttgtcttgaataacaacggttacaccattgaaaaattgattcacggtcctcatgccgaatataatgaaattcaaggttgggaccacttggccttattgccaacttttggtgctagaaactacgaaacccacagagttgctaccactggtgaatgggaaaagttgactcaagacaaggacttccaagacaactctaagattagaatgattgaagttatgttgccagtctttgatgctccacaaaacttggttaaacaagctcaattgactgccgctactaacgctaaacaataa

SEQ ID NO: 28 - DNA, 667 bp, synthetic construct
  source 1..667: mol_type = other DNA
tcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 29 - protein, 2233 aa, synthetic construct
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLADGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVAVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 30 - DNA, 6702 bp, synthetic construct
  source 1..6702: mol_type = other DNA
atgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataatggtattgccgccgtgaaagaaattagatccgtcagaaaatgggcatacgagacgttcggcgatgacagaaccgtccaattcgtcgccatggccaccccagaagatctggaggccaacgcagaatatatccgtatggccgatcaatacattgaagtgccaggtggtactaataataacaactacgctaacgtagacttgatcgtagacatcgccgaaagagcagacgtagacgccgtatgggctggctggggtcacgcctccgagaatccactattgcctgaaaaattgtcccagtctaagaggaaagtcatctttattgggcctccaggtaacgccatgaggtctttaggtgataaaatctcctctaccattgtcgctcaaagtgctaaagtcccatgtattccatggtctggtaccggtgttgacaccgttcacgtggacgagaaaaccggtctggtctctgtcgacgatgacatctatcaaaagggttgttgtacctctcctgaagatggtttacaaaaggccaagcgtattggttttcctgtcatgattaaggcatccgaaggtggtggtggtaaaggtatcagacaagttgaacgtgaagaagatttcatcgctttataccaccaggcagccaacgaaattccaggctcccccattttcatcatgaagttggccggtagagcgcgtcacttggaagttcaactgctagcagatcagtacggtacaaatatttccttgttcggtagagactgttccgttcagagacgtcatcaaaaaattatcgaagaagcaccagttacaattgccaaggctgaaacatttcacgagatggaaaaggctgccgtcagactggggaaactagtcggttatgtctctgccggtaccgtggagtatctatattctcatgatgatggaaaattctactttttagaattgaacccaagattacaagtcgagcatccaacaacggaaatggtctccggtgttaacttacctgcagctcaattacaaatcgctatgggtatccctatgcatagaataagtgacattagaactttatatggtatgaatcctcattctgcctcagaaatcgatttcgaattcaaaactcaagatgccaccaagaaacaaagaagacctattccaaagggtcattgtaccgcttgtcgtatcacatcagaagatccaaacgatggattcaagccatcgggtggtactttgcatgaactaaacttccgttcttcctctaatgtttggggttacttctccgtgggtaacaatggtaatattcactccttttcggactctcagttcggccatatttttgcttttggtgaaaatagacaagcttccaggaaacacatggttgttgccctgaaggaattgtccattaggggtgatttcagaactactgtggaatacttgatcaaacttttggaaactgaagatttcgaggataacactattaccaccggttggttggacgatttgattactcataaaatgaccgctgaaaagcctgatccaactcttgccgtcatttgcggtgccgctacaaaggctttcttagcatctgaagaagcccgccacaagtatatcgaatccttacaaaagggacaagttctatctaaagacctactgcaaactatgttccctgtagattttatccatgagggtaaaagatacaagttcaccgtagctaaatccggtaatgaccgttacacattatttatcaatggttctaaatgtgatatcatactgcgtcaactagctgatggtggtcttttgattgccataggcggtaaatcgcataccatctattggaaagaagaagttgctgctacaagattatccgttgactctatgactactttgttggaagttgaaaacgatccaacccagttgcgtactccatcccctggtaaattggttaaattcttggtggaaaatggtgaacacattatcaagggccaaccatatgcagaaattgaagttatgaaaatgcaaatgcctttggtttctcaagaaaatggtatcgtccagttattaaagcaacctggttctaccattgttgcaggtgatatcatggctattatgactcttgacgatccatccaaggtcaagcacgctctaccatttgaaggtatgctgccagattttggttctccagttatcgaaggaaccaaacctgcctataaattcaagtcattagtgtctactttggaaaacattttgaagggttatgacaaccaagttattatgaacgcttccttgcaacaattgatagaggttttgagaaatccaaaactgccttactcagaatggaaactacacatctctgctttacattcaagattgcctgctaagctagatgaacaaatggaagagttagttgcacgttctttgagacgtggtgctgttttcccagctagacaattaagtaaattgattgatatggccgtgaagaatcctgaatacaaccccgacaaattgctgggcgccgtcgtggaaccattggcggatattgctcataagtactctaacgggttagaagcccatgaacattctatatttgtccatttcttggaagaatattacgaagttgaaaagttattcaatggtccaaatgttcgtgaggaaaatatcattctgaaattgcgtgatgaaaaccctaaagatctagataaagttgcgctaactgttttgtctcattcgaaagtttcagcgaagaataacctgatcctagctatcttgaaacattatcaaccattgtgcaagttatcttctaaagtttctgccattttctctactcctctacaacatattgttgaactagaatctaaggctaccgctaaggtcgctctacaagcaagagaaattttgattcaaggcgctttaccttcggtcaaggaaagaactgaacaaattgaacatatcttaaaatcctctgttgtgaaggttgcctatggctcatccaatccaaagcgctctgaaccagatttgaatatcttgaaggacttgatcgattctaattacgttgtgttcgatgttttacttcaattcctaacccatcaagacccagttgtgactgctgcagctgctcaagtctatattcgtcgtgcttatcgtgcttacaccataggagatattagagttcacgaaggtgtcacagttccaattgttgaatggaaattccaactaccttcagctgcgttctccacctttccaactgttaaatctaaaatgggtatgaacagggctgttgctgtttcagatttgtcatatgttgcaaacagtcagtcatctccgttaagagaaggtattttgatggctgtggatcatttagatgatgttgatgaaattttgtcacaaagtttggaagttattcctcgtcaccaatcttcttctaacggacctgctcctgatcgttctggtagctccgcatcgttgagtaatgttgctaatgtttgtgttgcttctacagaaggtttcgaatctgaagaggaaattttggtaaggttgagagaaattttggatttgaataagcaggaattaatcaatgcttctatccgtcgtatcacatttatgttcggttttaaagatgggtcttatccaaagtattatacttttaacggtccaaattataacgaaaatgaaacaattcgtcacattgagccggctttggccttccaactggaattaggaagattgtccaacttcaacattaaaccaattttcactgataatagaaacatccatgtctacgaagctgttagtaagacttctccattggataagagattctttacaagaggtattattagaacgggtcatatccgtgatgacatttctattcaagaatatctgacttctgaagctaacagattgatgagtgatatattggataatttagaagtcaccgacacttcaaattctgatttgaatcatatcttcatcaacttcattgcggtgtttgatatctctccagaagatgtcgaagccgccttcggtggtttcttagaaagatttggtaagagattgttgagattgcgtgtttcttctgccgaaattagaatcatcatcaaagatcctcaaacaggtgccccagtaccattgcgtgccttgatcaataacgtttctggttatgttatcaaaacagaaatgtacaccgaagtcaagaacgcaaaaggtgaatgggtatttaagtctttgggtaaacctggatccatgcatttaagacctattgctactccttaccctgttaaggaatggttgcaaccaaaacgttataaggcacacttgatgggtaccacatatgtctatgacttcccagaattattccgccaagcatcgtcatcccaatggaaaaatttctctgcagatgttaagttaacagatgatttctttatttccaacgagttgattgaagatgaaaacggcgaattaactgaggtggaaagagaacctggtgccaacgctattggtatggttgcctttaagattactgtaaagactcctgaatatccaagaggccgtcaatttgttgttgttgctaacgatatcacattcaagatcggttcctttggtccacaagaagacgaattcttcaataaggttactgaatatgctagaaagcgtggtatcccaagaatttacttggctgcaaactcaggtgccagaattggtatggctgaagagattgttccactatttcaagttgcatggaatgatgctgccaatccggacaagggcttccaatacttatacttaacaagtgaaggtatggaaactttaaagaaatttgacaaagaaaattctgttctcactgaacgtactgttataaacggtgaagaaagatttgtcatcaagacaattattggttctgaagatgggttaggtgtcgaatgtctacgtggatctggtttaattgctggtgcaacgtcaagggcttaccacgatatcttcactatcaccttagtcacttgtagatccgtcggtatcggtgcttatttggttcgtttgggtcaaagagctattcaggtcgaaggccagccaattattttaactggtgctcctgcaatcaacaaaatgctgggtagagaagtttatacttctaacttacaattgggtggtactcaaatcatgtataacaacggtgtttcacatttgactgctgttgacgatttagctggtgtagagaagattgttgaatggatgtcttatgttccagccaagcgtaatatgccagttcctatcttggaaactaaagacacatgggatagaccagttgatttcactccaactaatgatgaaacttacgatgtaagatggatgattgaaggtcgtgagactgaaagtggatttgaatatggtttgtttgataaagggtctttctttgaaactttgtcaggatgggccaaaggtgttgtcgttggtagagcccgtcttggtggtattccactgggtgttattggtgttgaaacaagaactgtcgagaacttgattcctgctgatccagctaatccaaatagtgctgaaacattaattcaagaacctggtcaagtttggcatccaaactccgccttcaagactgctcaagctatcaatgactttaacaacggtgaacaattgccaatgatgattttggccaactggagaggtttctctggtggtcaacgtgatatgttcaacgaagtcttgaagtatggttcgtttattgttgacgcattggtggattacaaacaaccaattattatctatatcccacctaccggtgaactaagaggtggttcatgggttgttgtcgatccaactatcaacgctgaccaaatggaaatgtatgccgacgtcaacgctagagctggtgttttggaaccacaaggtatggttggtatcaagttccgtagagaaaaattgctggacaccatgaacagattggatgacaagtacagagaattgagatctcaattatccaacaagagtttggctccagaagtacatcagcaaatatccaagcaattagctgatcgtgagagagaactattgccaatttacggacaaatcagtcttcaatttgctgatttgcacgataggtcttcacgtatggtggccaagggtgttatttctaaggaactggaatggaccgaggcacgtcgtttcttcttctggagattgagaagaagattgaacgaagaatatttgattaaaaggttgagccatcaggtaggcgaagcatcaagattagaaaagatcgcaagaattagatcgtggtaccctgcttcagtggaccatgaagatgataggcaagtcgcaacatggattgaagaaaactacaaaactttggacgataaactaaagggtttgaaattagagtcattcgctcaagacttagctaaaaagatcagaagcgaccatgacaatgctattgatggattatctgaagttatcaagatgttatctaccgatgataaagaaaaattgttgaagactttgaaataa

(19) United States
(12) Patent Application Publication
(10) Pub. No.: US 2026/0193680 A1
(43) Pub. Date: Jul. 9, 2026

(54) BIOSYNTHESIS OF DIHYDROCHALCONES AND DIHYDROSTILBENOIDS

(71) Applicant: Conagen Inc., Bedford, MA (US)

(72) Inventor: Grace Park, Acton, MA (US)

(21) Appl. No.: 19/132,792

(22) PCT Filed: Nov. 22, 2023
(86) PCT No.: PCT/US2023/080907

§ 371 Date: May 23, 2025

Related U.S. Application Data
(60) Provisional application No. 63/384,631, filed on Nov. 22, 2022.

(51) Int. Cl. C12P 7/22; C07C 45/78; C12N 9/02; C12P 7/26

(52) U.S. Cl. CPC C12P 7/22; C07C 45/78; C12N 9/001; C12P 7/26

(57) ABSTRACT

Provided herein is a method of increasing production of a dihydrodiphenylpropanoid derivative compound (e.g., dihydrostilbenoid or dihydrochalcone compound) in a recombinant host cell. The method comprising: reducing or eliminating expression of a gene encoding a double-bond reductase polypeptide or activity of the double-bond reductase polypeptide encoded thereby; expressing a recombinant gene encoding a double-bond reductase selected from the group consisting of a first polypeptide, second polypeptide, third polypeptide, and combinations thereof. The first polypeptide comprises an amino acid sequence having at least 90% identity to the amino acid sequence as set forth in SEQ ID NO: 3, the second polypeptide comprises an amino acid sequence having at least 90% identity to the amino acid sequence as set forth in SEQ ID NO: 5, and the third polypeptide comprises an amino acid sequence having at least 90% identity to the amino acid sequence as set forth in SEQ ID NO: 7.

### RELATED APPLICATION

This application claims the benefit under 35 U.S.C. § 119(e) to U.S. Provisional Application No. 63/384,631, filed on Nov. 22, 2022, the entire contents of which are incorporated herein by reference.

### REFERENCE TO AN ELECTRONIC SEQUENCE LISTING

The contents of the electronic sequence listing (C149770094WO00-SEQ-VLJ.xml; Size: 62,596 bytes; and Date of Creation: Nov. 22, 2023) is herein incorporated by reference in its entirety.

### BACKGROUND OF THE DISCLOSURE

Dihydroresveratrol is a side product from the resveratrol biosynthetic pathway (FIG. 1). It is speculated that a double-bond reductase or an enoyl reductase reduces the double bond in coumaroyl-CoA to generate dihydrocoumaroyl-CoA which is subsequently converted to dihydroresveratrol by stilbene synthase or phloretin by chalcone synthase (Eichenberger et al. 2017 Metabolic engineering 39. P. 80). In Saccharomyces cerevisiae, TSC13 was identified as a major double-bond reductase showing high activity on coumaroyl-CoA (Lehka et al. 2017. FEMS Yeast Research, fox004). TSC13 is an enzyme of the very long chain fatty acid synthase family. TSC 13 increased the production of phloretic acid and dihydroresveratrol in resveratrol-producing strains and also increased phloretin in naringenin-producing strains when overexpressed (Naesby et al., U.S. Pat. No. 10,760,062). DFG10 is another native double-bond reductase in Saccharomyces cerevisiae which exhibits high activity in the production of greater relative amounts of amounts phloretic acid but is less active for phloretin production than TSC13. It has also been reported that replacement of endogenous Saccharomyces cerevisiae TSC13 with enzyme MdECR (Malus domestica enoyl-ACP reductase) abolished phloretic acid production, yet failed to increase phloretin production (Lehka et al 2017. FEMS Yeast Research, fox004). TSC13 from Kluyveromyces lactis showed dramatic changes in the ratio of resveratrol and dihydroresveratol production (Naesby et al., U.S. Pat. No. 10,760,062), but also led to an overall decrease in stilbene production.

### SUMMARY OF THE DISCLOSURE

The present disclosure relates to the synthesis of dihydrochalcones and dihydrostilbenoids. More particularly, the present disclosure relates to recombinant yeast cells and biosynthetic methods for producing the dihydrochalcones and/or dihydrostilbenoids.

In a first aspect, disclosed herein is a method of increasing production of a dihydrodiphenylpropanoid derivative compound in a recombinant host cell. The method comprises: reducing or eliminating expression of a gene encoding a double-bond reductase polypeptide or activity of the double-bond reductase polypeptide encoded thereby; and expressing a recombinant gene encoding a double-bond reductase selected from the group consisting of a first polypeptide, second polypeptide, third polypeptide, and combinations thereof, wherein the first polypeptide comprises an amino acid sequence having at least 90% identity to the amino acid sequence as set forth in SEQ ID NO: 3, the second polypeptide comprises an amino acid sequence having at least 90% identity to the amino acid sequence as set forth in SEQ ID NO: 5, and the third polypeptide comprises an amino acid sequence having at least 90% identity to the amino acid sequence as set forth in SEQ ID NO: 7, wherein the product dihydrophenylpropanoid derivative compound is a dihydrostilbenoid or a dihydrochalcone compound. In a first example, the first polypeptide comprises an amino acid sequence having at least 95%, at least 99%, or 100% identity to the amino acid sequence as set forth in SEQ ID NO: 3. In a second example, the first polypeptide comprises an amino acid sequence having at least 95%, at least 99%, or 100% identity to the amino acid sequence as set forth in SEQ ID NO: 5. In a third example, the first polypeptide comprises an amino acid sequence having at least 95%, at least 99%, or 100% identity to the amino acid sequence as set forth in SEQ ID NO: 7. The gene whose expression is reduced or eliminated may encode an enoyl reductase, for instance a S. cerevisiae trans-2-enoyl-CoA reductase TSC13. In a representative embodiment, expression of the enoyl reductase gene is eliminated by deletion of the gene. The dihydrostilbenoid may be dihydroresveratrol. The dihydrochalcone may be phloretin. The recombinant host cell may be a yeast cell, for example a cell from the Saccharomyces cerevisiae species.

In a second aspect, the present disclosure provides recombinant host cell capable of producing a dihydrodiphenylpropanoid compound. The recombinant cell comprises a gene encoding a double-bond reductase polypeptide, wherein expression of the gene or activity of the double-bond reductase polypeptide encoded thereby is reduced or eliminated; and a recombinant gene encoding a double-bond reductase selected from the group consisting of a first polypeptide, second polypeptide, third polypeptide, and combinations thereof, wherein the first polypeptide comprises an amino acid sequence having at least 90% identity to the amino acid sequence as set forth in SEQ ID NO: 3, the second polypeptide comprises an amino acid sequence having at least 90% identity to the amino acid sequence as set forth in SEQ ID NO: 5, and the third polypeptide comprises an amino acid sequence having at least 90% identity to the amino acid sequence as set forth in SEQ ID NO: 7. In a first example, the first polypeptide comprises an amino acid sequence having at least 95%, at least 99%, or 100% identity to the amino acid sequence as set forth in SEQ ID NO: 3. In a second example, the first polypeptide comprises an amino acid sequence having at least 95%, at least 99%, or 100% identity to the amino acid sequence as set forth in SEQ ID NO: 5. In a third example, the first polypeptide comprises an amino acid sequence having at least 95%, at least 99%, or 100% identity to the amino acid sequence as set forth in SEQ ID NO: 7. The gene whose expression is reduced or eliminated encodes may be an enoyl reductase, for example a S. cerevisiae trans-2-enoyl-CoA reductase TSC13. In a representative embodiment, expression of the enoyl reductase gene is eliminated by deletion of the gene. The dihydrostilbenoid may be dihydroresveratrol. The dihydrochalcone may be phloretin. The recombinant host cell may be a yeast cell. Typical yeast cells include Saccharomycetes, for examples a from the Saccharomyces cerevisiae species.

In a third aspect, there is provided a biosynthetic method whereby a recombinant cell according to the above second aspect is be cultivated in a culture medium under conditions in which the recombinant genes are expressed, and wherein the product compound is synthesized by the recombinant host cell. In a representative embodiment, the product compound is dihydrochalcone compound such as phloretin. In a further embodiment, the product compound is a dihydrostilbenoid compound, for example dihydrostilbene.

In a fourth aspect, there is provided a biosynthetic method of producing a compound of formula (V):

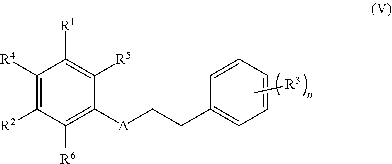

or a pharmaceutically acceptable salt thereof, wherein
A is a bond or C═O;
n is an integer 0, 1, 2, 3, or 4;
R¹ is hydrogen or —OR¹¹;
wherein each R¹¹ is independently hydrogen, C₁-C₆ alkyl, or glycosyl;
R² is hydrogen, —OR¹¹, C₁-C₁₂ alkyl, or C₂-C₁₂ alkenyl, wherein alkyl and alkenyl are optionally substituted with one or more R⁷;
   or R² and R⁶ together with the atoms to which they are attached form a 5- to 7-member heterocyclyl optionally substituted with one or more R⁷ groups;
   or R² and R⁴ together with the atoms to which they are attached form a 5- to 7-member heterocyclyl optionally substituted with one or more R⁷ groups;
R³ is independently selected from nitro, C₁-C₆ alkyl, C₂-C₆ alkenyl, C₁-C₆ haloalkyl, C₁-C₆ hydroxyalkyl, —OR¹², —N(R¹²)2, —C(O)R¹², —C(O)OR¹², —C(O)N(R¹²)₂, and —S(O)₂R¹², wherein each R¹² is independently hydrogen or C₁-C₆ alkyl;
R⁴ is hydrogen, —OR¹¹, C₁-C₁₂ alkyl, or C₂-C₁₂ alkenyl, wherein alkyl and alkenyl are optionally substituted with one or more R⁷;
   or R⁴ and R² together with the atoms to which they are attached form a 5- to 7-member heterocyclyl optionally substituted with one or more R⁷ groups;
R⁵ is hydrogen or —OR¹¹; and
R⁶ is hydrogen, C₁-C₆ alkyl, C₂-C₆ alkenyl, —OR¹¹, or —N(R¹⁰)₂, wherein each R¹⁰ is independently hydrogen or C₁-C₆ alkyl, and wherein alkyl and alkenyl are optionally substituted with one or more R⁸; or R⁶ and R² together with the atoms to which they are attached form a 5- to 7-member heterocyclyl optionally substituted with one or more R⁸ groups;
   each R⁷ and R⁸ is independently halogen, cyano, nitro, C₁-C₆ alkyl, C₂-C₆ alkenyl, C₁-C₆ haloalkyl, C₁-C₆ hydroxyalkyl, —OR¹³, —SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl.

The method of this fourth aspect comprises growing a recombinant host cell according to the above second aspect in a culture medium under conditions in which the recombinant genes are expressed, and wherein the compound of formula (V) is synthesized by the recombinant host cell.

In a representative embodiment, the compound of formula (V) is a dihydrostilbenoid of formula (V-A):

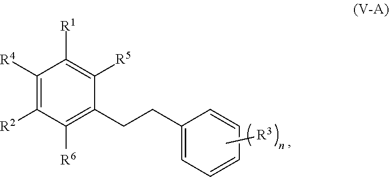

or a pharmaceutically acceptable salt thereof, wherein
n is an integer 0, 1, 2, 3, or 4;
R¹ is hydrogen or —OR¹¹;
wherein each R¹¹ is independently hydrogen, C₁-C₆ alkyl, or glycosyl;
R² is hydrogen, —OR¹¹, C₁-C₁₂ alkyl, or C₂-C₁₂ alkenyl, wherein alkyl and alkenyl are optionally substituted with one or more R⁷;
   or R² and R⁶ together with the atoms to which they are attached form a 5- to 7-member heterocyclyl optionally substituted with one or more R⁷ groups;
   or R² and R⁴ together with the atoms to which they are attached form a 5- to 7-member heterocyclyl optionally substituted with one or more R⁷ groups;
R³ is independently selected from nitro, C₁-C₆ alkyl, C₂-C₆ alkenyl, C₁-C₆ haloalkyl, C₁-C₆ hydroxyalkyl, —OR¹², —N(R¹²)2, —C(O)R¹², —C(O)OR¹², —C(O)N(R¹²)2, and —S(O)₂R¹², wherein each R¹² is independently hydrogen or C₁-C₆ alkyl;
R⁴ is hydrogen, —OR¹¹, C₁-C₁₂ alkyl, or C₂-C₁₂ alkenyl, wherein alkyl and alkenyl are optionally substituted with one or more R⁷;
   or R⁴ and R² together with the atoms to which they are attached form a 5- to 7-member heterocyclyl optionally substituted with one or more R⁷ groups;
R⁵ is hydrogen or —OR¹¹; and
R⁶ is hydrogen, C₁-C₆ alkyl, C₂-C₆ alkenyl, —OR¹¹, or —N(R¹⁰)₂, wherein each R¹⁰ is independently hydrogen or C₁-C₆ alkyl, and wherein alkyl and alkenyl are optionally substituted with one or more R⁸; or R⁶ and R² together with the atoms to which they are attached form a 5- to 7-member heterocyclyl optionally substituted with one or more R⁸ groups;
each R⁷ and R⁸ is independently halogen, cyano, nitro, C₁-C₆ alkyl, C₂-C₆ alkenyl, C₁-C₆haloalkyl, C₁-C₆ hydroxyalkyl, —OR¹³, —SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl.

In another representative embodiment, the compound of formula (V) is a dihydrochalcone compound of formula (V-B):

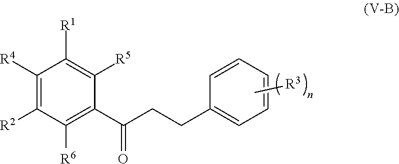

or a pharmaceutically acceptable salt thereof, wherein
n is an integer 0, 1, 2, 3, or 4;
R¹ is hydrogen or —OR¹¹;
wherein each R¹¹ is independently hydrogen, C₁-C₆ alkyl, or glycosyl;
R² is hydrogen, —OR¹¹, C₁-C₁₂ alkyl, or C₂-C₁₂ alkenyl, wherein alkyl and alkenyl are optionally substituted with one or more R⁷;
   or R² and R⁶ together with the atoms to which they are attached form a 5- to 7-member heterocyclyl optionally substituted with one or more R⁷ groups;
   or R² and R⁴ together with the atoms to which they are attached form a 5- to 7-member heterocyclyl optionally substituted with one or more R⁷ groups;
R³ is independently selected from nitro, C₁-C₆ alkyl, C₂-C₆ alkenyl, C₁-C₆ haloalkyl, C₁-C₆ hydroxyalkyl, —OR¹², —N(R¹²)2, —C(O)R¹², —C(O)OR¹², —C(O)N(R¹²)2, and —S(O)₂R¹², wherein each R¹² is independently hydrogen or C₁-C₆ alkyl;
R⁴ is hydrogen, —OR¹¹, C₁-C₁₂ alkyl, or C₂-C₁₂ alkenyl, wherein alkyl and alkenyl are optionally substituted with one or more R⁷;
   or R⁴ and R² together with the atoms to which they are attached form a 5- to 7-member heterocyclyl optionally substituted with one or more R⁷ groups;
R⁵ is hydrogen or —OR¹¹; and
R⁶ is hydrogen, C₁-C₆ alkyl, C₂-C₆ alkenyl, —OR¹¹, or —N(R¹⁰)₂, wherein each R¹⁰ is independently hydrogen or C₁-C₆ alkyl, and wherein alkyl and alkenyl are optionally substituted with one or more R⁸; or R⁶ and R² together with the atoms to which they are attached form a 5- to 7-member heterocyclyl optionally substituted with one or more R⁸ groups;
each R⁷ and R⁸ is independently halogen, cyano, nitro, C₁-C₆ alkyl, C₂-C₆ alkenyl, C₁-C₆haloalkyl, C₁-C₆ hydroxyalkyl, —OR¹³, —SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 illustrates a biosynthesis of resveratrol and dihydroresveratrol from p-coumaric acid.

FIG. 2 illustrates a of naringenin and phloretin from p-coumaric acid.

FIG. 3 includes an alignment of a number of TSC13 polypeptides (from top to bottom, SEQ ID NOs: 7, 5, 3, 1, and 9).

FIG. 4 illustrates resveratrol and dihydroresveratrol production yields from strains expressing a number of TSC13 homologs.

FIG. 5 illustrates the share of dihydroresveratrol out of overall stilbenoid production in a number of strains.

FIG. 6 illustrates dihydroresveratrol and resveratrol production from the strain expressing Saccharomyces paradoxus TSC13.

FIG. 7 illustrates percentages of dihydroresveratrol out of total stilbene production in strains expressing Saccharomyces paradoxus TSC13.

FIG. 8 illustrates dihydroresveratrol and resveratrol production in a strain expressing Yarrowia lipolitica TSC13.

FIG. 9 illustrates phloretin and naringenin production in strains expressing a number of TSC13 homologs.

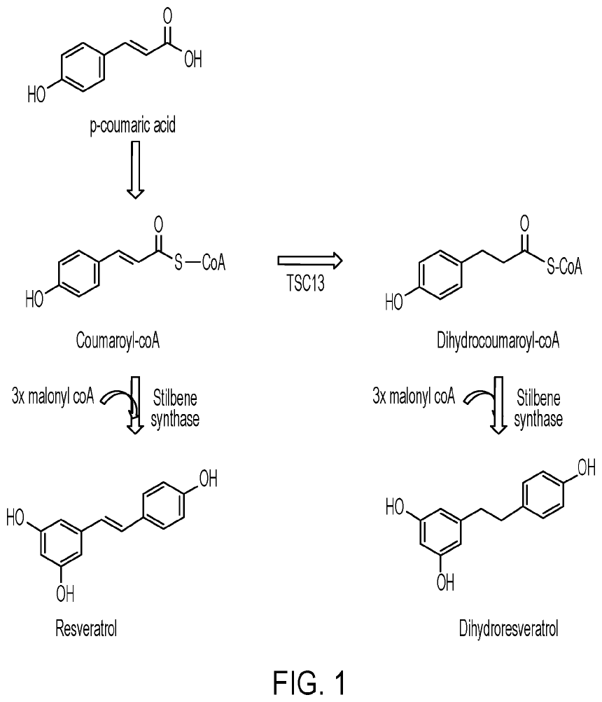

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Because many dihydrophenylpropanoid derivatives are useful as, inter alia, pharmaceutical compounds, there is a need for efficient methods of their production. For example, the dihydrochalcones phlorizin and phloretin are useful for controlling blood sugar levels, as well as other potential uses to improve human health.

Accordingly, provided herein are materials and methods useful for biosynthesis of dihydrophenylpropanoid derivatives, including dihydrochalcones and dihydrostilbenes. In one aspect, the present disclosure provides recombinant hosts and methods for biosynthesis of phloretin and other dihydrochalcones. In another aspect, the disclosure provides recombinant hosts and methods for biosynthesis of dihydroresveratrol and other dihydrostilbenes.

Before describing the disclosed methods and compositions in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of this invention.

For the purposes of describing and defining this invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Methods well known to those skilled in the art can be used to construct the genetic expression constructs and recombinant cells disclosed herein. These methods include in vitro recombinant DNA techniques, synthetic techniques, in vivo recombination techniques, and polymerase chain reaction (PCR) techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

As used herein, the terms “microorganism,” “microorganism host,” “microorganism host cell,” “recombinant host,” “host cell,” and “recombinant host cell” can be used interchangeably. As used herein, the term “recombinant host” is intended to refer to a host, the genome of which has been augmented by at least one DNA sequence. Such DNA sequences include but are not limited to genes or DNA sequences that are not naturally present, that are not normally transcribed into RNA, nor translated into protein (“expressed”) natively in the cell, and other genes or DNA sequences one desires to introduce into a host. It will be appreciated that typically the genome of a recombinant host described herein is augmented through stable introduction of one or more recombinant genes. Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

As used herein, the term “gene” refers to a polynucleotide unit comprised of at least one of the DNA sequences disclosed herein, or any DNA sequences encoding the amino acid sequences disclosed herein, or any DNA sequence that hybridizes to the complement of the coding sequence disclosed herein. Preferably, the term includes coding and non-coding regions, and preferably all sequences necessary for normal gene expression including promoters, enhancers, and other regulatory sequences.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species, or can be a DNA sequence that originated from or is present in the same species, but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. The recombinant genes are particularly encoded by cDNA.

A recombinant gene encoding a polypeptide described herein comprises the coding sequence for that polypeptide, operably linked in sense orientation to one or more regulatory regions suitable for expressing the polypeptide. Because many microorganisms can be capable of expressing multiple gene products from a polycistronic mRNA, multiple polypeptides are optionally expressed under the control of a single regulatory region for those microorganisms, if desired. A coding sequence and a regulatory region are operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene. In many cases, the coding sequence for a polypeptide described herein is identified in a species other than the recombinant microorganism, i.e., is a heterologous nucleic acid. Thus, the coding sequence can be from other prokaryotic or eukaryotic microorganisms, from plants or from animals. In some cases, however, the coding sequence is a sequence that is native to the microorganism and is being reintroduced into that organism.

As used herein, the term “engineered biosynthetic pathway” refers to a biosynthetic pathway that occurs in a recombinant host, as described herein, and does not naturally occur in the host. In some embodiments, the engineered biosynthetic pathway comprises enzymes naturally produced by the host, wherein in certain embodiments the extent and amount of expression of the genes encoding these enzymes are altered in the recombinant host; in some embodiments these enzymes are overexpressed in the recombinant host.

As used herein, the term “endogenous” gene refers to a gene that originates from and is produced or synthesized within a particular organism, tissue, or cell.

As used herein, the terms “heterologous sequence” and “heterologous coding sequence” are used to describe a sequence derived from a species other than the recombinant host. In some embodiments, the recombinant host is an S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned at further distance, for example as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements. One or more genes can be combined in a recombinant nucleic acid construct in “modules” useful for a discrete aspect of compound production. Combining a plurality of genes in a module, particularly a polycistronic module, facilitates the use of the module in a variety of species. In addition to genes useful for compound production, a recombinant construct typically also contains an origin of replication, and one or more selectable markers for maintenance of the construct in appropriate species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular microorganism is obtained, using appropriate codon bias tables for that microorganism. Nucleic acids may also be optimized to a GC-content preferable to a particular microorganism, and/or to reduce the number of repeat sequences. As isolated nucleic acids, these modified sequences can exist as purified molecules and can be incorporated into a vector or a virus for use in constructing modules for recombinant nucleic acid constructs. In addition, heterologous nucleic acids can be modified for increased or even optimal expression in the relevant microorganism. Thus, in some embodiments of the methods and compositions disclosed herein, heterologous nucleic acids have been codon optimized for expression in the relevant microorganism. Codon optimization may be performed by routine methods known in the art (See e.g., Welch, M., et al. (2011), Methods in Enzymology 498:43-66).

As used herein, the term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96%, at least 97%, at least 98%, at least 99%, and even 100% identical.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, MA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, WI). “Gap” utilizes the algorithm of Needleman and Wunsch (Needleman and Wunsch, JOURNAL OF MOLECULAR BIOLOGY 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981, Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

As used herein, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

As used herein, the terms “chalcone” and “chalconoid” are interchangeable and refer to derivatives the compound of formula (I):

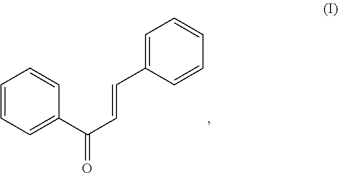 [SMILES: O=C(/C=C/c1ccccc1)c1ccccc1]

wherein formula (I) may be substituted at one or more suitable positions. Exemplary substituents include, but are not limited to, halogen, cyano, nitro, C₁-C₆ alkyl, C₁-C₆ haloalkyl, C₁-C₆ hydroxyalkyl, hydroxy, C₁-C₆ alkoxy, thiol, C₁-C₆ alkylthio, amino, C₁-C₆ alkyl amino, di-C₁-C₆ alkyl amino, carboxyl, C₁-C₆ alkoxycarbonyl, amido, and glycosyl.

As used herein, the terms “stilbene” and “stilbenoid” are interchangeable and refer to compounds based on the compound of formula (II):

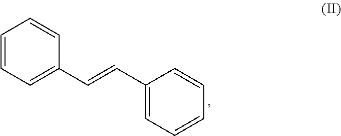 [SMILES: C(=C\c1ccccc1)/c1ccccc1]

wherein formula (II) may be substituted at one or more suitable positions. Exemplary substituents include, but are not limited to, halogen, cyano, nitro, C₁-C₆ alkyl, C₁-C₆ haloalkyl, C₁-C₆ hydroxyalkyl, hydroxy, C₁-C₆ alkoxy, thiol, C₁-C₆ alkylthio, amino, C₁-C₆ alkyl amino, di-C₁-C₆ alkyl amino, carboxyl, C₁-C₆ alkoxycarbonyl, amido, and glycosyl.

As used herein, the terms “dihydrochalcone” and “dihydrochalconoid” are interchangeable and refer to derivatives the compound of formula (III):

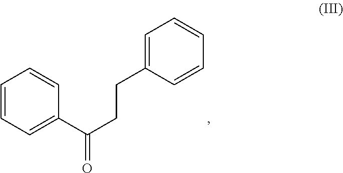 [SMILES: O=C(CCc1ccccc1)c1ccccc1]

wherein formula (III) may be substituted at one or more suitable positions. Exemplary substituents include, but are not limited to, halogen, cyano, nitro, C₁-C₆ alkyl, C₁-C₆ haloalkyl, C₁-C₆ hydroxyalkyl, hydroxy, C₁-C₆ alkoxy, thiol, C₁-C₆ alkylthio, amino, C₁-C₆ alkyl amino, di-C₁-C₆ alkyl amino, carboxyl, C₁-C₆ alkoxycarbonyl, amido, and glycosyl.

As used herein, the terms “dihydrostilbene” and “dihydrostilbenoid” are interchangeable and refer to compounds based on the compound of formula (IV):

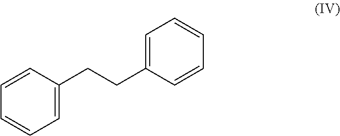 [SMILES: c1ccc(CCc2ccccc2)cc1]

wherein formula (IV) may be substituted at one or more suitable positions. Exemplary substituents include, but are not limited to, halogen, cyano, nitro, C₁-C₆ alkyl, C₁-C₆ haloalkyl, C₁-C₆ hydroxyalkyl, hydroxy, C₁-C₆ alkoxy, thiol, C₁-C₆ alkylthio, amino, C₁-C₆ alkyl amino, di-C₁-C₆ alkyl amino, carboxyl, C₁-C₆ alkoxycarbonyl, amido, and glycosyl.

As used herein, the term “phenylpropanoid” refers to compounds based on a 3-phenylprop-2-enoate backbone. Examples of such compounds include, but are not limited to, cinnamic acid, coumaric acid, caffeic acid, ferulic acid, 5-hydroxyferulic acid, sinapinic acid, cinnamoyl-CoA, p-coumaroyl-CoA, and the like.

As used herein, the terms “phenylpropanoid derivative” and “phenylpropanoid derivative compound” are interchangeable and refer to any compound derived from, synthesized from, or biosynthesized from a phenylpropanoid; i.e., a phenylpropanoid derivative includes any compound for which a phenylpropanoid compound is a precursor or intermediate. Examples of phenylpropanoid derivatives include, but are not limited to, stilbene compounds and chalcone compounds. Specific examples of phenylpropanoid derivatives include, but are not limited to, naringenin, resveratrol, pinosylvin, pinocembrin chalcone, and pinocembrin.

As used herein, the term “dihydrophenylpropanoid” refers to compounds based on a phenylpropanoate backbone. Examples of such compounds include, but are not limited to, dihydrocinnamic acid, phloretic acid, 3,4-dihydroxyhydrocinnamic acid, hydroferulic acid, dihydrocoumaroyl-CoA, dihydrocinnamoyl-CoA, and the like.

As used herein, the terms “dihydrophenylpropanoid derivative” and “dihydrophenylpropanoid derivative compound” are interchangeable and refer to any compound derived from, synthesized from, or biosynthesized from a dihydrophenylpropanoid; i.e. a dihydrophenylpropanoid derivative includes any compound for which a dihydrophenylpropanoid compound is a precursor or intermediate. Examples of dihydrophenylpropanoid derivatives include, but are not limited to, dihydrostilbenoid compounds and dihydrochalcone compounds. Specific examples of dihydrophenylpropanoid derivatives include, but are not limited to, phloretin, phlorizin, dihydropinosylvin, 3-O-methyldihydropinosylvin, 2-isoprenyl-3-O-methyldihydropinosylvin (amorfrutin 2; IUPAC: 3-methoxy-2-(3-methylbut-2-en-1-yl)-5-phenethylphenol), and dihydroresveratrol.

As used herein, the terms “phenylpropanoid pathway,” “phenylpropanoid derivative pathway,” “phenylpropanoid derivative synthesis pathway,” and “phenylpropanoid derivative biosynthesis pathway” are interchangeable and refer to any biosynthesis pathway in which a phenylpropanoid is a precursor or intermediate and in which a phenylpropanoid derivative compound is a product. Phenylpropanoid derivatives, such as chalcones and stilbenes, are biosynthesized according to phenylpropanoid derivative biosynthesis pathways.

As used herein, the terms “dihydrophenylpropanoid pathway,” “dihydrophenylpropanoid derivative pathway,” “dihydrophenylpropanoid derivative synthesis pathway,” and “dihydrophenylpropanoid derivative biosynthesis pathway” are interchangeable and refer to any biosynthesis pathway in which a phenylpropanoid or dihydrophenylpropanoid is a precursor or intermediate and in which a dihydrophenylpropanoid derivative compound is a product. Dihydrophenylpropanoid derivatives, such as dihydrochalcones and dihydrostilbenes, are biosynthesized according to dihydrophenylpropanoid derivative biosynthesis pathways.

As used herein, the term “alkyl” means a straight or branched chain hydrocarbon containing from 1 to 20 carbon atoms unless otherwise specified. The term “Cₘ-Cₙ alkyl” means an alkyl group having from m to n carbon atoms. For example, “C₁-C₆ alkyl” is an alkyl group having from one to six carbon atoms. Representative examples of alkyl include, but are not limited to, methyl, ethyl, n-propyl, isopropyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl” as used herein, means a straight or branched chain hydrocarbon containing from 2 to 20 carbons, unless otherwise specified, and containing at least one carbon-carbon double bond. The term “Cₘ-Cₙ alkenyl” means an alkenyl group having from m to n carbon atoms. For example, “C₂-C₆ alkenyl” is an alkenyl group having from one to six carbon atoms. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl, and 2-propyl-2-heptenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The terms “cyano” and “nitrile” as used herein, mean a —CN group.

The term “halogen” as used herein, means —Cl, —Br, —I or —F.

The term “haloalkyl” refers to an alkyl group, which is substituted with one or more halogen atoms.

The term “heterocyclyl” as used herein, means a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle may be attached through either cyclic moiety (e.g., either through heterocycle or through phenyl.) Representative examples of heterocycle include, but are not limited to, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, trithianyl, 2,3-dihydrobenzofuran-2-yl, and indolinyl.

The term “hydroxyalkyl” refers to an alkyl group, which is substituted with one or more —OH groups.

As used herein, the term “glycosyl” means is a univalent radical obtained by removing the hemiacetal hydroxyl group from the cyclic form of a monosaccharide or disaccharide. The monosaccharide or monosaccharides units can be selected from any 5-9 carbon atom containing sugars consisting of aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglycosamine, N-acetylmannosamine, N-acetylgalactosamine, etc.), uronic acids, ketoaldonic acids (e.g. sialic acid) and like.

The term “nitro” as used herein, means a —NO₂ group.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term, unless stated otherwise.

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

In accordance with the present disclosure, biosynthetic methods for synthesizing dihydrochalcones and/or dihydrostilbenoids are disclosed. Also, in accordance with the present disclosure nucleic acid constructs and recombinant cells which find use in the biosynthetic methods are provided.

### Recombinant Host Cells

In one aspect, the disclosure provides recombinant host cells having increased capacity to carry out reduction of an enoyl double bond of a phenylpropanoid to a dihydrophenylpropanoid. For example, in some embodiments the recombinant hosts have increased capacity to carry out reduction of the double bond of p-coumaroyl-CoA to dihydrocoumaroyl-CoA, and/or to carry out reduction of the double bond of cinnamoyl-CoA to dihydrocinnamoyl-CoA. In some instances, reduction of an enoyl double bond is carried out by an enoyl reductase (ENR). In other instances, reduction of an enoyl double bond is carried out by a polyprenol reductase. These reductases are also referred to collectively as double-bond reductases (DBRs). Thus, DBRs are a class of reductases that includes, inter alia, enoyl reductases (ENRs) and polyprenol reductases.

The increase in enoyl double bond reduction capacity may be attained by engineering the host cells to feature one or more heterologous recombinant enoyl reductase gene(s). In a first set of exemplary instances, the recombinant gene encodes an ENR enzyme having a percent amino acid sequence identity to the polypeptide of SEQ ID NO: 1 of at least 80%, e.g., at least 85%, at least 90%, at least 91%, at least 92%, at least 93%, at least 94%, at least 95%, at least 96%, at least 97%, at least 98%, at least 99%, or 100%. Also contemplated are instances where the recombinant ENR differs by no more than 50 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49, from the polypeptide of SEQ ID NO: 1. In preferred embodiments, the ENR differs by no more than 10 amino acids, e.g., 1, 2, 3, 4, 5, 6, 7, 8, 9 or 10 amino acids, from the polypeptide of SEQ ID NO: 1. In particularly preferred embodiments, the ENR comprises or consists of the amino acid sequence of SEQ ID NO: 1 or an allelic variant thereof; or is a fragment thereof having ENR activity.

In a second set of exemplary instances, the recombinant ENR has a percent amino acid sequence identity to the polypeptide of SEQ ID NO: 3 of at least 80%, e.g., at least 85%, at least 90%, at least 91%, at least 92%, at least 93%, at least 94%, at least 95%, at least 96%, at least 97%, at least 98%, at least 99%, or 100%. Also contemplated are instances where the ENR differs by no more than 50 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49, from the polypeptide of SEQ ID NO: 11. In preferred embodiments, the ENR differs by no more than 10 amino acids, e.g., 1, 2, 3, 4, 5, 6, 7, 8, 9 or 10 amino acids, from the polypeptide of SEQ ID NO: 3. In particularly preferred embodiments, the ENR comprises or consists of the amino acid sequence of SEQ ID NO: 3 or an allelic variant thereof; or is a fragment thereof having ENR activity.

In a third set of exemplary instances, the ENR has a percent amino acid sequence identity to the polypeptide of SEQ ID NO: 5 of at least 80%, e.g., at least 85%, at least 90%, at least 91%, at least 92%, at least 93%, at least 94%, at least 95%, at least 96%, at least 97%, at least 98%, at least 99%, or 100%. Also contemplated are instances where the ENR differs by no more than 50 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49, from the polypeptide of SEQ ID NO: 5. In preferred embodiments, the polypeptides differ by no more than 10 amino acids, e.g., 1, 2, 3, 4, 5, 6, 7, 8, 9 or 10 amino acids, from the polypeptide of SEQ ID NO: 5. In particularly preferred embodiments, the UDP-glycosyltransferase comprises or consists of the amino acid sequence of SEQ ID NO: 5 or an allelic variant thereof; or is a fragment thereof having ENR activity.

In a fourth set of exemplary instances, the ENR has a percent amino acid sequence identity to the polypeptide of SEQ ID NO: 7 of at least 80%, e.g., at least 85%, at least 90%, at least 91%, at least 92%, at least 93%, at least 94%, at least 95%, at least 96%, at least 97%, at least 98%, at least 99%, or 100%. Also contemplated are instances where the ENR differs by no more than 50 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49, from the polypeptide of SEQ ID NO: 7. In preferred embodiments, the polypeptides differ by no more than 10 amino acids, e.g., 1, 2, 3, 4, 5, 6, 7, 8, 9 or 10 amino acids, from the polypeptide of SEQ ID NO: 5. In particularly preferred embodiments, the ENR comprises or consists of the amino acid sequence of SEQ ID NO: 7 or an allelic variant thereof; or is a fragment thereof having ENR activity.

The recombinant host cells may co-express, along with the enoyl reductase, a recombinant polyketide synthase Type III polypeptide. In some embodiments, the recombinant polyketide synthase Type III polypeptide comprises: (i) a recombinant chalcone synthase (CHS) polypeptide; or (ii) a recombinant stilbene synthase (STS) polypeptide.

The recombinant host cells may be engineered to further express one or more polypeptides of a dihydrophenylpropanoid derivative biosynthesis pathway. In some embodiments, recombinant genes are provided that catalyze formation of intermediates in dihydrochalcone or dihydrostilbene biosynthesis. Intermediates may comprise, inter alia, cinnamic acid, cinnamoyl-CoA, dihydrocinnamoyl-CoA, p-coumaric acid, p-coumaroyl CoA, p-dihydrocoumaroyl CoA, and phloretin.

The recombinant enzyme may catalyze the formation of p-coumaroyl-CoA and/or cinnamoyl-CoA. Accordingly, in certain embodiments, the host cells express one or more recombinant gene encoding a 4-coumarate-CoA (4CL) ligase polypeptide, for example A. thaliana enzyme At4CL1 of SEQ ID NO: 11. In a representative example, the 4-coumarate-CoA ligase gene comprises SEQ ID NO: 12. In some embodiments, the 4-coumarate-CoA ligase gene has at least 70% identity to SEQ ID NO: 12, e.g., at least 75%, at least 80%, at least 85%, at least 90%, at least 91%, at least 92%, at least 93%, at least 94%, at least 95%, at least 96%, at least 97%, at least 98%, at least 99%, or 100%. Another suitable 4-coumarate-CoA (4CL) ligase polypeptide is provided by A. thaliana enzyme At4CL2 of SEQ ID NO: 13. In a representative example, the 4-coumarate-CoA ligase gene comprises SEQ ID NO: 14. In some embodiments, the 4-coumarate-CoA ligase gene has at least 70% identity to SEQ ID NO: 14, e.g., at least 75%, at least 80%, at least 85%, at least 90%, at least 91%, at least 92%, at least 93%, at least 94%, at least 95%, at least 96%, at least 97%, at least 98%, at least 99%, or 100%.

The recombinant cells may further comprise an endogenous or recombinant gene encoding a phenylalanine ammonia lyase (PAL) polypeptide, which catalyzes the formation of cinnamic acid. In some examples, the recombinant host cells express Arabidopsis thaliana AtPAL2 of SEQ ID NO: 15. In other examples, the recombinant host cells express recombinant gene AtPAL2 comprising the sequence disclosed herein as SEQ ID NO: 16. In other embodiments, the recombinant host cells express a recombinant gene with at least 70% identity to SEQ ID NO: 16, e.g., at least 75%, at least 80%, at least 85%, at least 90%, at least 91%, at least 92%, at least 93%, at least 94%, at least 95%, at least 96%, at least 97%, at least 98%, at least 99%, or 100%.

The host cells may also express a recombinant gene encoding a cinnamate 4-hydroxylase (C4H) polypeptide. The recombinant cinnamate 4-hydroxylase may be A. thaliana AtC4H including the polypeptide of SEQ ID NO: 17 which may be encoded by a gene comprising SEQ ID NO: 18. In some embodiments, the cinnamate 4-hydroxylase gene has at least 70% identity to SEQ ID NO: 18, e.g., at least 75%, at least 80%, at least 85%, at least 90%, at least 91%, at least 92%, at least 93%, at least 94%, at least 95%, at least 96%, at least 97%, at least 98%, at least 99%, or 100%.

The present disclosure also provides recombinant host cells engineered to express recombinant polypeptides that catalyze the formation of dihydrostilbenoids from p-dihydrocoumaroyl-CoA or dihydrocinnamoyl-CoA. Thus, the recombinant host cells may further comprise one or more stilbene synthase (STS) genes. In a representative example, the recombinant host cells express a gene coding for Vitis vinifera VvSTS (SEQ ID NO: 19). In some embodiments, the recombinant host cells express a recombinant gene comprising a genetic sequence of SEQ ID NO: 20. In further embodiments, the recombinant host cells express a recombinant gene with at least 70% identity to SEQ ID NO: 20, e.g., at least 75%, at least 80%, at least 85%, at least 90%, at least 91%, at least 92%, at least 93%, at least 94%, at least 95%, at least 96%, at least 97%, at least 98%, at least 99%, or 100%. In some embodiments, the recombinant host cells express a recombinant gene comprising a genetic sequence of SEQ ID NO: 21. In further embodiments, the recombinant host cells express a recombinant gene with at least 70% identity to SEQ ID NO: 21, e.g., at least 75%, at least 80%, at least 85%, at least 90%, at least 91%, at least 92%, at least 93%, at least 94%, at least 95%, at least 96%, at least 97%, at least 98%, at least 99%, or 100%. In some embodiments, the recombinant host cells express a recombinant gene comprising a genetic sequence of SEQ ID NO: 22. In further embodiments, the recombinant host cells express a recombinant gene with at least 70% identity to SEQ ID NO: 22, e.g., at least 75%, at least 80%, at least 85%, at least 90%, at least 91%, at least 92%, at least 93%, at least 94%, at least 95%, at least 96%, at least 97%, at least 98%, at least 99%, or 100%. In some embodiments, the recombinant host cells express a recombinant gene comprising a genetic sequence of SEQ ID NO: 23. In further embodiments, the recombinant host cells express a recombinant gene with at least 70% identity to SEQ ID NO: 23, e.g., at least 75%, at least 80%, at least 85%, at least 90%, at least 91%, at least 92%, at least 93%, at least 94%, at least 95%, at least 96%, at least 97%, at least 98%, at least 99%, or 100%. In some embodiments, the recombinant host cells express a recombinant gene comprising a genetic sequence of SEQ ID NO: 24. In further embodiments, the recombinant host cells express a recombinant gene with at least 70% identity to SEQ ID NO: 24, e.g., at least 75%, at least 80%, at least 85%, at least 90%, at least 91%, at least 92%, at least 93%, at least 94%, at least 95%, at least 96%, at least 97%, at least 98%, at least 99%, or 100%. In some embodiments, the recombinant host cells express a recombinant gene comprising a genetic sequence of SEQ ID NO: 25. In further embodiments, the recombinant host cells express a recombinant gene with at least 70% identity to SEQ ID NO: 25, e.g., at least 75%, at least 80%, at least 85%, at least 90%, at least 91%, at least 92%, at least 93%, at least 94%, at least 95%, at least 96%, at least 97%, at least 98%, at least 99%, or 100%.

The recombinant host cells may be further engineered to reduce or eliminate the activity of one or more endogenous ENR enzymes, for example by reducing or eliminating expression of one or more endogenous ENR genes. In a representative example, the endogenous enoyl reductase comprises Saccharomyces cerevisiae trans-2-enoyl-CoA reductase (TSC13, amino acid SEQ ID NO: 9), or a functional homolog thereof. In some embodiments, the endogenous enoyl reductase is encoded by a gene comprising the sequence disclosed herein as SEQ ID NO: 10. In further, non-exclusive embodiments, the endogenous enoyl reductase has a percent amino acid sequence identity to the polypeptide of SEQ ID NO: 9 of at least 80%, e.g., at least 85%, at least 90%, at least 91%, at least 92%, at least 93%, at least 94%, at least 95%, at least 96%, at least 97%, at least 98%, at least 99%, or 100%. Also contemplated are instances where the endogenous ENR differs by no more than 50 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49, from the polypeptide of SEQ ID NO: 9. In preferred embodiments, the endogenous polypeptide differ by no more than 10 amino acids, e.g., 1, 2, 3, 4, 5, 6, 7, 8, 9 or 10 amino acids, from the polypeptide of SEQ ID NO: 9. In particularly preferred embodiments, the endogenous ENR comprises or consists of the amino acid sequence of SEQ ID NO: 9 or an allelic variant thereof; or is a fragment thereof having ENR activity.

As used herein, “reduced expression” refers to expression of a gene or protein at a level lower than the native expression of the gene or protein. For example, in some embodiments the activity of a reductase is reduced by decreasing the amount of protein product, or expression, of a gene encoding the reductase.

Reduction or elimination (i.e., disruption) of expression of a gene can be accomplished by any known method, including insertions, missense mutations, frame shift mutations, deletion, substitutions, or replacement of a DNA sequence, or any combinations thereof. Insertions include the insertion of the entire genes, which may be of any origin. Reduction or elimination of gene expression can, for example, comprise altering or replacing a promoter, an enhancer, or splice site of a gene, leading to inhibition of production of the normal gene product partially or completely. In some embodiments, reduction or elimination of gene expression comprises altering the total level of the protein product expressed in the cell or organism. In other embodiments, disruption of a gene comprises reducing or eliminating the activity of the protein product of the gene in a cell or organism. In some embodiments of the disclosure, the disruption is a null disruption, wherein there is no significant expression of the gene. In some embodiments the disruption of a gene in a host cell or organism occurs on both chromosomes, in which case it is a homozygous disruption. In other embodiments the disruption of a gene in a host cell or organism occurs on only one chromosome, leaving the other chromosomal copy intact, in which case it is a heterozygous gene disruption. In still other embodiments each copy of a gene in a host cell or organism is disrupted differently.

Reduction or elimination of gene expression may also comprise gene knock-out or knock-down. A “gene knock-out” refers to a cell or organism in which the expression of one or more genes is eliminated. A “gene knock-down” refers to a cell or organism in which the level of one or more genes is reduced, but not completely eliminated.

In some embodiments, expression of a gene is reduced or eliminated by techniques such as RNA interference (RNAi), a process by which RNA molecules are used to inhibit gene expression, typically by causing destruction of specific mRNA molecules. RNAi is also known as co-suppression, post-transcriptional gene silencing (PTGS), and quelling.

As used herein, “reduced activity” refers to activity of a polypeptide, such as, for example, an enzyme, at a level lower than the native activity level of the polypeptide. Any means of reducing activity of a polypeptide can be used in the disclosed embodiments. For example, the sequence or the structure of the double-bond reductase may be altered, resulting in lower activity towards the original substrates of the enzyme. In another example, the activity of a double-bond reductase polypeptide may be reduced by growing a host cell in the presence of an inhibitor of the double-bond reductase polypeptide, or by co-expressing or co-producing an inhibitor of the double-bond reductase polypeptide.

### Recombinant Nucleic Acid Sequences

In a related aspect, the present disclosure also provides nucleic acid constructs comprising a nucleic acid sequence that encodes at least an ENR enzyme as described herein, as well as recombinant host cells comprising said nucleic acid construct(s). A recombinant gene encoding a polypeptide described herein comprises the coding sequence for that polypeptide, operably linked in sense orientation to one or more regulatory regions suitable for expressing the polypeptide. Because many hosts are capable of expressing multiple gene products from a polycistronic mRNA, multiple polypeptides can be expressed under the control of a single regulatory region for those hosts, if desired. A coding sequence and a regulatory region are considered operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In a first set of exemplary instances, the recombinant gene encodes an ENR enzyme having a percent amino acid sequence identity to the polypeptide of SEQ ID NO: 1 of at least 80%, e.g., at least 85%, at least 90%, at least 91%, at least 92%, at least 93%, at least 94%, at least 95%, at least 96%, at least 97%, at least 98%, at least 99%, or 100%. Also contemplated are instances where the recombinant ENR differs by no more than 50 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49, from the polypeptide of SEQ ID NO: 1. In preferred embodiments, the ENR differs by no more than 10 amino acids, e.g., 1, 2, 3, 4, 5, 6, 7, 8, 9 or 10 amino acids, from the polypeptide of SEQ ID NO: 1. In particularly preferred embodiments, the ENR comprises or consists of the amino acid sequence of SEQ ID NO: 1 or an allelic variant thereof; or is a fragment thereof having ENR activity.

In a second set of exemplary instances, the recombinant gene encodes an ENR enzyme having a percent amino acid sequence identity to the polypeptide of SEQ ID NO: 3 of at least 80%, e.g., at least 85%, at least 90%, at least 91%, at least 92%, at least 93%, at least 94%, at least 95%, at least 96%, at least 97%, at least 98%, at least 99%, or 100%. Also contemplated are instances where the ENR differs by no more than 50 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49, from the polypeptide of SEQ ID NO: 11. In preferred embodiments, the ENR differs by no more than 10 amino acids, e.g., 1, 2, 3, 4, 5, 6, 7, 8, 9 or 10 amino acids, from the polypeptide of SEQ ID NO: 3. In particularly preferred embodiments, the ENR comprises or consists of the amino acid sequence of SEQ ID NO: 3 or an allelic variant thereof; or is a fragment thereof having ENR activity.

In a third set of exemplary instances, the recombinant gene encodes an ENR enzyme having a percent amino acid sequence identity to the polypeptide of SEQ ID NO: 5 of at least 80%, e.g., at least 85%, at least 90%, at least 91%, at least 92%, at least 93%, at least 94%, at least 95%, at least 96%, at least 97%, at least 98%, at least 99%, or 100%. Also contemplated are instances where the ENR differs by no more than 50 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49, from the polypeptide of SEQ ID NO: 5. In preferred embodiments, the polypeptides differ by no more than 10 amino acids, e.g., 1, 2, 3, 4, 5, 6, 7, 8, 9 or 10 amino acids, from the polypeptide of SEQ ID NO: 5. In particularly preferred embodiments, the UDP-glycosyltransferase comprises or consists of the amino acid sequence of SEQ ID NO: 5 or an allelic variant thereof; or is a fragment thereof having ENR activity.

In a fourth set of exemplary instances, the recombinant gene encodes an ENR enzyme having a percent amino acid sequence identity to the polypeptide of SEQ ID NO: 7 of at least 80%, e.g., at least 85%, at least 90%, at least 91%, at least 92%, at least 93%, at least 94%, at least 95%, at least 96%, at least 97%, at least 98%, at least 99%, or 100%. Also contemplated are instances where the ENR differs by no more than 50 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49, from the polypeptide of SEQ ID NO: 7. In preferred embodiments, the polypeptides differ by no more than 10 amino acids, e.g., 1, 2, 3, 4, 5, 6, 7, 8, 9 or 10 amino acids, from the polypeptide of SEQ ID NO: 5. In particularly preferred embodiments, the ENR comprises or consists of the amino acid sequence of SEQ ID NO: 7 or an allelic variant thereof; or is a fragment thereof having ENR activity.

A nucleic acid construct may include a polynucleotide comprising a nucleotide sequence selected from the group consisting of a first sequence, a second sequence, a third sequence, and a fourth sequence. The first sequence has at least 80%, e.g., at least 85%, at least 90%, at least 91%, at least 92%, at least 93%, at least 94%, at least 95%, at least 96%, at least 97%, at least 98%, at least 99%, or 100% identity to the nucleotide sequence as set forth in SEQ ID NO: 2; the second sequence has at least 80%, e.g., at least 85%, at least 90%, at least 91%, at least 92%, at least 93%, at least 94%, at least 95%, at least 96%, at least 97%, at least 98%, at least 99%, or 100% identity to the nucleotide sequence as set forth in SEQ ID NO: 4; the third sequence has at least 80%, e.g., at least 85%, at least 90%, at least 91%, at least 92%, at least 93%, at least 94%, at least 95%, at least 96%, at least 97%, at least 98%, at least 99%, or 100% identity to the nucleotide sequence as set forth in SEQ ID NO: 6; the fourth sequence has at least 80%, e.g., at least 85%, at least 90%, at least 91%, at least 92%, at least 93%, at least 94%, at least 95%, at least 96%, at least 97%, at least 98%, at least 99%, or 100% identity to the nucleotide sequence as set forth in SEQ ID NO: 8.

In many cases, the coding sequence for a polypeptide described herein is identified in a species other than the recombinant host, i.e., is a heterologous nucleic acid. Thus, if the recombinant host is a microorganism, the coding sequence can be from other prokaryotic or eukaryotic microorganisms, from plants or from animals. In some case, however, the coding sequence is a sequence that may be native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. “Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also can include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region can be present, e.g., introns, enhancers, upstream activation regions, transcription terminators, and inducible elements.

### Cell Transformation

As stated above, a nucleic acid molecule encoding at least an ENR as described herein may be inserted into a host species, e.g., a bacterium or yeast, for example in the form of an expression vector. Hence, provided herein is a recombinant bacterial or yeast cell comprising a transgenic polynucleotide encoding a polypeptide selected from the ENRs disclosed herein.

Typically, the expression vector includes those genetic elements for expression of recombinant polypeptide(s) in host cells. The elements for transcription and translation in the host cell can include a promoter, a coding region for the protein complex, and a transcriptional terminator.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR). In a representative embodiment, an UGT-encoding sequences is inserted into a pETite plasmid vector (Lucigen, WI), to construct a pETite expression vector.

Several molecular biology techniques can be developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In some embodiments, synthetic linkers containing one or more restriction sites are used to operably link the polynucleotide(s) of the subject technology to the expression vector. In a non-exclusive embodiment, the polynucleotide is generated by restriction endonuclease digestion. In one technique, the nucleic acid molecule is treated with bacteriophage T4 DNA polymerase or E. coli DNA polymerase I, enzymes that remove protruding, 3′-single-stranded termini with their 3′-5′-exonucleolytic activities and fill in recessed 3′-ends with their polymerizing activities, thereby generating blunt-ended DNA segments. The blunt-ended segments are then incubated with a large molar excess of linker molecules in the presence of an enzyme that can catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun, et al, BIOTECHNIQUES 13, 515-18 (1992), which is incorporated herein by reference to the extent it is consistent herewith).

In some embodiments, to isolate and/or modify the polynucleotide(s) of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

A polynucleotide for incorporation into an expression vector of the subject technology may be prepared using PCR using appropriate oligonucleotide primers. The coding region is amplified, while the primers themselves become incorporated into the amplified sequence product. In some embodiments, the amplification primers contain restriction endonuclease recognition sites, which allow the amplified sequence product to be cloned into an appropriate vector.

The expression vectors can be introduced into the bacterial cells (e.g., E. coli) by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, bacterial cells (e.g., E. coli) transfected with an expression vector of the subject technology can be cultured to produce polypeptides described herein. Cells can be examined for the presence of the expression vector DNA by techniques well known in the art. The host cells can contain a single copy of the expression vector described previously, or alternatively, multiple copies of the expression vector.

In some embodiments, the nucleic acid molecule (e.g., vector) inserted in the bacterial cell further comprises a polynucleotide encoding a selection marker. A “selection marker” is a gene introduced into a cell, especially cells in culture, that confers a trait suitable for artificial selection. In some embodiments, a selectable marker is a gene that confers resistance to a drug to eukaryotic cells, including but not limited to kanamycin, paromomycin, puromycin, hygromycin, G418, neomycin, or bleomycin. In some embodiments, a selectable marker is a gene that confers resistance to kanamycin.

### Recombinant Hosts

Recombinant hosts can be used to express polypeptides for phenylpropanoid derivative or dihydrophenylpropanoid derivative production, including mammalian, insect, plant, and algal cells. A number of microbial prokaryotes and eukaryotes are also suitable for use in constructing the recombinant microorganisms described herein, e.g., gram-negative bacteria, yeast, and fungi. A species and strain selected for use as a phenylpropanoid derivative or dihydrophenylpropanoid derivative production strain is first analyzed to determine which production genes are endogenous to the strain and which genes are not present. Genes for which an endogenous counterpart is not present in the strain are advantageously assembled in one or more recombinant constructs, which are then transformed into the strain in order to supply the missing function(s).

The constructed and genetically engineered microorganisms provided herein can be cultivated using conventional fermentation processes, including, inter alia, chemostat, batch, fed-batch cultivations, continuous perfusion fermentation, and continuous perfusion cell culture.

Carbon sources of use include any molecule that can be metabolized by the recombinant host cell to facilitate growth and/or production of the dihydrophenylpropanoid derivative(s). Examples of suitable carbon sources include, but are not limited to, sucrose (e.g., as found in molasses), fructose, xylose, ethanol, glycerol, glucose, cellulose, starch, cellobiose or other glucose comprising polymer. In embodiments employing yeast as a host, for example, carbon sources such as sucrose, fructose, xylose, ethanol, glycerol, and glucose are suitable. The carbon source can be provided to the host organism throughout the cultivation period or alternatively, the organism can be grown for a period of time in the presence of another energy source, e.g., protein, and then provided with a source of carbon only during the fed-batch phase.

Exemplary prokaryotic and eukaryotic species are described in more detail below. However, it will be appreciated that other species can be suitable. For example, suitable species can be in a genus such as Agaricus, Aspergillus, Bacillus, Candida, Corynebacterium, Eremothecium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces or Yarrowia. Exemplary species from such genera include Lentinus tigrinus, Laetiporus sulphureus, Phanerochaete chrysosporium, Pichia pastoris, Cyberlindnera jadinii, Physcomitrella patens, Rhodoturula glutinis 32, Rhodoturula mucilaginosa, Phaffia rhodozyma UBV-AX, Xanthophyllomyces dendrorhous, Fusarium fujikuroi/Gibberella fujikuroi, Candida utilis, Candida glabrata, Candida albicans, and Yarrowia lipolytica.

In some embodiments, a microorganism can be a prokaryote such as Escherichia coli, Rhodobacter sphaeroides, Rhodobacter capsulatus, or Rhodotorula toruloides.

In some embodiments, a microorganism can be an Ascomycete such as Gibberella fujikuroi, Kiuyveromyces lactis, Schizosaccharomyces pombe, Aspergillus niger, Yarrowia lipolytica, Ashbya gossypii, or Saccharomyces cerevisiae.

In some embodiments, a microorganism can be an algal cell such as Blakeslea trispora, Dunaliella sauna, Haematococcus piuvialis, Chlorella sp., Undaria pinnatifida, Sargassum, Laminaria japonica, Scenedesmus almeriensis species.

In some embodiments, a microorganism can be a cyanobacterial cell such as Blakeslea trispora, Dunaliella sauna, Haematococcus pluvialis, Chlorella sp., Undaria pinnatifida, Sargassum, Laminaria japonica, Scenedesmus aimeriensis.

### Saccharomyces Spp.

Saccharomyces is a widely used chassis organism in synthetic biology, and can be used as the recombinant microorganism platform. For example, there are libraries of mutants, plasmids, detailed computer models of metabolism and other information available for S. cerevisiae, allowing for rational design of various modules to enhance product yield. Methods are known for making recombinant microorganisms.

### Aspergillus Spp.

Aspergillus species such as A. oryzae, A. niger and A. sojae are widely used microorganisms in food production and can also be used as the recombinant microorganism platform. Nucleotide sequences are available for genomes of A. nidulans, A. fumigatus, A. oryzae, A. ciavatus, A. flavus, A. niger, and A. terreus, allowing rational design and modification of endogenous pathways to enhance flux and increase product yield. Metabolic models have been developed for Aspergillus. Generally, A. niger is cultured for the industrial production of a number of food ingredients such as citric acid and gluconic acid, and thus species such as A. niger are generally suitable for producing phenylpropanoid derivatives or dihydrophenylpropanoid derivatives.

### Escherichia Coli

Escherichia coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

### Agaricus, Gibberella, and Phanerochaete Spp.

Agaricus, Gibberella, and Phanerochaete spp. can be useful because they are known to produce large amounts of isoprenoids in culture. Thus, precursors for producing large amounts of phenylpropanoid derivatives or dihydrophenylpropanoid derivatives are already produced by endogenous genes.

### Arxula Adeninivorans (Blastobotrys Adeninivorans)

Arxula adeninivorans is a dimorphic yeast (it grows as a budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

### Yarrowia Lipolytica

Yarrowia lipolytica is a dimorphic yeast (see Arxula adeninivorans) and belongs to the family Hemiascomycetes. The entire genome of Yarrowia lipolytica is known. Yarrowia species is aerobic and considered to be non-pathogenic. Yarrowia is efficient in using hydrophobic substrates (e.g. alkanes, fatty acids, oils) and can grow on sugars. It has a high potential for industrial applications and is an oleaginous microorganism. Yarrowia lipolyptica can accumulate lipid content to approximately 40% of its dry cell weight and is a model organism for lipid accumulation and remobilization. See e.g. Nicaud, 2012, Yeast 29(10):409-18; Beopoulos et al., 2009, Biohimie 91(6):692-6; Bankar et al., 2009, Appl Microbiol Biotechnol. 84(5):847-65.

### Rhodotorula Sp.

Rhodotorula is a unicellular, pigmented yeast. The oleaginous red yeast, Rhodotorula glutinis, has been shown to produce lipids and carotenoids from crude glycerol (Saenge et al., 2011, Process Biochemistry 46(1):210-8). Rhodotorula toruloides strains have been shown to be an efficient fed-batch fermentation system for improved biomass and lipid productivity (Li et al., 2007, Enzyme and Microbial Technology 41:312-7).

### Rhodosporidium Toruloides

Rhodosporidium toruloides is an oleaginous yeast and useful for engineering lipid-production pathways (See e.g. Zhu et al., 2013, Nature Commun. 3:1112; Ageitos et al., 2011, Applied Microbiology and Biotechnology 90(4):1219-27).

### Candida Boidinii

Candida boidinii is a methylotrophic yeast (it can grow on methanol). Like other methylotrophic species such as Hansenula polymorpha and Pichia pastoris, it provides an excellent platform for producing heterologous proteins. Yields in a multigram range of a secreted foreign protein have been reported. A computational method, IPRO, predicted mutations that experimentally switched the cofactor specificity of Candida boidinii xylose reductase from NADPH to NADH.

### Hansenula Polymorpha (Pichia Angusta)

Hansenula polymorpha is another methylotrophic yeast (see Candida boidinii). It can furthermore grow on a wide range of other substrates; it is thermo-tolerant and can assimilate nitrate (see also Kluyveromyces lactis). It has been applied to producing hepatitis B vaccines, insulin and interferon alpha-2a for the treatment of hepatitis C, furthermore to a range of technical enzymes.

### Kluyveromyces Lactis

Kluyveromyces lactis is yeast regularly applied to producing kefir. It can grow on several sugars, most importantly on lactose which is present in milk and whey. It has successfully been applied among others for producing chymosin (an enzyme that is usually present in the stomach of calves) for producing cheese. Production takes place in fermenters on a 40,000 L scale.

### Pichia Pastoris

Pichia pastoris is a methylotrophic yeast (see Candida boidinii and Hansenula polymorpha). It provides an efficient platform for producing foreign proteins. Platform elements are available as a kit and it is worldwide used in academia for producing proteins. Strains have been engineered that can produce complex human N-glycan (yeast glycans are similar but not identical to those found in humans).

### Physcomitrella Spp.

Physcomitrella mosses, when grown in suspension culture, have characteristics similar to yeast or other fungal cultures. This genera is becoming an important type of cell for producing plant secondary metabolites, which can be difficult to produce in other types of cells.

### Biosynthesis of Dihydrophenylpropanoid Derivative Compounds

In another aspect, the disclosure provides a method of producing a dihydrochalcone or a dihydrostilbene compound, comprising growing a recombinant host cell as disclosed herein in a culture medium under conditions in which the recombinant genes are expressed, and wherein said compound is synthesized by the recombinant host cell.

In a representative example, the method of the disclosure is used to produce a dihydrochalcone compound. In some embodiments, the dihydrochalcone compound is phloretin or a phloretin derivative. The phloretin derivative may be phlorizin.

In addition to phlorizin, the method disclosed herein is useful for producing other dihydrochalcones, e.g., neohesperidin dihydrochalcone (NHDC). In some embodiments, the method is applied in the production of a dihydrostilbenoid compound.

The method of producing a dihydrochalcone or a dihydrostilbene compound may further comprise harvesting the product compound. As used herein, the term “harvesting” refers to any means of collecting a compound, which may or may not comprise isolating the compound. In some embodiments, the method of producing a dihydrochalcone or a dihydrostilbene compound further comprise isolating said compound.

In representative examples, the product compound is a molecule of formula (V):

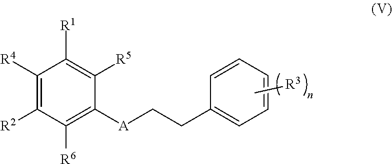

or a pharmaceutically acceptable salt thereof, wherein
A is a bond or C═O;
n is an integer 0, 1, 2, 3, or 4;
R¹ is hydrogen or —OR¹¹;
wherein each R¹¹ is independently hydrogen, C₁-C₆ alkyl, or glycosyl;
R² is hydrogen, —OR¹¹, C₁-C₁₂ alkyl, or C₂-C₁₂ alkenyl, wherein alkyl and alkenyl are optionally substituted with one or more R⁷;
   or R² and R⁶ together with the atoms to which they are attached form a 5- to 7-member heterocyclyl optionally substituted with one or more R⁷ groups;
   or R² and R⁴ together with the atoms to which they are attached form a 5- to 7-member heterocyclyl optionally substituted with one or more R⁷ groups;
R³ is independently selected from nitro, C₁-C₆ alkyl, C₂-C₆ alkenyl, C₁-C₆ haloalkyl, C₁-C₆ hydroxyalkyl, —OR¹², —N(R¹²)2, —C(O)R¹², —C(O)OR¹², —C(O)N(R¹²)₂, and —S(O)₂R¹², wherein each R¹² is independently hydrogen or C₁-C₆ alkyl;
R⁴ is hydrogen, —OR¹¹, C₁-C₁₂ alkyl, or C₂-C₁₂ alkenyl, wherein alkyl and alkenyl are optionally substituted with one or more R⁷;
   or R⁴ and R² together with the atoms to which they are attached form a 5- to 7-member heterocyclyl optionally substituted with one or more R⁷ groups;
R⁵ is hydrogen or —OR¹¹; and
R⁶ is hydrogen, C₁-C₆ alkyl, C₂-C₆ alkenyl, —OR¹¹, or —N(R¹⁰)₂, wherein each R¹⁰ is independently hydrogen or C₁-C₆ alkyl, and wherein alkyl and alkenyl are optionally substituted with one or more R⁸; or R⁶ and R² together with the atoms to which they are attached form a 5- to 7-member heterocyclyl optionally substituted with one or more R⁸ groups;
   each R⁷ and R⁸ is independently halogen, cyano, nitro, C₁-C₆ alkyl, C₂-C₆ alkenyl, C₁-C₆ haloalkyl, C₁-C₆ hydroxyalkyl, —OR¹³, —SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl,
   comprising growing a recombinant host cell as disclosed herein in a culture medium under conditions in which the recombinant genes are expressed, and wherein the compound of formula (V) is synthesized by the recombinant host cell.

In some embodiments, the compound of formula (V) is not a compound wherein R¹, R², and R⁴ are independently hydrogen.

The compound of formula (V) can be a dihydrostilbenoid compound, where A is a bond. In a representative example, the dihydrostilbenoids produced by the method of the invention include those of formula (V-A):

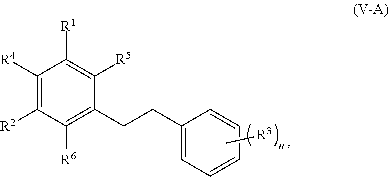

or a pharmaceutically acceptable salt thereof, wherein
n is an integer 0, 1, 2, 3, or 4;
R¹ is hydrogen or —OR¹¹;
wherein each R¹¹ is independently hydrogen, C₁-C₆ alkyl, or glycosyl;
R² is hydrogen, —OR¹¹, C₁-C₁₂ alkyl, or C₂-C₁₂ alkenyl, wherein alkyl and alkenyl are optionally substituted with one or more R⁷;
   or R² and R⁶ together with the atoms to which they are attached form a 5- to 7-member heterocyclyl optionally substituted with one or more R⁷ groups;
   or R² and R⁴ together with the atoms to which they are attached form a 5- to 7-member heterocyclyl optionally substituted with one or more R⁷ groups;
R³ is independently selected from nitro, C₁-C₆ alkyl, C₂-C₆ alkenyl, C₁-C₆ haloalkyl, C₁-C₆ hydroxyalkyl, —OR¹², —N(R¹²)2, —C(O)R¹², —C(O)OR¹², —C(O)N(R¹²)2, and —S(O)₂R¹², wherein each R¹² is independently hydrogen or C₁-C₆ alkyl;
R⁴ is hydrogen, —OR¹¹, C₁-C₁₂ alkyl, or C₂-C₁₂ alkenyl, wherein alkyl and alkenyl are optionally substituted with one or more R⁷;
   or R⁴ and R² together with the atoms to which they are attached form a 5- to 7-member heterocyclyl optionally substituted with one or more R⁷ groups;
R⁵ is hydrogen or —OR¹¹; and
R⁶ is hydrogen, C₁-C₆ alkyl, C₂-C₆ alkenyl, —OR¹¹, or —N(R¹⁰)₂, wherein each R¹⁰ is independently hydrogen or C₁-C₆ alkyl, and wherein alkyl and alkenyl are optionally substituted with one or more R⁸; or R⁶ and R² together with the atoms to which they are attached form a 5- to 7-member heterocyclyl optionally substituted with one or more R⁸ groups;
each R⁷ and R⁸ is independently halogen, cyano, nitro, C₁-C₆ alkyl, C₂-C₆ alkenyl, C₁-C₆haloalkyl, C₁-C₆ hydroxyalkyl, —OR¹³, —SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl.

In some embodiments, compounds of formula (V-A) are those wherein:
n is an integer 0, 1, 2, 3, or 4;
R¹ is hydrogen or —OR¹¹;
R² is hydrogen, —OR¹¹, C₁-C₁₂ alkyl, or C₂-C₁₂ alkenyl, wherein alkyl and alkenyl are optionally substituted with one or more R⁷;
   or R² and R⁶ together with the atoms to which they are attached form a 5- to 7-member heterocyclyl optionally substituted with one or more R⁷ groups;
   or R² and R⁴ together with the atoms to which they are attached form a 5- to 7-member heterocyclyl optionally substituted with one or more R⁷ groups;
R³ is independently selected from nitro, C₁-C₆ alkyl, C₂-C₆ alkenyl, C₁-C₆ haloalkyl, C₁-C₆ hydroxyalkyl, —OR¹², —N(R¹²)₂, —C(O)R¹², —C(O)OR¹², —C(O)N(R¹²)₂, and —S(O)₂R¹², wherein each R¹² is independently hydrogen or C₁-C₆ alkyl;
R⁴ is —OR¹¹, C₁-C₁₂ alkyl or C₂-C₁₂ alkenyl, wherein alkyl and alkenyl are optionally substituted with one or more R⁷;
   or R⁴ and R² together with the atoms to which they are attached form a 5- to 7-member heterocyclyl optionally substituted with one or more R⁷ groups;
R⁵ is hydrogen or —OR¹¹; and
R⁶ is hydrogen, C₁-C₆ alkyl, C₂-C₆ alkenyl, —OR¹¹, or —N(R¹⁰)₂, wherein each R¹⁰ is independently hydrogen or C₁-C₆ alkyl, and wherein alkyl and alkenyl are optionally substituted with one or more R⁸; or R⁶ and R² together with the atoms to which they are attached form a 5- to 7-member heterocyclyl optionally substituted with one or more R⁸ groups;
   each R⁷ and R⁸ is independently halogen, cyano, nitro, C₁-C₆ alkyl, C₂-C₆ alkenyl, C₁-C₆ haloalkyl, C₁-C₆ hydroxyalkyl, —OR¹³, —SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl.

In some examples, compounds of formula (V-A) are those wherein:
n is an integer 0, 1, 2, 3, or 4;
R¹ is —OR¹¹;
R² is —OR¹¹, C₁-C₁₂ alkyl, or C₂-C₁₂ alkenyl, wherein alkyl and alkenyl are optionally substituted with one or more R⁷; wherein R¹¹ is independently hydrogen or C₁-C₆ alkyl;
   or R² and R⁶ together with the atoms to which they are attached form a 5- to 7-member heterocyclyl optionally substituted with one or more R⁷ groups;
   or R² and R⁴ together with the atoms to which they are attached form a 5- to 7-member heterocyclyl optionally substituted with one or more R⁷ groups;
R³ is independently selected from C₁-C₆ hydroxyalkyl, —OR¹², —N(R¹²)₂, —C(O)OR¹², and —C(O)N(R¹²)₂, wherein each R¹² is independently hydrogen or C₁-C₆ alkyl;
R⁴ is —OR¹¹ or C₂-C₁₂ alkenyl, wherein alkenyl is optionally substituted with one or more R⁷;
   or R⁴ and R² together with the atoms to which they are attached form a 5- to 7-member heterocyclyl optionally substituted with one or more R⁷ groups;
R⁵ is hydrogen; and
R⁶ is hydrogen or C₂-C₆ alkenyl, wherein alkenyl is optionally substituted with one or more R⁸; or R⁶ and R² together with the atoms to which they are attached form a 5- to 7-member heterocyclyl optionally substituted with one or more R⁸ groups;
   each R⁷ and R⁸ is independently halogen, cyano, nitro, C₁-C₆ alkyl, C₂-C₆ alkenyl, C₁-C₆ haloalkyl, C₁-C₆ hydroxyalkyl, —OR¹³, —SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl.

In some embodiments, compounds of formula (V-A) are those wherein n is 0. In other embodiments, compounds of formula (V-A) are those where R¹ is —OR¹¹, and R¹¹ is hydrogen or methyl. Some embodiments provide compounds of formula (V-A) where R¹ is hydrogen.

Some embodiments provide compounds of formula (V-A) where R² is —OR¹¹, and R¹¹ is independently hydrogen or C₁-C₆ alkyl. In some embodiments, R¹¹ is hydrogen or methyl. Other embodiments provide compounds of formula (V-A) where R² is hydrogen.

Some embodiments provide compounds of formula (V-A) where R⁴ is —OR¹¹, and R¹¹ is independently hydrogen or C₁-C₆ alkyl. In some embodiments, R¹¹ is hydrogen or methyl. Other embodiments provide compounds of formula (V-A) where R⁴ is C₂-C₁₂ alkenyl optionally substituted with one or more R⁷. In some embodiments, R⁴ is C₂-C₁₂ alkenyl optionally substituted with hydroxy. In some embodiments, R⁴ is 3-methylbut-2-en-1-yl optionally substituted with hydroxy. In some embodiments, R⁴ is 3-methylbut-2-en-1-yl.

Some embodiments provide compounds of formula (V-A) where R⁵ is hydrogen.

Some embodiments provide compounds of formula (V-A) where R⁶ is hydrogen.

Representative examples of compounds of formula (V-A) include, but are not limited to the following: dihydroresveratrol, dihydropinosylvin, and amorfrutin 2.

The compound of formula (V) can also be a dihydrochalcone compound of formula (V-B):

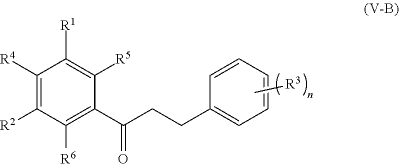

or a pharmaceutically acceptable salt thereof, wherein
n is an integer 0, 1, 2, 3, or 4;
R¹ is hydrogen or —OR¹¹;
wherein each R¹¹ is independently hydrogen, C₁-C₆ alkyl, or glycosyl;
R² is hydrogen, —OR¹¹, C₁-C₁₂ alkyl, or C₂-C₁₂ alkenyl, wherein alkyl and alkenyl are optionally substituted with one or more R⁷;
   or R² and R⁶ together with the atoms to which they are attached form a 5- to 7-member heterocyclyl optionally substituted with one or more R⁷ groups;
   or R² and R⁴ together with the atoms to which they are attached form a 5- to 7-member heterocyclyl optionally substituted with one or more R⁷ groups;
R³ is independently selected from nitro, C₁-C₆ alkyl, C₂-C₆ alkenyl, C₁-C₆ haloalkyl, C₁-C₆ hydroxyalkyl, —OR¹², —N(R¹²)2, —C(O)R¹², —C(O)OR¹², —C(O)N(R¹²)2, and —S(O)₂R¹², wherein each R¹² is independently hydrogen or C₁-C₆ alkyl;
R⁴ is hydrogen, —OR¹¹, C₁-C₁₂ alkyl, or C₂-C₁₂ alkenyl, wherein alkyl and alkenyl are optionally substituted with one or more R⁷;
   or R⁴ and R² together with the atoms to which they are attached form a 5- to 7-member heterocyclyl optionally substituted with one or more R⁷ groups;
R⁵ is hydrogen or —OR¹¹; and
R⁶ is hydrogen, C₁-C₆ alkyl, C₂-C₆ alkenyl, —OR¹¹, or —N(R¹⁰)₂, wherein each R¹⁰ is independently hydrogen or C₁-C₆ alkyl, and wherein alkyl and alkenyl are optionally substituted with one or more R⁸; or R⁶ and R² together with the atoms to which they are attached form a 5- to 7-member heterocyclyl optionally substituted with one or more R⁸ groups;
each R⁷ and R⁸ is independently halogen, cyano, nitro, C₁-C₆ alkyl, C₂-C₆ alkenyl, C₁-C₆haloalkyl, C₁-C₆ hydroxyalkyl, —OR¹³, —SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl.

In some embodiments, compounds of formula (V-B) are those wherein:
n is an integer 0, 1, 2, 3, or 4;
R¹ is hydrogen or —OR¹¹;
R² is hydrogen, —OR¹¹, C₁-C₁₂ alkyl, or C₂-C₁₂ alkenyl, wherein alkyl and alkenyl are optionally substituted with one or more R⁷;
   or R² and R⁶ together with the atoms to which they are attached form a 5- to 7-member heterocyclyl optionally substituted with one or more R⁷ groups;
   or R² and R⁴ together with the atoms to which they are attached form a 5- to 7-member heterocyclyl optionally substituted with one or more R⁷ groups;
R³ is independently selected from nitro, C₁-C₆ alkyl, C₂-C₆ alkenyl, C₁-C₆ haloalkyl, C₁-C₆ hydroxyalkyl, —OR¹², —N(R¹²)₂, —C(O)R¹², —C(O)OR¹², —C(O)N(R¹²)₂, and —S(O)₂R¹², wherein each R¹² is independently hydrogen or C₁-C₆ alkyl;
R⁴ is —OR¹¹, C₁-C₁₂ alkyl or C₂-C₁₂ alkenyl, wherein alkyl and alkenyl are optionally substituted with one or more R⁷;
   or R⁴ and R² together with the atoms to which they are attached form a 5- to 7-member heterocyclyl optionally substituted with one or more R⁷ groups;
R⁵ is hydrogen or —OR¹¹; and
R⁶ is hydrogen, C₁-C₆ alkyl, C₂-C₆ alkenyl, —OR¹¹, or —N(R¹⁰)₂, wherein each R¹⁰ is independently hydrogen or C₁-C₆ alkyl, and wherein alkyl and alkenyl are optionally substituted with one or more R⁸; or R⁶ and R² together with the atoms to which they are attached form a 5- to 7-member heterocyclyl optionally substituted with one or more R⁸ groups;
   each R⁷ and R⁸ is independently halogen, cyano, nitro, C₁-C₆ alkyl, C₂-C₆ alkenyl, C₁-C₆ haloalkyl, C₁-C₆ hydroxyalkyl, —OR¹³, —SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl.

In some embodiments, compounds of formula (V-B) are those wherein:
n is an integer 0, 1, 2, 3, or 4;
R¹ is —OR¹¹;
R² is —OR¹¹, C₁-C₁₂ alkyl, or C₂-C₁₂ alkenyl, wherein alkyl and alkenyl are optionally substituted with one or more R⁷; wherein R¹¹ is independently hydrogen or C₁-C₆ alkyl;
   or R² and R⁶ together with the atoms to which they are attached form a 5- to 7-member heterocyclyl optionally substituted with one or more R⁷ groups;
   or R² and R⁴ together with the atoms to which they are attached form a 5- to 7-member heterocyclyl optionally substituted with one or more R⁷ groups;
R³ is independently selected from C₁-C₆ hydroxyalkyl, —OR¹², —N(R¹²)₂, —C(O)OR¹², and —C(O)N(R¹²)₂, wherein each R¹² is independently hydrogen or C₁-C₆ alkyl;
R⁴ is —OR¹¹ or C₂-C₁₂ alkenyl, wherein alkenyl is optionally substituted with one or more R⁷;
   or R⁴ and R² together with the atoms to which they are attached form a 5- to 7-member heterocyclyl optionally substituted with one or more R⁷ groups;
R⁵ is hydrogen; and
R⁶ is hydrogen or C₂-C₆ alkenyl, wherein alkenyl is optionally substituted with one or more R⁸; or R⁶ and R² together with the atoms to which they are attached form a 5- to 7-member heterocyclyl optionally substituted with one or more R⁸ groups;
   each R⁷ and R⁸ is independently halogen, cyano, nitro, C₁-C₆ alkyl, C₂-C₆ alkenyl, C₁-C₆ haloalkyl, C₁-C₆ hydroxyalkyl, —OR^{13′} SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl.

In some embodiments, compounds of formula (V-B) are those wherein n is 0. In other embodiments, compounds of formula (V-B) are those where R¹ is —OR¹¹, and R¹¹ is hydrogen or methyl. Some embodiments provide compounds of formula (V-B) where R¹ is hydrogen.

Some embodiments provide compounds of formula (V-B) where R² is —OR¹¹, and R¹¹ is independently hydrogen or C₁-C₆ alkyl. In some embodiments, R¹¹ is hydrogen or methyl. Other embodiments provide compounds of formula (V-B) where R² is hydrogen.

Some embodiments provide compounds of formula (V-B) where R⁴ is —OR¹¹, and R¹¹ is independently hydrogen or C₁-C₆ alkyl. In some embodiments, R¹¹ is hydrogen or methyl. Other embodiments provide compounds of formula (V-B) where R⁴ is C₂-C₁₂ alkenyl optionally substituted with one or more R⁷. In some embodiments, R⁴ is C₂-C₁₂ alkenyl optionally substituted with hydroxy. In some embodiments, R⁴ is 3-methylbut-2-en-1-yl optionally substituted with hydroxy. In some embodiments, R⁴ is 3-methylbut-2-en-1-yl.

Some embodiments provide compounds of formula (V-B) where R⁵ is hydrogen.

Some embodiments provide compounds of formula (V-B) where R⁶ is hydrogen.

Representative examples of compounds of formula (V-B) include, but are not limited to phloretin, phlorizin, and pinocembrin dihydrochalcone.

The method of producing a compound of any one of formulae (V), (V-A), or (V-B) may further comprise harvesting the product compound. In some embodiments, the method of producing a compound of any one of formulae (V), (V-A), or (V-B) further comprises isolating said compound.

In a representative example, the method includes growing the recombinant host in a culture medium under conditions in which dihydrophenylpropanoid and dihydrophenylpropanoid derivative biosynthesis genes are expressed. The recombinant host can be grown in a fed batch or continuous process. Typically, the recombinant host is grown in a fermenter at a defined temperature(s) for a desired period of time. Depending on the particular host used in the method, other recombinant genes can also be present and expressed. Levels of substrates and intermediates can be determined by extracting samples from culture media for analysis according to published methods.

After the recombinant host has been grown in culture for the desired period of time, dihydrophenylpropanoid and/or their derivatives (such as phlorizin or phlorizin precursors) can then be recovered from the culture using various techniques known in the art. In some embodiments, a permeabilizing agent can be added to aid the feedstock entering into the host, and to aid in product release from the host. For example, a crude lysate of the cultured microorganism can be centrifuged to obtain a supernatant. The resulting supernatant can then be applied to a chromatography column, e.g., a C-18 column, and washed with water to remove hydrophilic compounds, followed by elution of the compound(s) of interest with a solvent such as methanol. The compound(s) can then be further purified by preparative HPLC according to methods known in the art.

It will be appreciated that the various genes discussed herein can be present in two or more recombinant hosts rather than a single host. When a plurality of recombinant host is used, they can be grown in a mixed culture to produce phenylpropanoids and/or dihydrophenylpropanoid and their respective derivatives.

Alternatively, the two or more hosts each can be grown in a separate culture medium and the product of the first culture medium, e.g., a phlorizin precursor, can be introduced into the second culture medium to be converted into a subsequent intermediate, or into an end product such as phlorizin. The product produced by the second, or final host is then recovered. It will also be appreciated that in some embodiments, a recombinant host is grown using nutrient sources other than a culture medium and utilizing a system other than a fermenter.

In one exemplary application, dihydrophenylpropanoid derivatives are produced in vivo through expression of one or more enzymes involved in a dihydrophenylpropanoid biosynthetic pathway in a recombinant host. For example, a phloretin-producing or dihydroresveratrol-producing recombinant host wherein one or more genes encoding a Saccharomyces cerevisiae trans-2-enoyl-CoA reductase polypeptide are underexpressed or unexpressed, and expressing recombinant genes encoding one or more transgenic trans-2-enoyl-CoA reductase polypeptide such that the production of dihydrophenylpropanoids and their derivatives is increased relative to the recombinant host where the one or more genes encoding a Saccharomyces cerevisiae trans-2-enoyl-CoA reductase polypeptide are not either underexpressed or unexpressed. The host may also express recombinant genes encoding one or more of an Arabidopsis thaliana phenylalanine ammonia lyase (AtPAL2) polypeptide, a gene encoding an Arabidopsis thaliana cinnamate 4-hydroxylase (AtCH4) polypeptide, a gene encoding an Arabidopsis thaliana 4-coumarate-CoA ligase (At4CL1 and/or At4CL2) polypeptide, a gene encoding chalcone synthase (CHS) polypeptide, and/or a gene encoding a cytochrome P450 reductase (CPR1) polypeptide can be used to produce a dihydrochalcone compound, e.g. phloretin, in vivo.

As another example, a dihydrostilbenoid (such as dihydroresveratrol)-producing recombinant host is provided wherein one or more genes encoding a Saccharomyces cerevisiae trans-2-enoyl-CoA reductase polypeptide (e.g., TSC13) are underexpressed or unexpressed, and expressing recombinant genes encoding one or more transgenic trans-2-enoyl-CoA reductase polypeptide such that the production of dihydrophenylpropanoid derivatives is increased relative to the recombinant host where the one or more genes encoding a Saccharomyces cerevisiae trans-2-enoyl-CoA reductase polypeptide are not either underexpressed or unexpressed.

In some embodiments, dihydrophenylpropanoid and their derivatives are produced through contact of a precursor of the desired compound with one or more enzymes involved a dihydrophenylpropanoid biosynthesis pathway in vitro. For example, contacting dihydrocoumaroyl-CoA with a chalcone synthase polypeptide can result in production of a phloretin or phloretin derivative compound in vitro. In some embodiments, a phloretin precursor is produced through contact of an upstream phloretin precursor with one or more enzymes involved in the phloretin pathway in vitro. As another example, contacting phloretin with a P2′UGT polypeptide can result in production of a phlorizin compound in vitro. In some embodiments, a phlorizin precursor is produced through contact of an upstream phlorizin precursor with one or more enzymes involved in the phlorizin pathway in vitro. As another example, contacting p-coumaroyl-CoA with a trans-2-enoyl-CoA reductase enzyme can result in production of dihydrocoumaroyl-CoA in vitro.

In further embodiments, a dihydrophenylpropanoid derivative is produced by bioconversion. For bioconversion to occur, a host cell expressing one or more enzymes involved in the dihydrophenylpropanoid derivative biosynthesis pathway takes up and modifies a dihydrophenylpropanoid derivative precursor in the cell; following modification in vivo, the dihydrophenylpropanoid derivative remains in the cell and/or is excreted into the culture medium. For example, a host cell expressing a gene encoding a chalcone synthase polypeptide can take up dihydrocoumaroyl CoA and convert it to phloretin in the cell; following conversion in vivo, a phloretin compound is excreted into the culture medium. As another example, a host cell expressing a gene encoding a UGT polypeptide can take up phloretin and glycosylate phloretin in the cell; following glycosylation in vivo, a phlorizin compound is excreted into the culture medium.

The phenylpropanoid derivatives or dihydrophenylpropanoid derivatives as disclosed herein may isolated and purified to homogeneity (e.g., at least 90%, 92%, 94%, 96%, or 98% pure). In some embodiments, phenylpropanoid derivatives or dihydrophenylpropanoid derivatives are isolated as an extract from a recombinant host or in vitro production method. In this respect, phenylpropanoid derivatives or dihydrophenylpropanoid derivatives may be isolated, but not necessarily purified to homogeneity. Desirably, the amount of phenylpropanoid derivatives or dihydrophenylpropanoid derivatives produced can be from about 1 mg/L to about 20,000 mg/L or higher. For example about 1 to about 100 mg/L, about 30 to about 100 mg/L, about 50 to about 200 mg/L, about 100 to about 500 mg/L, about 100 to about 1,000 mg/L, about 250 to about 5,000 mg/L, about 1,000 to about 15,000 mg/L, or about 2,000 to about 10,000 mg/L of phenylpropanoid derivatives or dihydrophenylpropanoid derivatives can be produced. In general, longer culture times will lead to greater amounts of product. Thus, the recombinant microorganism can be cultured for from 1 day to 7 days, from 1 day to 5 days, from 3 days to 5 days, about 3 days, about 4 days, or about 5 days.

### EXAMPLES

### Example 1: Construction of Background Strains for Bioconversion Process

The genome of S. cerevisiae strain BY4741 was modified by deletion of the Aro10 open reading frame. Aro10 is phenylpyruvate decarboxylase catalyzing phenylpyruvate degradation to phenylacetaldehyde. The gene was deleted by replacing Aro10 with the Met15 marker. Approximately 1000 base pairs of upstream and downstream flanking region of Aro10 coding sequences were amplified producing two PCR products. Complete gene sequence of Met15 including promoter and terminator was amplified separately. Those three PCR products, Aro10 upstream, Met15 and Aro10 downstream were stitched together by overlapped PCR to produce Aro10 knock out DNA fragment. The DNA fragment was transformed directly into BY4741 and selected for methionine prototrophy. The resulting strain was designated as CNFS004 and was used as a background strain for all resveratrol bioconversion strains.

The CNFS004 strain was further modified by integrating At4CL1, one of the resveratrol biosynthetic pathway genes. At4CL1 is one of four 4-coumaric acid:Coenzyme A ligase from Arabidopsis thaliana. The open reading frame of At4CL1 was codon optimized (SEQ ID NO: 12) and integrated into the PDC5 locus. PDC5 (amino acid SEQ ID NO: 26, DNA SEQ ID NO: 27) is another decarboxylase that degrades phenylpyruvate. The entire open reading frame of PDC5 was replaced with At4CL1 flanked by the PGK1 promoter and the SSAI terminator. The product strain was designated as CNFS007.

CNFS007 was engineered to produce resveratrol. The engineering was conducted by integrating one copy of gene for each steps including AtPAL2, AtC4H, At4CL2, and two copies of VvSTS. The sources for genes are described in Table 1.

TABLE 1
Cassettes to integrate resveratrol pathway gene cassettes into XII-1 locus

| | Assembler 1 | | Assembler 2 | | Assembler 3 | |
|---|---|---|---|---|---|---|
| ORF1 | VvSTS opt2 | Vitis | AtC4H- | Arabidopsis | pADH1- | Saccharomyces |
| | | vinifera | ATR2 | thaliana | ACC1 | cerevisiae |
| ORF2 | VvSTS opt5 | Vitis | AtPAL2 | Arabidopsis | At4CL2 | Arabidopsis |
| | | vinifera | | thaliana | | thaliana |

The resulting strain was designated as CNFS78. The strain was subjected to further modification by integrating multiple copies of genes described above. More than 2-3 copies of each of AtPAL, At4CL, AC4H and several copies of VvSTS were integrated by two iterations of transformation and the product strains were designated as CNFS171 and CNFS173.

Mutant Aro4K229L and Aro7G141S were integrated into CNFS173 to relieve feedback resistance by tyrosine or phenylalanine. The strains were designated as CNFS204.

One more transformation step integrated multiple copies of stilbene synthase VvSTS and one copy of feedback-resistant ACC1S659A, S1157A, to generated strain that was designated CNFS226 and cured for markers using a cre-loxP recombination system, yielding a strain that was designated CNFS273.

Further multiple copies of VvSTSs were integrated to improve resveratrol production to generate strain CNFS242 whose marker was cured yielding strain CNFS280.

CNFS273 produced less amount of resveratrol compared to CNFS280. Therefore, CNFS273 was designated as medium titer RSV parent and CNFS280 was designated as high titer RSV parent for TSC13 testing.

### Example 2. Testing TSC13 Homologs in Resveratrol-Producing Strains

In order to increases the production of dihydroresveratrol and phloretin, multiple TSC13 homologs were screened to find more active enzymes that are able to fully complement native TSC13 when it is replaced. Likely because TSC13 is an essential enzyme, strains expressing plant-derived MdECR (Malus domestica enoyl-ACP reductase) in S. cerevisiae showed growth impairment possibly because it failed to fully complement the function of native TSC13.

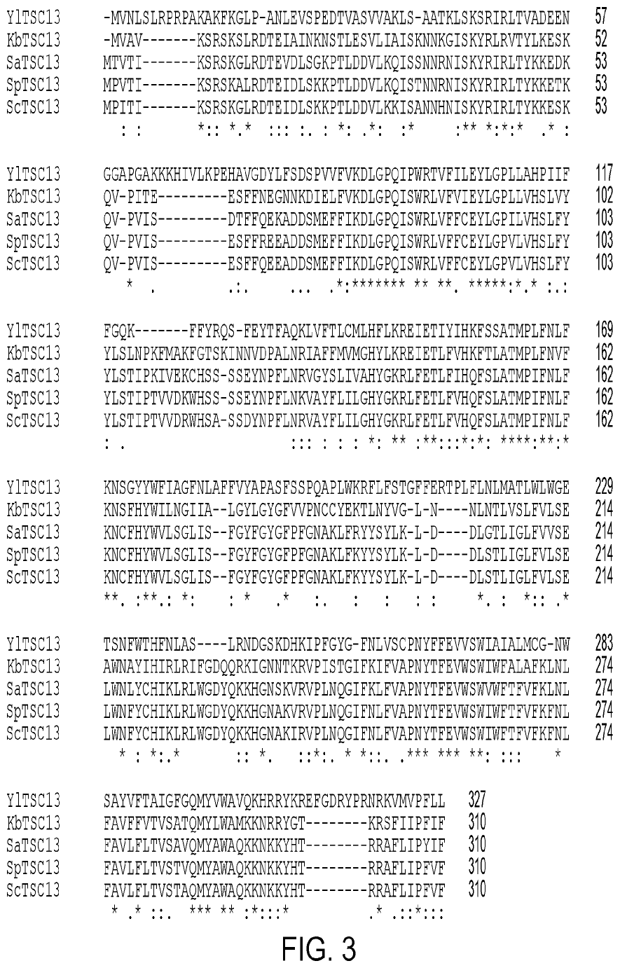

It was therefore theorized that TSC13 homologs from yeast species other than S. cerevisiae would be more likely to prove highly active while fully complementing native TSC13 without growth retardation. Multiple TSC13 homologs from GenBank were identified and three enzymes were chosen on the basis of homology levels. As illustrated in FIG. 3, SaTSC13 (Saccharomyces arboricola, GenBank accession No. EJS44333.1, amino acid SEQ ID NO: 3, DNA SEQ ID NO: 4) was the enzyme exhibiting highest homology (96%); SpTSC13 (Saccharomyces paradoxus, GenBank accession No. XP_0337652433.1, amino acid SEQ ID NO: 1, DNA SEQ ID NO: 2) was characterized by an intermediate level of homology (88%), while KbTSC13 (Kazachstania barnettii, GenBank accession No. 041405222.1, amino acid SEQ ID NO: 5, DNA SEQ ID NO: 6) was chosen as the enzyme bearing the lowest degree of homology (60%).

Resveratrol-producing S. cerevisiae strains featuring wild-type TSC13 (ScTSC13, amin acid SEQ ID NO: 9, DNA SEQ ID NO: 10) were transformed to replace the native ScTSC13 with each of three TSC13 homologs as described above. KbTSC13, SaTSC13 and SpTSC13 genes were synthesized (Twist BioScience, California), amplified and cloned into an integration vector under the control of the ScTSC13 native promoter using Gibson assembly cloning system (New England Biolabs, Massachusetts). The integration vector was digested using restriction enzymes to remove vector sequence before transformation. A number of colonies were picked from the transformation plate and inoculated on a 96-well microculture plate. After 48 hours of incubation at 30° C. to make the culture reach saturation, 80 μl of seed culture were inoculated into 48-well plates containing 1 ml of fermentation medium. The medium was composed of synthetic drop out medium without uracil buffered by 50 mM succinate (pH 6.0) with the addition of 40 g/L EnPump (Enpresso GmbH, Berlin, Germany), 0.4% reagent A, 2% vitamin solution (50 mg biotin, 200 mg p-aminobenzoic acid, 1 g nicotinic acid, 1 g Ca-pantothenate, 1 g pyridoxine-HCl, 1 g thiamine-HCl, 25 g myo-inositol per liter). The transformants were cultured in shaking incubator at 250 rpm at a temperature of 30° C., for a duration of 4 days.

Resveratrol and dihydroresveratrol were extracted by adding equal volumes of methanol. The samples were analyzed by high performance liquid chromatography (HPLC) using an Ultra C18 column (100×4.6 mm, packed with 3 μm particles). The chromatography was carried out using a Thermo Scientific Vanquish system. Mobile phase A was 50 ppm trifluoroacetic acid in water and mobile phase B was 50 ppm trifluoroacetic acid in 100% methanol. The chromatography was performed according to a linear gradient method with a 1.3 ml/minute flow rate, i.e., initial equilibration was 30% for B, linear gradient for 0 to 6.5 minutes 30% to 75% of B, then the percentage of B was kept stationary for two minutes, and the column was primed with 30% of B for 1 minute. Eluted compounds were detected by diode array illumination at the UV wavelength of 225 nm.

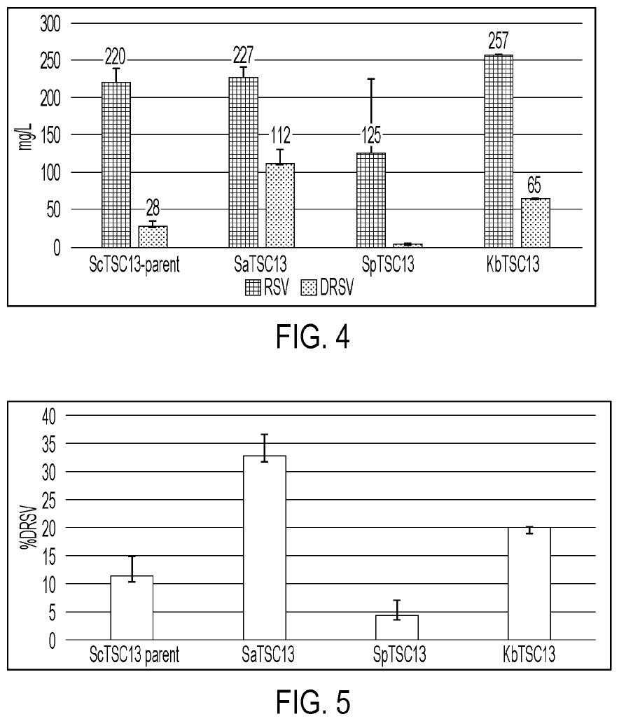

FIG. 4 shows the effect of native TSC13 replacement on dihydroresveratrol production in strain CNFS273. While resveratrol production was not changed substantially through various strains, dihydroresveratrol production was dramatically influenced depending on the transgenic TSC13 that was expressed in the strain. Compared to wild-type TSC13, SaTSC13 increased dihydroresveratrol production by a factor of four. KbTSC13 also increased dihdyroresveratrol by approximately two-fold. However, SpTSC13 decreased production of both dihydroresveratrol and resveratrol. As illustrated in FIG. 5, the share of dihydroresveratrol out of overall stilbenoid production was higher in the strains expressing SaTSC13 and KbTSC13 while the relative amount of dihydroresveratrol was reduced by half in the strain expressing SpTSC13.

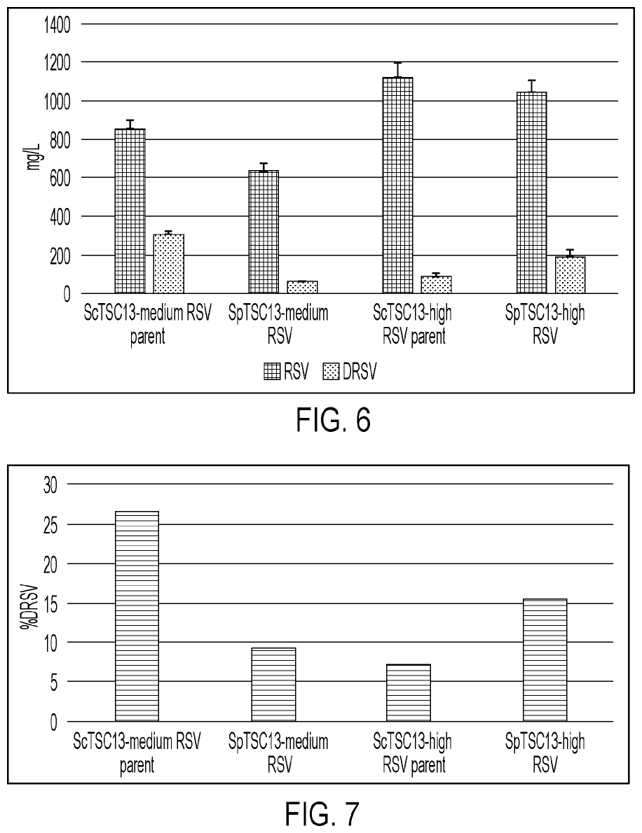

Another experiment was conducted to assess changes in the impact of SpTSC13 on dihydroresveratrol production depending on different strain backgrounds. Two S. cerevisiae strains were chosen: one was characterized by high (CNFS280), the other by intermediate (CNFS273) resveratrol production levels. Both strains were transformed with an SpTSC13 integration cassette. The experiment was conducted using the same procedure as set out above. As illustrated in FIGS. 6 and 7, dihydroresveratrol production consistently decreased in the strain having intermediate resveratrol production levels. However, dihydroresveratrol production increased two-fold by expressing SpTSC13 in the high resveratrol production strain.

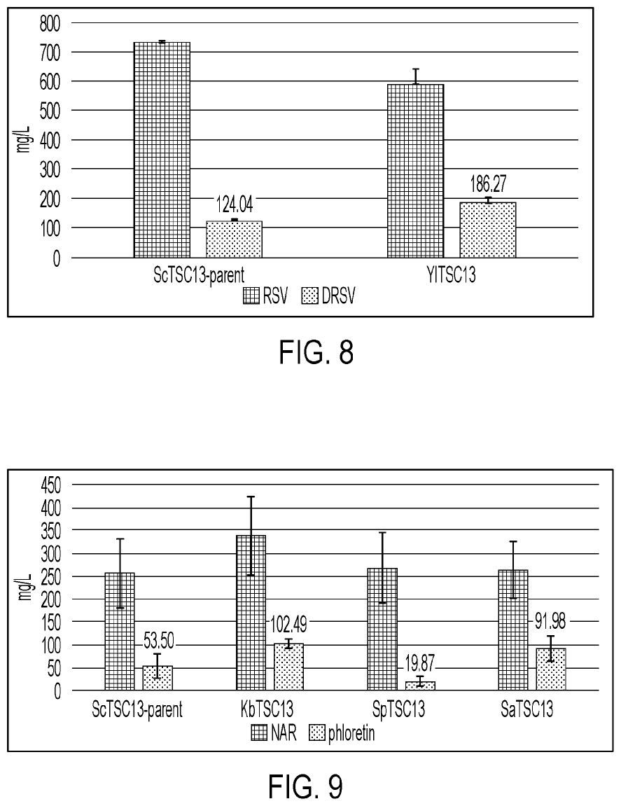

A resveratrol-producing S. cerevisiae strain comprising wild-type ScTSC13 was also transformed to replace the native TSC13 of S. cerevisiae with TSC13 from Yarrowia lipolytica (Y1TSC13, amino acid SEQ ID NO: 7, DNA SEQ ID NO: 8). Because the homology between Y1TSC13 and ScTSC13 was only 38%, Y1TSC13 was expressed under the strong constitutive TDH3 (SEQ ID NO: 28) promoter to compensate for its possible low activity. All other experimental procedures were the same as above. An Y1TSC13 integration cassette was transformed into a high resveratrol production strain. All other experimental procedures are the same as above. As illustrated in FIG. 8, Y1TSC13 also increased dihydroresveratrol production by about 1.5-fold while resveratrol production was slightly decreased.

### Example 3. Testing TSC13 Homologs in Naringenin-Producing Strains

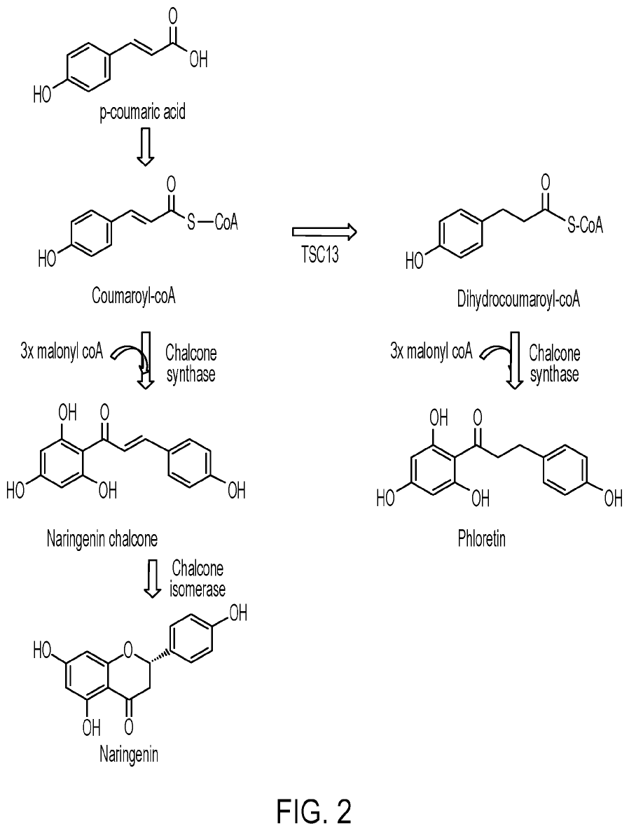

The three TSC13 homologs as set out above were tested in naringenin-producing strains to examine if the enzyme increases phloretin production. As illustrated in FIG. 2, phloretin is a side product of the naringenin pathway derived from dihydrocoumarolyl-CoA. A naringenin-producing S. cerevisiae strain was transformed with the above-characterized TSC13 homologue integration cassettes. All other experimental procedures are the same as above except the analytical steps, which were instead conducted as set out below. Naringenin was extracted by adding equal volumes of methanol. The samples were analyzed by high performance liquid chromatography (HPLC) using an Avantor ACE Excel 2 C18-PFP column (150×2.1 mm). The chromatography was operated using a Thermo Scientific Vanquish system. Mobile phase A was 0.1% trifluoroacetic acid in water and mobile phase B was 100% acetonitrile. The chromatography was performed by applying a linear gradient method with a 0.3 ml/minute flow rate, i.e., 0 minutes to 2 minutes 10% for B, linear gradient for 2 to 7 minutes 10% to 60% of B, then constant percentage of B for three minutes, and priming the column by 10% of B for 1 minute. Eluted compounds were detected by diode array illumination at the UV wavelength of 280 nm.

The results as reported in FIG. 8 show that the replacement of ScTSC13 with SaTSC13 or KbTSC13 increased phloretin production by two-fold. In contrast SpTSC13 decreased phloretin production by approximately two times. Naringenin production of all strains were similar except the strain expressing KbTSC13 whose naringenin production was slightly increased.

In view of the above, it will be seen that the several advantages of the disclosure are achieved, and other advantageous results attained. As various changes could be made in the above methods and systems without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

### Sequences

Amino acids
TSC13 homolog from Saccharomyces paradoxus
SEQ ID NO: 1
MPVTIKSRSKALRDTEIDLSKKPTLDDVLKQISTNNRNISKYRIRLTYKKETKQVPVISE
SFFREEADDSMEFFIKDLGPQISWRLVFFCEYLGPVLVHSLFYYLSTIPTVVDKWHSSSS
EYNPFLNKVAYFLILGHYGKRLFETLFVHQFSLATMPIFNLFKNCFHYWVLSGLISFGY
FGYGFPFGNAKLFKYYSYLKLDDLSTLIGLFVLSELWNFYCHIKLRLWGDYQKKHGN
AKVRVPLNQGIFNLFVAPNYTFEVWSWIWFTFVFKFNLFAVLFLTVSTVQMYAWAQK
KNKKYHTRRAFLIPFVF
Synthetic DNA
TSC13 homolog from Saccharomyces paradoxus
SEQ ID NO: 2
TATTTTGAATTTAATTTGAAAATGCCAGTTACTATCAAAAGCAGATCTAAGGCGTT
AAGAGATACTGAGATCGATTTGTCAAAGAAGCCCACATTGGACGATGTACTGAAA
CAGATATCCACCAACAACCGTAACATTTCTAAGTATCGTATAAGGTTGACATATAA
GAAGGAGACCAAGCAGGTTCCTGTTATTTCAGAATCTTTCTTCAGAGAGGAAGCTG
ATGATTCTATGGAATTCTTCATTAAGGATCTAGGACCCCAAATATCTTGGAGATTA
GTCTTCTTTTGCGAATATCTAGGTCCTGTCTTAGTGCATTCACTATTTTACTACCTA
AGTACTATTCCCACTGTGGTTGACAAATGGCATTCTTCTAGCAGTGAATACAACCC
ATTCTTAAATAAAGTAGCTTACTTTCTGATACTTGGTCACTACGGAAAGAGGCTAT
TCGAAACCCTGTTCGTTCACCAGTTTTCCTTAGCCACTATGCCAATATTCAATTTAT
TTAAGAATTGTTTCCACTACTGGGTTCTATCTGGTCTTATTTCGTTCGGCTACTTTG
GATACGGGTTTCCTTTCGGTAATGCTAAATTGTTCAAATACTATTCATACCTGAAA
TTGGACGACCTTTCCACACTGATAGGTCTTTTCGTGTTGTCTGAATTGTGGAATTTC
TACTGTCATATCAAATTACGTTTATGGGGTGACTACCAGAAGAAACACGGGAATG
CAAAAGTTAGAGTGCCTCTGAACCAAGGCATCTTCAATTTATTTGTCGCTCCTAAT
TACACTTTCGAGGTATGGTCTTGGATTTGGTTTACATTCGTATTTAAGTTTAATCTG
TTCGCTGTGTTGTTTCTGACAGTATCAACGGTACAGATGTATGCTTGGGCTCAGAA
GAAGAACAAGAAATACCATACCAGGAGAGCATTCTTGATCCCATTCGTCTTCTAG
Amino acids
TSC13 homolog from Saccharomyces aroboricola
SEQ ID NO: 3
MTVTIKSRSKGLRDTEVDLSGKPTLDDVLKQISSNNRNISKYRIRLTYKKEDKQVPVIS
DTFFQEKADDSMEFFIKDLGPQISWRLVFFCEYLGPILVHSLFYYLSTIPKIVEKCHSSSS
EYNPFLNRVGYSLIVAHYGKRLFETLFIHQFSLATMPIFNLFKNCFHYWVLSGLISFGYF
GYGFPFGNAKLFRYYSYLKLDDLGTLIGLFVVSELWNLYCHIKLRLWGDYQKKHGNS
KVRVPLNQGIFKLFVAPNYTFEVWSWVWFTFVFKLNLFAVLFLTVSAVQMYAWAQK
KNKKYHTRRAFLIPYIF
Synthetic DNA
TSC13 homolog from Saccharomyces aroboricola
SEQ ID NO: 4
ATGACAGTTACGATAAAGAGTCGTAGCAAGGGACTTAGGGATACCGAAGTGGATT
TATCTGGTAAGCCGACGCTTGACGACGTGTTAAAGCAAATATCATCAAATAATCGT
AATATATCCAAATACAGAATTAGGTTGACATATAAGAAGGAGGATAAACAGGTCC
CGGTCATTTCTGACACGTTCTTCCAAGAGAAGGCAGATGATTCTATGGAGTTCTTC
ATCAAAGACCTGGGACCCCAAATATCATGGAGACTGGTCTTCTTTTGTGAATACTT
GGGACCCATATTGGTGCATTCCCTTTTTTATTACCTAAGCACAATACCGAAGATTG
TCGAGAAATGCCATTCATCTTCTAGCGAATACAATCCCTTCCTAAATAGGGTTGGA
TATAGTCTTATCGTGGCGCATTACGGGAAAAGGCTGTTCGAAACTCTTTTCATCCA
CCAATTTAGCCTGGCAACTATGCCTATATTCAACCTATTCAAAAATTGCTTCCACT
ACTGGGTACTGAGTGGTCTGATTTCCTTTGGGTATTTTGGCTACGGCTTCCCTTTTG
GGAATGCGAAACTTTTCAGATACTACAGCTACTTAAAACTAGATGACTTAGGTACA
CTAATAGGTTTGTTCGTAGTCTCTGAATTATGGAATCTTTATTGTCACATAAAATTA
CGTTTGTGGGGTGATTATCAGAAGAAACACGGTAACTCAAAGGTCCGTGTGCCGTT
GAATCAAGGAATCTTCAAGCTTTTCGTGGCTCCTAACTACACGTTTGAAGTCTGGT
CATGGGTGTGGTTCACTTTTGTTTTTAAGCTAAATTTATTTGCGGTCCTGTTCCTTA
CTGTATCCGCAGTACAGATGTATGCGTGGGCTCAAAAGAAAAACAAAAAGTATCA
TACCAGACGTGCCTTTTTGATTCCCTACATCTTTTAA
Amino acids
TSC13 homolog from Kazachstania barnettii
SEQ ID NO: 5
MVAVKSRSKSLRDTEIAINKNSTLESVLIAISKNNKGISKYRLRVTYLKESKQVPITEES
FFNEGNNKDIELFVKDLGPQISWRLVFVIEYLGPLLVHSLVYYLSLNPKFMAKFGTSKI
NNVDPALNRIAFFMVMGHYLKREIETLFVHKFTLATMPLFNVFKNSFHYWILNGIIAL
GYLGYGFVVPNCCYEKTLNYVGLNNLNTLVSLFVLSEAWNAYIHIRLRIFGDQQRKIG
NNTKRVPISTGIFKIFVAPNYTFEVWSWIWFALAFKLNLFAVFFVTVSATQMYLWAM
KKNRRYGTKRSFIIPFIF
Synthetic DNA
TSC13 homolog from Kazachstania barnettii
SEQ ID NO: 6
ATGGTTGCAGTAAAGTCTAGAAGTAAGTCTTTAAGAGATACCGAAATTGCCATAA
ATAAGAATTCTACTCTTGAAAGTGTGTTGATCGCAATTTCAAAGAACAATAAAGGT
ATTTCAAAGTACAGATTGAGAGTCACATATCTGAAGGAGTCTAAACAAGTTCCAA
TAACTGAAGAAAGTTTCTTCAATGAAGGGAATAACAAGGATATCGAACTCTTCGTT
AAAGATTTAGGTCCGCAAATATCGTGGAGACTTGTGTTTGTTATTGAATATCTCGG
TCCTTTATTGGTACATTCTCTAGTTTATTATCTGAGTTTAAACCCAAAGTTTATGGC
TAAATTTGGCACAAGTAAGATTAATAATGTAGATCCTGCATTGAATAGAATCGCCT
TCTTCATGGTAATGGGCCACTATTTAAAGAGAGAAATCGAAACTTTGTTCGTTCAT
AAATTTACTCTTGCTACAATGCCATTATTTAATGTGTTTAAGAATAGCTTTCATTAC
TGGATCTTAAATGGCATCATTGCATTGGGTTATCTGGGATATGGGTTCGTGGTTCC
CAATTGTTGTTATGAGAAGACTTTGAACTATGTAGGCCTTAATAATTTGAATACCT
TAGTTTCGTTGTTTGTATTGTCTGAGGCTTGGAATGCGTATATCCACATCCGATTGC
GGATCTTCGGTGATCAACAAAGAAAGATAGGGAATAATACGAAGAGAGTTCCAAT
TTCGACAGGAATCTTCAAGATCTTCGTTGCTCCCAATTATACGTTTGAAGTTTGGTC
TTGGATTTGGTTCGCTTTGGCCTTCAAGCTCAACCTATTCGCAGTATTCTTCGTGAC
TGTGTCCGCTACACAAATGTACCTATGGGCCATGAAGAAGAACCGTAGATATGGT
ACTAAGCGTAGCTTTATTATACCCTTTATATTCTAA
Amino acids
TSC13 homolog from Yarrowia lipolitica
SEQ ID NO: 7
MVNLSLRPRPAKAKFKGLPANLEVSPEDTVASVVAKLSAATKLSKSRIRLTVADEENG
GAPGAKKKHIVLKPEHAVGDYLFSDSPVVFVKDLGPQIPWRTVFILEYLGPLLAHPIIFF
GQKFFYRQSFEYTFAQKLVFTLCMLHFLKREIETIYIHKFSSATMPLFNLFKNSGYYWF
IAGFNLAFFVYAPASFSSPQAPLWKRFLFSTGFFERTPLFLNLMATLWLWGETSNFWT
HFNLASLRNDGSKDHKIPFGYGFNLVSCPNYFFEVVSWIAIALMCGNWSAYVFTAIGF
GQMYVWAVQKHRRYKREFGDRYPRNRKVMVPFLL
Synthetic DNA
TSC13 homolog from Yarrowia lipolitica
SEQ ID NO: 8.
ATGGTCAATCTGTCTCTGCGTCCCCGTCCTGCCAAGGCCAAGTTCAAGGGTCTGCC
CGCCAACCTCGAGGTGTCTCCTGAGGACACGGTGGCCTCTGTGGTGGCGAAATTG
AGCGCTGCCACCAAACTGTCCAAGTCCCGAATTCGGCTCACTGTGGCCGATGAGG
AGAATGGAGGCGCTCCCGGAGCCAAGAAGAAGCACATTGTGCTCAAGCCCGAGC
ACGCTGTCGGCGATTACCTCTTCTCCGACTCTCCTGTGGTGTTTGTGAAGGACTTGG
GCCCCCAGATCCCCTGGAGAACCGTCTTCATTCTCGAGTACCTGGGACCTCTTCTG
GCCCACCCCATCATCTTCTTTGGCCAGAAGTTCTTTTACCGACAGAGCTTTGAGTA
CACGTTTGCTCAGAAGCTGGTCTTCACTCTATGCATGCTGCATTTCCTCAAGCGAG
AGATTGAGACCATCTACATCCACAAGTTCTCCTCCGCCACCATGCCTCTGTTCAAC
CTCTTTAAGAACTCCGGCTACTACTGGTTCATTGCCGGTTTCAACCTTGCCTTTTTC
GTTTATGCCCCCGCCTCCTTTTCTTCTCCCCAGGCTCCTCTGTGGAAGCGTTTCCTC
TTTTCCACTGGCTTCTTTGAGCGAACCCCTCTGTTCCTCAACCTCATGGCCACCCTG
TGGCTCTGGGGAGAGACCTCCAACTTCTGGACCCACTTCAACTTGGCTTCTCTGAG
AAACGATGGCTCCAAGGACCACAAGATCCCCTTTGGCTACGGCTTCAACCTTGTTT
CCTGCCCCAACTACTTCTTTGAGGTTGTCAGCTGGATCGCAATTGCCCTCATGTGT
GGCAACTGGTCCGCTTATGTCTTTACTGCCATTGGCTTCGGTCAGATGTACGTCTG
GGCCGTCCAGAAGCACCGACGATACAAGCGGGAGTTTGGAGACCGGTACCCTCGA
AACAGAAAGGTCATGGTTCCTTTCTTGCTTTAG
Amino acids
TSC13 from Saccharomyces cerevisiae
SEQ ID NO: 9
MPITIKSRSKGLRDTEIDLSKKPTLDDVLKKISANNHNISKYRIRLTYKKESKQVPVISES
FFQEEADDSMEFFIKDLGPQISWRLVFFCEYLGPVLVHSLFYYLSTIPTVVDRWHSASS
DYNPFLNRVAYFLILGHYGKRLFETLFVHQFSLATMPIFNLFKNCFHYWVLSGLISFGY
FGYGFPFGNAKLFKYYSYLKLDDLSTLIGLFVLSELWNFYCHIKLRLWGDYQKKHGN
AKIRVPLNQGIFNLFVAPNYTFEVWSWIWFTFVFKFNLFAVLFLTVSTAQMYAWAQK
KNKKYHTRRAFLIPFVF
Synthetic DNA
TSC13 from Saccharomyces cerevisiae
SEQ ID NO: 10
ATGCCTATCACCATAAAAAGCCGCTCTAAAGGGTTAAGGGACACTGAAATTGACT
TATCCAAAAAGCCTACTTTAGATGATGTTTTGAAAAAAATCTCTGCTAATAACCAC
AATATCAGCAAGTACAGGATAAGATTAACCTACAAAAAGGAATCTAAACAAGTTC
CGGTTATTTCAGAATCGTTTTTTCAAGAAGAGGCTGATGACTCAATGGAATTCTTC
ATCAAAGATTTGGGTCCCCAAATTTCATGGAGATTAGTCTTCTTTTGTGAGTATTTG
GGTCCAGTCTTGGTTCACTCCCTTTTTTATTATCTATCTACCATTCCCACAGTTGTTG
ATAGATGGCACAGTGCTAGCTCCGACTATAATCCATTTTTAAACAGGGTTGCATAT
TTTTTAATTTTAGGACATTATGGAAAGAGATTATTTGAAACCTTATTTGTTCACCAA
TTCTCTTTAGCTACTATGCCAATTTTCAACCTGTTCAAAAATTGTTTCCATTACTGG
GTTCTAAGCGGTCTCATTTCATTCGGTTACTTTGGCTACGGCTTCCCCTTTGGGAAT
GCTAAGTTATTCAAATACTATTCATATTTGAAATTGGATGACTTGAGTACATTAAT
TGGTCTTTTCGTGCTTTCAGAACTATGGAACTTTTATTGCCACATTAAATTGCGCCT
ATGGGGTGACTATCAAAAGAAGCATGGTAACGCTAAGATCCGTGTCCCATTGAAT
CAAGGTATTTTCAATCTTTTTGTTGCTCCCAACTATACTTTTGAAGTTTGGTCTTGG
ATTTGGTTTACTTTTGTGTTCAAGTTCAATTTATTTGCCGTTTTATTTTTGACTGTTT
CAACAGCTCAAATGTACGCATGGGCTCAAAAGAAAAACAAAAAGTATCATACCAG
AAGAGCATTCTTGATTCCATTTGTATTTTGA
Amino acid
Arabidopsis thaliana coumaroyl CoA ligase 1
SEQ ID NO: 11
MAPQEQAVSQVMEKQSNNNNSDVIFRSKLPDIYIPNHLSLHDYIFQNISEFATKPCLING
PTGHVYTYSDVHVISRQIAANFHKLGVNQNDVVMLLLPNCPEFVLSFLAASFRGATAT
AANPFFTPAEIAKQAKASNTKLIITEARYVDKIKPLQNDDGVVIVCIDDNESVPIPEGCL
RFTELTQSTTEASEVIDSVEISPDDVVALPYSSGTTGLPKGVMLTHKGLVTSVAQQVD
GENPNLYFHSDDVILCVLPMFHIYALNSIMLCGLRVGAAILIMPKFEINLLWELIQRCK
VTVAPMVPPIVLAIAKSSETEKYDLSSIRVVKSGAAPLGKELEDAVNAKFPNAKLGQG
YGMTEAGPVLAMSLGFAKEPFPVKSGACGTVVRNAEMKIVDPDTGDSLSRNQPGEICI
RGHQIMKGYLNNPAATAETIDKDGWLHTGDIGLIDDDDELFIVDRLKELIKYKGFQVA
PAELEALLIGHPDITDVAVVAMKEEAAGEVPVAFVVKSKDSELSEDDVKQFVSKQVV
FYKRINKVFFTESIPKAPSGKILRKDLRAKLANGL
Synthetic DNA
Codon optimized Arabidopsis thaliana coumaroyl CoA ligase 1
SEQ ID NO: 12
ATGGCGCCACAAGAACAAGCAGTTTCTCAGGTGATGGAGAAACAGAGCAACAAC
AACAACAGTGACGTCATTTTCCGATCAAAGTTACCGGATATTTACATCCCGAACCA
CCTATCTCTCCACGACTACATCTTCCAAAACATCTCCGAATTCGCCACTAAGCCTT
GCCTAATCAACGGACCAACCGGCCACGTGTACACTTACTCCGACGTCCACGTCATC
TCCCGCCAAATCGCCGCCAATTTTCACAAACTCGGCGTTAACCAAAACGACGTCGT
CATGCTCCTCCTCCCAAACTGTCCCGAATTCGTCCTCTCTTTCCTCGCCGCCTCCTT
CCGCGGCGCAACCGCCACCGCCGCAAACCCTTTCTTCACTCCGGCGGAGATAGCT
AAACAAGCCAAAGCCTCCAACACCAAACTCATAATCACCGAAGCTCGTTACGTCG
ACAAAATCAAACCACTTCAAAACGACGACGGAGTAGTCATCGTCTGCATCGACGA
CAACGAATCCGTGCCAATCCCTGAAGGCTGCCTCCGCTTCACCGAGTTGACTCAGT
CGACAACCGAGGCATCAGAAGTCATCGACTCGGTGGAGATTTCACCGGACGACGT
GGTGGCACTACCTTACTCCTCTGGCACGACGGGATTACCAAAAGGAGTGATGCTG
ACTCACAAGGGACTAGTCACGAGCGTTGCTCAGCAAGTCGACGGCGAGAACCCGA
ATCTTTATTTCCACAGCGATGACGTCATACTCTGTGTTTTGCCCATGTTTCATATCT
ACGCTTTGAACTCGATCATGTTGTGTGGTCTTAGAGTTGGTGCGGCGATTCTGATA
ATGCCGAAGTTTGAGATCAATCTGCTATGGGAGCTGATCCAGAGGTGTAAAGTGA
CGGTGGCTCCGATGGTTCCGCCGATTGTGTTGGCCATTGCGAAGTCTTCGGAAACG
GAGAAGTATGATTTGAGCTCGATAAGAGTGGTGAAATCTGGTGCTGCTCCTCTTGG
TAAAGAACTTGAAGATGCCGTTAATGCCAAGTTTCCTAATGCCAAACTCGGTCAGG
GATACGGAATGACGGAAGCAGGTCCAGTGCTAGCAATGTCGTTAGGTTTTGCAAA
GGAACCTTTTCCGGTTAAGTCAGGAGCTTGTGGTACTGTTGTAAGAAATGCTGAGA
TGAAAATAGTTGATCCAGACACCGGAGATTCTCTTTCGAGGAATCAACCCGGTGA
GATTTGTATTCGTGGTCACCAGATCATGAAAGGTTACCTCAACAATCCGGCAGCTA
CAGCAGAAACCATTGATAAAGACGGTTGGCTTCATACTGGAGATATTGGATTGAT
CGATGACGATGACGAGCTTTTCATCGTTGATCGATTGAAAGAACTTATCAAGTATA
AAGGTTTTCAGGTAGCTCCGGCTGAGCTAGAGGCTTTGCTCATCGGTCATCCTGAC
ATTACTGATGTTGCTGTTGTCGCAATGAAAGAAGAAGCAGCTGGTGAAGTTCCTGT
TGCATTTGTGGTGAAATCGAAGGATTCGGAGTTATCAGAAGATGATGTGAAGCAA
TTCGTGTCGAAACAGGTTGTGTTTTACAAGAGAATCAACAAAGTGTTCTTCACTGA
ATCCATTCCTAAAGCTCCATCAGGGAAGATATTGAGGAAAGATCTGAGGGCAAAA
CTAGCAAATGGATTGTGA
Amino acid
Arabidopsis thaliana coumaroyl CoA ligase 2
SEQ ID NO: 13
MTTQDVIVNDQNDQKQCSNDVIFRSRLPDIYIPNHLPLHDYIFENISEFAAKPCLINGPT
GEVYTYADVHVTSRKLAAGLHNLGVKQHDVVMILLPNSPEVVLTFLAASFIGAITTSA
NPFFTPAEISKQAKASAAKLIVTQSRYVDKIKNLQNDGVLIVTTDSDAIPENCLRFSELT
QSEEPRVDSIPEKISPEDVVALPFSSGTTGLPKGVMLTHKGLVTSVAQQVDGENPNLYF
NRDDVILCVLPMFHIYALNSIMLCSLRVGATILIMPKFEITLLLEQIQRCKVTVAMVVPP
IVLAIAKSPETEKYDLSSVRMVKSGAAPLGKELEDAISAKFPNAKLGQGYGMTEAGPV
LAMSLGFAKEPFPVKSGACGTVVRNAEMKILDPDTGDSLPRNKPGEICIRGNQIMKGY
LNDPLATASTIDKDGWLHTGDVGFIDDDDELFIVDRLKELIKYKGFQVAPAELESLLIG
HPEINDVAVVAMKEEDAGEVPVAFVVRSKDSNISEDEIKQFVSKQVVFYKRINKVFFT
DSIPKAPSGKILRKDLRARLANGLMN
Synthetic DNA
Codon optimized Arabidopsis thaliana coumaroyl CoA ligase 2
SEQ ID NO: 14
ATGACTACGCAGGATGTTATTGTCAATGATCAAAATGACCAAAAGCAATGTTCGA
ATGATGTTATCTTTCGTAGTAGACTCCCTGATATATACATACCTAACCATCTACCAT
TGCATGATTACATATTTGAAAATATATCGGAATTTGCTGCTAAGCCATGCCTAATC
AATGGTCCAACAGGTGAAGTGTATACCTATGCTGATGTTCATGTTACTTCCAGGAA
GCTCGCTGCTGGTTTGCACAACTTGGGCGTTAAACAGCATGACGTCGTTATGATAT
TGCTGCCAAATAGCCCAGAAGTGGTACTTACTTTCTTGGCCGCCTCGTTTATTGGC
GCCATTACGACATCCGCAAATCCCTTCTTCACGCCCGCTGAAATTTCTAAACAAGC
TAAAGCATCTGCTGCTAAATTAATCGTCACACAAAGTAGATATGTTGATAAGATTA
AGAACTTACAAAACGATGGGGTCTTAATTGTCACAACCGATTCTGATGCTATCCCT
GAAAATTGTCTGAGATTCTCTGAGTTAACTCAATCCGAAGAGCCTAGAGTAGACA
GTATACCTGAGAAGATCTCTCCAGAAGATGTGGTGGCTTTGCCATTTTCCTCAGGT
ACTACCGGTCTGCCAAAGGGTGTGATGTTGACTCACAAGGGTTTGGTGACGTCAGT
AGCTCAGCAAGTAGATGGGGAGAACCCTAATCTGTATTTCAATAGAGATGACGTC
ATTTTGTGCGTATTACCTATGTTCCATATTTATGCATTAAACTCGATTATGCTATGC
TCTCTGCGAGTTGGAGCAACTATATTAATCATGCCAAAGTTTGAGATAACTCTCTT
GTTAGAACAAATTCAGAGGTGCAAGGTCACTGTTGCTATGGTAGTACCACCAATA
GTCCTGGCAATCGCAAAGAGTCCTGAAACCGAGAAGTATGATTTAAGTAGTGTGC
GGATGGTTAAATCAGGCGCTGCCCCTCTAGGTAAAGAATTAGAAGATGCCATTTCC
GCTAAATTTCCGAATGCAAAATTAGGCCAAGGATATGGCATGACGGAAGCTGGTC
CAGTTCTAGCAATGTCTTTGGGGTTTGCTAAAGAGCCTTTTCCCGTAAAGAGCGGT
GCCTGTGGCACTGTTGTGCGTAATGCTGAGATGAAAATACTGGATCCAGACACGG
GCGATTCACTACCACGCAATAAACCAGGCGAGATATGTATAAGGGGAAACCAGAT
TATGAAGGGGTATTTGAACGATCCCCTGGCCACCGCCTCAACTATCGATAAGGAC
GGATGGTTACACACTGGTGACGTTGGGTTTATTGACGATGATGATGAATTATTCAT
CGTTGACAGATTAAAGGAATTGATCAAATACAAAGGTTTTCAAGTAGCTCCAGCA
GAACTCGAAAGCCTTTTGATTGGACATCCAGAGATAAATGACGTCGCAGTGGTCG
CTATGAAAGAAGAGGATGCTGGTGAAGTTCCCGTTGCATTTGTAGTTAGATCGAA
GGATTCCAACATTAGCGAGGACGAAATTAAACAATTTGTAAGCAAACAGGTTGTC
TTTTATAAAAGAATCAATAAAGTTTTCTTCACTGACTCAATTCCAAAGGCCCCTTCT
GGTAAAATCCTGCGTAAGGACTTGAGGGCACGATTGGCTAATGGCCTCATGAATT
GA
Amino acids
Ammonia phenylalanine lyase from Arabidopsis thaliana
SEQ ID NO: 15
MDQIEAMLCGGGEKTKVAVTTKTLADPLNWGLAADQMKGSHLDEVKKMVEEYRRP
VVNLGGETLTIGQVAAISTVGGSVKVELAETSRAGVKASSDWVMESMNKGTDSYGV
TTGFGATSHRRTKNGTALQTELIRFLNAGIFGNTKETCHTLPQSATRAAMLVRVNTLL
QGYSGIRFEILEAITSLLNHNISPSLPLRGTITASGDLVPLSYIAGLLTGRPNSKATGPDG
ESLTAKEAFEKAGISTGFFDLQPKEGLALVNGTAVGSGMASMVLFEANVQAVLAEVL
SAIFAEVMSGKPEFTDHLTHRLKHHPGQIEAAAIMEHILDGSSYMKLAQKVHEMDPLQ
KPKQDRYALRTSPQWLGPQIEVIRQATKSIEREINSVNDNPLIDVSRNKAIHGGNFQGT
PIGVSMDNTRLAIAAIGKLMFAQFSELVNDFYNNGLPSNLTASSNPSLDYGFKGAEIA
MASYCSELQYLANPVTSHVQSAEQHNQDVNSLGLISSRKTSEAVDILKLMSTTFLVGI
CQAVDLRHLEENLRQTVKNTVSQVAKKVLTTGINGELHPSRFCEKDLLKVVDREQVF
TYVDDPCSATYPLMQRLRQVIVDHALSNGETEKNAVTSIFQKIGAFEEELKAVLPKEV
EAARAAYGNGTAPIPNRIKECRSYPLYRFVREELGTKLLTGEKVVSPGEEFDKVFTAM
CEGKLIDPLMDCLKEWNGAPIPIC
Synthetic DNA
Codon optimized Arabidopsis thaliana phenylalanine ammonia lyase
SEQ ID NO: 16
CTAACAAATCGGTATTGGAGCACCGTTCCATTCCTTTAAGCAATCCATCAACGGGT
CTATTAGTTTACCCTCACACATTGCAGTAAAGACTTTATCAAATTCTTCCCCGGGA
GAAACTACCTTTTCACCTGTAAGTAACTTTGTGCCAAGCTCTTCTCTAACAAATCTG
TATAAGGGGTAGGATCTACACTCCTTTATACGATTAGGTATTGGGGCAGTTCCGTT
ACCATAGGCAGCTCTTGCAGCTTCCACCTCTTTTGGTAGTACGGCTTTTAGCTCTTC
TTCAAATGCACCAATTTTCTGGAAAATAGAAGTGACGGCATTCTTCTCGGTTTCTC
CATTTGATAAGGCATGGTCTACAATTACTTGACGTAATCTCTGCATTAAGGGATAC
GTAGCTGAACAGGGATCATCAACATAAGTGAAAACTTGTTCCCTGTCAACAACCTT
TAGCAAATCCTTTTCACAAAACCTGGAGGGGTGTAATTCGCCGTTAATTCCGGTTG
TTAAGACCTTCTTGGCAACCTGACTTACAGTATTCTTAACAGTCTGTCTCAGGTTCT
CTTCAAGATGTCTAAGGTCAACAGCCTGGCAGATTCCAACTAGAAATGTTGTAGAC
ATCAACTTGAGTATGTCTACTGCTTCGGAAGTCTTTCTCGATGAAATTAAACCGAG
AGAGTTCACGTCCTGATTATGTTGTTCTGCGGATTGCACATGAGATGTTACAGGAT
TGGCTAAATATTGGAGCTCCGAACAGTAACTTGCCATGGCTATTTCTGCTCCTTTG
AAACCGTAATCTAAGGATGGGTTACTGGAAGCGGTTAAATTGGACGGTAGTCCAT
TGTTATAGAAATCGTTCACCAATTCTGAAAATTGAGCGAACATTAATTTACCAATC
GCCGCGATAGCTAGTCTCGTGTTGTCCATTGACACACCAATAGGTGTCCCTTGGAA
GTTACCACCATGAATGGCTTTATTGCGGCTGACGTCAATTAGAGGATTATCATTCA
CGGAATTAATCTCCCTTTCGATGCTCTTAGTGGCTTGTCTTATTACTTCAATTTGTG
GGCCCAGCCATTGTGGTGAGGTTCTCAAAGCATACCTATCTTGTTTAGGCTTTTGT
AGTGGATCCATTTCATGCACCTTTTGAGCGAGTTTCATATAGGAGCTCCCATCCAA
AATGTGTTCCATTATAGCAGCTGCCTCAATTTGACCTGGATGATGCTTCAATCTAT
GAGTCAAATGATCAGTAAACTCTGGTTTTCCTGACATCACTTCCGCAAATATAGCC
GACAGCACCTCAGCCAACACTGCTTGAACATTGGCCTCGAAAAGAACCATACTGG
CCATACCCGAACCAACTGCTGTCCCATTAACAAGCGCTAGGCCTTCCTTGGGTTGA
AGATCAAAGAATCCAGTCGATATGCCTGCCTTTTCGAATGCCTCCTTTGCCGTAAG
AGATTCCCCATCTGGGCCGGTTGCTTTAGAATTAGGTCTACCTGTTAATAATCCTG
CAATATAAGACAGTGGTACAAGATCACCACTGGCCGTTATAGTGCCCCTTAATGGT
AGTGAAGGGCTGATATTATGGTTCAGAAGGGACGTTATAGCTTCAAGGATTTCGA
ACCTAATACCCGAATAACCCTGCAAAAGAGTATTTACCCTTACCAACATTGCAGCT
CTGGTAGCTGATTGAGGTAAAGTATGACATGTCTCTTTGGTATTGCCAAAGATTCC
AGCGTTCAAGAAGCGGATTAGTTCAGTTTGAAGAGCAGTACCATTCTTTGTCCTCC
TATGCGAGGTTGCCCCGAAACCGGTAGTCACACCGTATGAATCCGTGCCTTTGTTC
ATACTTTCCATTACCCAATCTGAAGAAGCTTTTACGCCAGCCCTAGATGTTTCCGC
TAATTCAACTTTGACGCTGCCACCAACGGTACTAATAGCCGCTACTTGTCCTATCG
TCAAAGTTTCACCTCCTAGATTCACCACGGGCCTACGGTACTCTTCGACCATTTTCT
TAACTTCATCAAGATGGCTTCCTTTCATCTGGTCTGCGGCCAGTCCCCAGTTTAAA
GGATCAGCCAATGTCTTAGTAGTAACGGCCACTTTAGTCTTTTCGCCACCTCCACA
GAGCATTGCTTCGATTTGATCCAT
Amino acids
Cinnamic acid 4-hydroxylase from Arabidopsis thaliana
SEQ ID NO: 17
MDLLLLEKSLIAVFVAVILATVISKLRGKKLKLPPGPIPIPIFGNWLQVGDDLNHRNLVD
YAKKFGDLFLLRMGQRNLVVVSSPDLTKEVLLTQGVEFGSRTRNVVFDIFTGKGQDM
VFTVYGEHWRKMRRIMTVPFFTNKVVQQNREGWEFEAASVVEDVKKNPDSATKGIV
LRKRLQLMMYNNMFRIMFDRRFESEDDPLFLRLKALNGERSRLAQSFEYNYGDFIPIL
RPFLRGYLKICQDVKDRRIALFKKYFVDERKQIASSKPTGSEGLKCAIDHILEAEQKGEI
NEDNVLYIVENINVAAIETTLWSIEWGIAELVNHPEIQSKLRNELDTVLGPGVQVTEPD
LHKLPYLQAVVKETLRLRMAIPLLVPHMNLHDAKLAGYDIPAESKILVNAWWLANNP
NSWKKPEEFRPERFFEEESHVEANGNDFRYVPFGVGRRSCPGIILALPILGITIGRMVQN
FELLPPPGQSKVDTSEKGGQFSLHILNHSIIVMKPRNC
Synthetic DNA
Codon optimized Cinnamic acid 4-hydroxylase from Arabidopsis thaliana
SEQ ID NO: 18
ATGGACTTGTTGTTGTTGGAAAAGTCTTTGATCGCTGTTTTCGTTGCTGTTATCTTG
GCTACTGTTATCTCTAAGTTGAGAGGTAAGAAGTTGAAGTTGCCACCAGGTCCAAT
CCCAATCCCAATCTTCGGTAACTGGTTGCAAGTTGGTGACGACTTGAACCACAGAA
ACTTGGTTGACTACGCTAAGAAGTTCGGTGACTTGTTCTTGTTGAGAATGGGTCAA
AGAAACTTGGTTGTTGTTTCTTCTCCAGACTTGACTAAGGAAGTTTTGTTGACTCAA
GGTGTTGAATTTGGTTCAAGAACTAGAAACGTTGTTTTCGACATCTTCACTGGTAA
GGGTCAAGACATGGTTTTCACTGTTTACGGTGAACACTGGAGAAAGATGAGAAGA
ATCATGACTGTTCCATTCTTCACTAACAAGGTTGTTCAACAAAACAGAGAAGGTTG
GGAATTTGAAGCTGCTTCTGTTGTTGAAGATGTTAAGAAGAACCCAGACTCTGCTA
CTAAGGGTATCGTTTTGAGAAAGAGATTGCAATTGATGATGTACAACAACATGTTC
AGAATCATGTTCGACAGAAGATTCGAATCTGAAGATGACCCATTGTTCTTGAGATT
GAAGGCTTTGAACGGTGAAAGATCAAGATTGGCTCAATCTTTCGAATACAACTAC
GGTGACTTCATCCCAATCTTAAGACCATTCTTGAGAGGTTACTTGAAGATCTGTCA
AGACGTTAAGGACAGAAGAATCGCTTTGTTCAAGAAGTACTTCGTTGACGAAAGA
AAGCAAATCGCTTCTTCTAAGCCAACTGGTTCTGAAGGTTTGAAGTGTGCTATCGA
CCACATCTTGGAAGCTGAACAAAAGGGTGAAATCAACGAAGATAACGTTTTGTAC
ATCGTTGAAAACATCAACGTTGCTGCTATCGAAACTACTTTGTGGTCTATCGAATG
GGGTATCGCTGAATTGGTTAACCACCCAGAAATCCAATCTAAGTTGAGAAACGAA
TTGGACACTGTTTTGGGTCCAGGTGTTCAAGTTACTGAACCAGACTTGCACAAGTT
GCCATACTTGCAAGCTGTTGTTAAGGAAACTTTGAGATTGAGAATGGCTATCCCAT
TGTTGGTTCCACACATGAACTTGCACGACGCTAAGTTGGCTGGTTACGACATCCCA
GCTGAATCTAAGATCTTGGTTAACGCTTGGTGGTTGGCTAACAACCCAAACTCTTG
GAAGAAGCCAGAAGAATTTAGACCAGAAAGATTCTTCGAAGAAGAATCTCACGTT
GAAGCTAACGGTAACGACTTCAGATACGTTCCATTCGGTGTTGGTAGAAGATCTTG
TCCAGGTATCATCTTGGCTTTGCCAATCTTGGGTATCACTATCGGTAGAATGGTTC
AAAACTTCGAATTGTTGCCACCACCAGGTCAATCTAAGGTTGACACTTCTGAAAAG
GGTGGTCAATTCTCTTTGCACATCTTGAACCACTCTATCATCGTTATGAAGCCAAG
AAACTGTTAA
Amino acid
Vitis vinifera stilbene synthase
SEQ ID NO: 19
MASVEEFRNAQRAKGPATILAIGTATPDHCVYQSDYADYYFKVTKSEHMTALKKKFN
RICDKSMIKKRYIHLTEEMLEEHPNIGAYMAPSLNIRQEIITAEVPKLGKEAALKALKE
WGQPKSKITHLVFCTTSGVEMPGADYKLANLLGLEPSVRRVMLYHQGCYAGGTVLR
TAKDLAENNAGARVLVVCSEITVVTFRGPSEDALDSLVGQALFGDGSAAVIVGSDPDI
SIERPLFQLVSAAQTFIPNSAGAIAGNLREVGLTFHLWPNVPTLISENVEKCLTQAFDPL
GISDWNSLFWIAHPGGPAILDAVEAKLNLDKKKLEATRHVLSEYGNMSSACVLFILDE
MRKKSLKGERATTGEGLDWGVLFGFGPGLTIETVVLHSIPMVTN
Synthetic DNA
Codon optimized Vitis vinifera stilbene synthase opt1
SEQ ID NO: 20
ATGGCTTCTGTTGAGGAATTTAGGAATGCTCAACGTGCCAAGGGACCCGCCACTAT
TCTGGCTATAGGTACTGCCACCCCAGATCATTGCGTATATCAATCGGATTACGCTG
ACTACTACTTCAAGGTTACCAAAAGTGAGCACATGACAGCCTTGAAGAAGAAGTT
TAACCGTATATGCGATAAGTCAATGATCAAGAAAAGATACATTCACTTGACAGAA
GAAATGTTAGAGGAACATCCAAATATAGGCGCTTACATGGCTCCATCGTTAAACA
TCCGTCAGGAAATCATTACAGCTGAAGTACCCAAATTAGGTAAAGAGGCTGCATT
GAAAGCCCTAAAAGAATGGGGCCAACCTAAATCCAAAATTACTCATTTGGTATTCT
GTACCACAAGCGGCGTTGAAATGCCTGGAGCTGACTATAAACTTGCCAACCTACT
GGGCTTGGAACCTTCCGTCCGTAGGGTAATGCTTTACCACCAAGGTTGTTATGCTG
GTGGGACAGTCTTGAGGACGGCTAAGGACTTAGCCGAAAATAATGCTGGGGCACG
GGTTCTAGTTGTATGTTCGGAAATTACGGTTGTAACTTTTCGTGGTCCATCAGAAG
ATGCATTAGATTCGTTGGTCGGTCAGGCATTATTTGGCGATGGCTCCGCAGCAGTC
ATCGTCGGTTCGGATCCAGATATTAGTATAGAGCGCCCCTTGTTCCAACTCGTATC
CGCAGCTCAAACATTTATTCCAAACTCCGCGGGTGCGATTGCCGGGAACTTACGG
GAAGTGGGTTTAACCTTTCACCTCTGGCCAAATGTTCCTACCCTTATTTCCGAAAA
CGTTGAGAAATGCCTAACACAAGCTTTCGATCCTCTAGGAATCTCGGATTGGAATA
GCTTGTTCTGGATTGCCCATCCAGGTGGTCCTGCCATTCTTGATGCGGTTGAGGCT
AAATTGAACCTAGACAAGAAGAAGTTGGAAGCCACAAGACATGTACTGTCAGAAT
ATGGAAATATGAGTTCTGCCTGTGTCTTATTCATACTCGACGAAATGAGAAAGAAG
TCCTTAAAGGGCGAAAGAGCTACTACCGGCGAAGGACTAGATTGGGGAGTTTTGT
TTGGTTTCGGTCCTGGATTGACAATTGAAACAGTTGTTTTGCATAGTATTCCCATGG
TTACCAATTAA
Synthetic DNA
Codon optimized Vitis vinifera stilbene synthase opt2
SEQ ID NO: 21
ATGGCTAGCGTGGAGGAATTTAGGAATGCACAGAGAGCGAAAGGGCCTGCTACCA
TTTTAGCAATCGGTACTGCGACTCCAGATCATTGTGTATACCAAAGTGATTATGCA
GACTATTATTTCAAGGTCACCAAGTCTGAACACATGACCGCATTAAAGAAGAAGT
TTAATAGAATATGCGATAAGAGCATGATCAAGAAACGTTATATTCACTTGACGGA
AGAAATGTTGGAAGAACATCCTAATATAGGTGCTTACATGGCACCCTCTTTGAATA
TCAGACAGGAAATAATTACGGCAGAAGTTCCCAAATTGGGAAAAGAGGCTGCCTT
GAAGGCTTTAAAAGAATGGGGTCAGCCCAAATCTAAAATTACCCACTTAGTATTTT
GTACGACATCAGGCGTCGAAATGCCAGGTGCGGATTACAAATTAGCCAATTTGTT
AGGTTTGGAACCGTCAGTTAGACGTGTTATGTTGTACCATCAAGGATGCTATGCCG
GTGGGACGGTTCTGAGAACAGCGAAAGATCTAGCTGAGAATAACGCAGGCGCAA
GAGTATTGGTAGTCTGTTCCGAAATAACTGTTGTCACTTTCAGAGGCCCAAGTGAG
GACGCGTTGGACTCATTAGTTGGTCAGGCACTGTTTGGCGATGGTTCTGCCGCTGT
AATTGTCGGTAGCGACCCTGATATAAGTATTGAAAGACCCCTGTTCCAATTGGTTT
CAGCAGCACAAACTTTTATTCCTAATAGTGCTGGTGCTATCGCTGGTAATTTAAGA
GAAGTTGGCTTAACATTTCATTTGTGGCCTAATGTTCCAACCCTGATAAGCGAAAA
CGTAGAGAAATGTCTTACCCAAGCGTTCGACCCATTAGGAATTAGTGATTGGAACT
CTCTTTTCTGGATCGCACACCCAGGAGGCCCAGCTATATTAGACGCAGTTGAAGCT
AAGTTAAATTTAGATAAGAAGAAATTGGAGGCAACAAGACATGTGTTATCCGAGT
ACGGAAATATGTCATCAGCATGTGTGTTGTTTATATTGGACGAGATGAGAAAGAA
GAGTCTTAAGGGAGAGAGAGCTACCACAGGAGAGGGATTGGATTGGGGTGTCTTA
TTTGGTTTTGGTCCAGGTCTAACAATTGAAACAGTAGTGTTACACTCTATTCCAAT
GGTCACAAATTAA
Synthetic DNA
Codon optimized Vitis vinifera stilbene synthase opt3
SEQ ID NO: 22
ATGGCATCCGTGGAAGAATTTAGAAACGCACAGAGGGCAAAAGGTCCAGCAACC
ATACTAGCTATCGGCACAGCTACCCCTGATCATTGCGTCTATCAGTCGGACTACGC
TGATTATTATTTTAAGGTTACCAAATCAGAACACATGACCGCATTGAAGAAGAAGT
TTAACAGAATATGTGACAAATCAATGATTAAGAAGCGCTATATTCATCTAACTGAG
GAGATGCTGGAGGAACATCCAAATATTGGTGCGTACATGGCACCATCCCTAAACA
TTCGCCAAGAGATTATTACGGCTGAAGTTCCCAAGTTAGGCAAGGAAGCAGCTCT
GAAGGCATTAAAGGAGTGGGGCCAGCCTAAGAGCAAAATCACTCATCTTGTATTT
TGTACGACCTCTGGTGTGGAAATGCCTGGAGCTGACTATAAATTAGCGAACTTGTT
GGGCCTAGAGCCAAGTGTTAGAAGGGTGATGCTGTATCATCAGGGTTGTTATGCA
GGTGGTACTGTCTTGAGGACAGCCAAGGATCTGGCTGAAAATAATGCTGGCGCCA
GAGTACTCGTAGTATGCAGTGAGATCACCGTCGTCACATTTAGGGGACCATCTGAA
GATGCTTTGGATTCTCTCGTTGGCCAGGCTTTATTCGGCGATGGTTCCGCTGCTGTG
ATAGTCGGCTCGGATCCTGACATATCCATCGAACGCCCCTTGTTTCAATTAGTTAG
CGCAGCGCAGACCTTTATACCTAACTCGGCCGGGGCAATAGCAGGTAATTTGCGT
GAAGTCGGATTGACTTTTCATTTGTGGCCTAACGTCCCCACGTTGATTTCAGAAAA
TGTCGAAAAGTGTTTAACGCAAGCATTCGATCCTCTAGGTATATCTGATTGGAATA
GCCTCTTCTGGATTGCACATCCTGGCGGGCCTGCTATTCTGGACGCGGTCGAGGCT
AAGTTAAATTTGGATAAGAAGAAGCTGGAAGCCACCAGACATGTCCTGTCTGAGT
ACGGGAATATGTCAAGTGCATGTGTGCTCTTTATACTGGACGAGATGAGGAAGAA
ATCGTTAAAGGGTGAGAGAGCTACTACGGGTGAAGGATTAGATTGGGGCGTATTA
TTCGGCTTCGGTCCGGGGCTCACTATCGAAACAGTAGTCCTGCATAGTATCCCCAT
GGTCACCAATTGA
Synthetic DNA
Codon optimized Vitis vinifera stilbene synthase opt4
SEQ ID NO: 23
ATGGCCTCAGTAGAAGAGTTTCGTAATGCTCAAAGAGCCAAGGGCCCAGCTACAA
TTTTAGCTATAGGCACCGCTACGCCAGATCATTGTGTTTACCAATCCGATTACGCA
GATTACTATTTCAAGGTCACAAAGAGCGAACACATGACTGCCTTAAAGAAGAAAT
TTAACCGTATCTGTGACAAATCTATGATCAAGAAGCGTTACATACATTTGACTGAA
GAGATGTTAGAGGAGCACCCTAACATTGGTGCCTACATGGCACCGTCGTTAAATAT
CCGTCAAGAAATTATTACAGCTGAGGTCCCAAAGTTAGGTAAGGAAGCTGCTCTT
AAAGCCTTGAAGGAATGGGGTCAACCTAAGAGTAAAATTACACATTTGGTCTTTTG
TACCACTTCCGGCGTTGAAATGCCTGGCGCCGATTACAAGTTAGCTAACCTATTAG
GTCTGGAACCAAGCGTTCGTCGCGTAATGTTATACCATCAGGGATGTTATGCAGGT
GGTACTGTATTAAGGACCGCAAAAGACTTGGCAGAAAATAACGCGGGCGCCAGA
GTATTGGTCGTGTGTAGCGAAATTACGGTTGTAACATTCAGGGGTCCATCAGAGGA
CGCACTGGACAGTCTCGTAGGGCAAGCACTATTTGGTGATGGAAGCGCTGCGGTC
ATTGTTGGTAGCGACCCAGACATATCAATTGAAAGACCTCTTTTCCAACTTGTCTC
TGCTGCCCAAACTTTTATTCCGAATAGCGCCGGGGCTATCGCGGGTAATCTTAGAG
AAGTGGGACTGACGTTTCATTTATGGCCAAATGTGCCCACACTTATAAGCGAAAAT
GTCGAAAAATGTCTTACGCAGGCATTCGATCCTCTTGGTATATCGGATTGGAACTC
TCTCTTTTGGATCGCCCATCCAGGTGGTCCTGCAATTCTGGATGCTGTAGAAGCAA
AACTAAACCTGGACAAGAAGAAACTGGAAGCTACAAGACATGTCTTGTCGGAATA
CGGGAACATGAGTTCGGCATGTGTACTTTTTATTTTAGATGAGATGCGTAAAAAGT
CTCTGAAAGGTGAGCGTGCAACAACCGGTGAAGGTTTGGACTGGGGTGTCTTGTTC
GGATTCGGTCCCGGCTTAACCATCGAAACTGTAGTTCTACATTCTATTCCAATGGT
TACTAATTAA
Synthetic DNA
Codon optimized Vitis vinifera stilbene synthase opt5
SEQ ID NO: 24
ATGGCTTCAGTCGAGGAGTTTAGAAATGCTCAGAGGGCCAAGGGTCCTGCCACAA
TATTAGCTATAGGTACTGCCACCCCAGATCACTGTGTCTATCAAAGTGACTATGCT
GACTATTATTTTAAAGTCACAAAAAGTGAGCACATGACTGCATTGAAAAAGAAAT
TCAATAGGATATGTGATAAATCAATGATCAAAAAGAGATACATTCATCTAACTGA
GGAAATGTTAGAAGAGCATCCAAATATTGGTGCATATATGGCTCCATCCTTAAATA
TCAGACAGGAAATAATAACCGCTGAGGTGCCTAAACTGGGTAAAGAAGCTGCATT
AAAAGCATTAAAAGAATGGGGTCAGCCTAAATCAAAGATTACGCATCTAGTATTT
TGCACAACGTCTGGTGTCGAAATGCCTGGAGCCGATTACAAACTAGCAAATTTACT
AGGTCTTGAACCTTCTGTCCGTCGAGTAATGTTATACCACCAAGGTTGCTACGCAG
GCGGAACCGTTCTAAGGACTGCCAAGGACTTGGCAGAAAATAACGCTGGTGCAAG
GGTTTTAGTGGTTTGTTCTGAAATCACTGTAGTCACATTTAGGGGTCCCTCTGAAG
ATGCATTAGACTCTTTAGTTGGGCAAGCACTGTTCGGGGATGGGTCTGCGGCCGTT
ATAGTAGGTTCAGATCCTGACATTTCTATCGAAAGGCCTCTGTTTCAACTGGTATC
TGCTGCCCAAACTTTTATTCCTAACAGCGCTGGTGCAATCGCCGGGAACCTCCGAG
AAGTAGGTCTTACATTTCATCTATGGCCTAATGTCCCTACTTTGATTTCCGAGAATG
TAGAGAAATGCCTGACTCAGGCCTTTGATCCTTTGGGCATATCTGATTGGAACTCA
CTATTTTGGATTGCACACCCCGGAGGTCCCGCAATTTTGGATGCCGTGGAGGCTAA
ATTAAATTTAGATAAGAAGAAACTCGAAGCAACTAGACATGTATTATCAGAGTAC
GGCAATATGTCTAGTGCTTGTGTTTTATTTATTTTAGACGAAATGCGTAAAAAGTC
TTTAAAGGGAGAGAGGGCTACTACAGGAGAAGGATTAGATTGGGGTGTTTTGTTT
GGTTTCGGACCCGGTTTAACGATCGAAACAGTTGTTCTGCATAGTATCCCTATGGT
GACCAATTGA
Synthetic DNA
Codon optimized Vitis vinifera stilbene synthase opt6
SEQ ID NO: 25
ATGGCATCGGTAGAAGAGTTCAGAAATGCACAGAGGGCTAAAGGCCCTGCCACAA
TCCTAGCAATTGGTACTGCAACTCCCGATCATTGCGTTTATCAAAGTGATTATGCC
GACTATTATTTTAAAGTTACGAAATCAGAACACATGACTGCTCTTAAAAAGAAATT
CAACAGAATATGTGACAAGAGTATGATTAAAAAGAGATACATTCACTTGACAGAA
GAGATGTTGGAGGAGCATCCTAATATCGGCGCTTACATGGCACCTTCATTGAACAT
TCGTCAAGAAATAATTACTGCCGAGGTTCCTAAACTCGGCAAAGAAGCAGCACTT
AAGGCACTTAAGGAATGGGGTCAGCCAAAGTCAAAGATCACACATTTGGTCTTTT
GTACAACCTCTGGAGTTGAGATGCCAGGCGCTGATTATAAATTGGCTAATCTTTTA
GGATTAGAGCCAAGTGTTAGGCGGGTGATGCTATATCACCAAGGTTGTTATGCAG
GTGGTACTGTTTTGAGGACAGCCAAGGATCTGGCCGAAAATAATGCTGGGGCCAG
AGTCCTGGTTGTTTGCTCCGAGATAACTGTTGTTACATTTCGCGGGCCTTCAGAAG
ATGCACTGGATTCTCTTGTGGGACAGGCGCTGTTTGGTGATGGGTCCGCTGCCGTG
ATCGTAGGCTCTGATCCAGATATATCAATTGAGAGGCCTTTATTTCAGTTGGTGTC
TGCCGCTCAGACATTCATCCCTAATTCCGCGGGAGCGATAGCTGGTAATCTAAGAG
AGGTTGGCTTGACATTTCACTTATGGCCTAATGTGCCAACATTGATCTCTGAGAAC
GTCGAAAAGTGCCTAACCCAAGCATTTGACCCATTAGGAATTAGCGACTGGAATA
GTTTATTTTGGATAGCACACCCTGGAGGTCCGGCTATATTGGATGCTGTGGAAGCA
AAGCTAAATCTGGATAAGAAGAAGCTAGAAGCAACAAGACACGTACTATCTGAAT
ACGGAAATATGAGCAGTGCTTGTGTTCTATTTATTCTTGATGAGATGCGTAAAAAG
AGTTTAAAtGGAGAAAGAGCCACCACAGGTGAAGGGCTAGACTGGGGCGTTTTATT
TGGCTTCGGTCCAGGTCTGACAATCGAAACGGTCGTCTTACACTCAATTCCAATGG
TTACAAATTGA
Amino acids
PDC5
SEQ ID NO: 26
MSEITLGKYLFERLSQVNCNTVFGLPGDFNLSLLDKLYEVKGMRWAGNANELNAAY
AADGYARIKGMSCIITTFGVGELSALNGIAGSYAEHVGVLHVVGVPSISSQAKQLLLH
HTLGNGDFTVFHRMSANISETTAMITDIANAPAEIDRCIRTTYTTQRPVYLGLPANLVD
LNVPAKLLETPIDLSLKPNDAEAEAEVVRTVVELIKDAKNPVILADACASRHDVKAET
KKLMDLTQFPVYVTPMGKGAIDEQHPRYGGVYVGTLSRPEVKKAVESADLILSIGALL
SDFNTGSFSYSYKTKNIVEFHSDHIKIRNATFPGVQMKFALQKLLDAIPEVVKDYKPVA
VPARVPITKSTPANTPMKQEWMWNHLGNFLREGDIVIAETGTSAFGINQTTFPTDVYA
IVQVLWGSIGFTVGALLGATMAAEELDPKKRVILFIGDGSLQLTVQEISTMIRWGLKPY
IFVLNNNGYTIEKLIHGPHAEYNEIQGWDHLALLPTFGARNYETHRVATTGEWEKLTQ
DKDFQDNSKIRMIEVMLPVFDAPQNLVKQAQLTAATNAKQ
DNA
PDC5
SEQ ID NO: 27
ATGTCTGAAATAACCTTAGGTAAATATTTATTTGAAAGATTGAGCCAAGTCAACTG
TAACACCGTCTTCGGTTTGCCAGGTGACTTTAACTTGTCTCTTTTGGATAAGCTTTA
TGAAGTCAAAGGTATGAGATGGGCTGGTAACGCTAACGAATTGAACGCTGCCTAT
GCTGCTGATGGTTACGCTCGTATCAAGGGTATGTCCTGTATTATTACCACCTTCGGT
GTTGGTGAATTGTCTGCTTTGAATGGTATTGCCGGTTCTTACGCTGAACATGTCGGT
GTTTTGCACGTTGTTGGTGTTCCATCCATCTCTTCTCAAGCTAAGCAATTGTTGTTG
CATCATACCTTGGGTAACGGTGACTTCACTGTTTTCCACAGAATGTCTGCCAACAT
TTCTGAAACCACTGCCATGATCACTGATATTGCTAACGCTCCAGCTGAAATTGACA
GATGTATCAGAACCACCTACACTACCCAAAGACCAGTCTACTTGGGTTTGCCAGCT
AACTTGGTTGACTTGAACGTCCCAGCCAAGTTATTGGAAACTCCAATTGACTTGTC
TTTGAAGCCAAACGACGCTGAAGCTGAAGCTGAAGTTGTTAGAACTGTTGTTGAAT
TGATCAAGGATGCTAAGAACCCAGTTATCTTGGCTGATGCTTGTGCTTCTAGACAT
GATGTCAAGGCTGAAACTAAGAAGTTGATGGACTTGACTCAATTCCCAGTTTACGT
CACCCCAATGGGTAAGGGTGCTATTGACGAACAACACCCAAGATACGGTGGTGTT
TACGTTGGTACCTTGTCTAGACCAGAAGTTAAGAAGGCTGTAGAATCTGCTGATTT
GATATTGTCTATCGGTGCTTTGTTGTCTGATTTCAATACCGGTTCTTTCTCTTACTCC
TACAAGACCAAAAATATCGTTGAATTCCACTCTGACCACATCAAGATCAGAAACG
CCACCTTCCCAGGTGTTCAAATGAAATTTGCCTTGCAAAAATTGTTGGATGCTATT
CCAGAAGTCGTCAAGGACTACAAACCTGTTGCTGTCCCAGCTAGAGTTCCAATTAC
CAAGTCTACTCCAGCTAACACTCCAATGAAGCAAGAATGGATGTGGAACCATTTG
GGTAACTTCTTGAGAGAAGGTGATATTGTTATTGCTGAAACCGGTACTTCCGCCTT
CGGTATTAACCAAACTACTTTCCCAACAGATGTATACGCTATCGTCCAAGTCTTGT
GGGGTTCCATTGGTTTCACAGTCGGCGCTCTATTGGGTGCTACTATGGCCGCTGAA
GAACTTGATCCAAAGAAGAGAGTTATTTTATTCATTGGTGACGGTTCTCTACAATT
GACTGTTCAAGAAATCTCTACCATGATTAGATGGGGTTTGAAGCCATACATTTTTG
TCTTGAATAACAACGGTTACACCATTGAAAAATTGATTCACGGTCCTCATGCCGAA
TATAATGAAATTCAAGGTTGGGACCACTTGGCCTTATTGCCAACTTTTGGTGCTAG
AAACTACGAAACCCACAGAGTTGCTACCACTGGTGAATGGGAAAAGTTGACTCAA
GACAAGGACTTCCAAGACAACTCTAAGATTAGAATGATTGAAGTTATGTTGCCAG
TCTTTGATGCTCCACAAAACTTGGTTAAACAAGCTCAATTGACTGCCGCTACTAAC
GCTAAACAATAA
DNA
TDH3 promoter
SEQ ID NO: 28
TCATTATCAATACTGCCATTTCAAAGAATACGTAAATAATTAATAGTAGTGATTTT
CCTAACTTTATTTAGTCAAAAAATTAGCCTTTTAATTCTGCTGTAACCCGTACATGC
CCAAAATAGGGGGCGGGTTACACAGAATATATAACATCGTAGGTGTCTGGGTGAA
CAGTTTATTCCTGGCATCCACTAAATATAATGGAGCCCGCTTTTTAAGCTGGCATC
CAGAAAAAAAAAGAATCCCAGCACCAAAATATTGTTTTCTTCACCAACCATCAGT
TCATAGGTCCATTCTCTTAGCGCAACTACAGAGAACAGGGGCACAAACAGGCAAA
AAACGGGCACAACCTCAATGGAGTGATGCAACCTGCCTGGAGTAAATGATGACAC
AAGGCAATTGACCCACGCATGTATCTATCTCATTTTCTTACACCTTCTATTACCTTC
TGCTCTCTCTGATTTGGAAAAAGCTGAAAAAAAAGGTTGAAACCAGTTCCCTGAA
ATTATTCCCCTACTTGACTAATAAGTATATAAAGACGGTAGGTATTGATTGTAATT
CTGTAAATCTATTTCTTAAACTTCTTAAATTCTACTTTTATAGTTAGTCTTTTTTTTA
GTTTTAAAACACCAAGAACTTAGTTTCGAATAAACACACATAAACAAACAAA
Amino acid
Modified Saccharomyces cerevisiae acetyl CoA carboxylase 1
SEQ ID NO: 29
MSEESLFESSPQKMEYEITNYSERHTELPGHFIGLNTVDKLEESPLRDFVKSHGGHTVIS
KILIANNGIAAVKEIRSVRKWAYETFGDDRTVQFVAMATPEDLEANAEYIRMADQYIE
VPGGTNNNNYANVDLIVDIAERADVDAVWAGWGHASENPLLPEKLSQSKRKVIFIGP
PGNAMRSLGDKISSTIVAQSAKVPCIPWSGTGVDTVHVDEKTGLVSVDDDIYQKGCCT
SPEDGLQKAKRIGFPVMIKASEGGGGKGIRQVEREEDFIALYHQAANEIPGSPIFIMKLA
GRARHLEVQLLADQYGTNISLFGRDCSVQRRHQKIIEEAPVTIAKAETFHEMEKAAVR
LGKLVGYVSAGTVEYLYSHDDGKFYFLELNPRLQVEHPTTEMVSGVNLPAAQLQIAM
GIPMHRISDIRTLYGMNPHSASEIDFEFKTQDATKKQRRPIPKGHCTACRITSEDPNDGF
KPSGGTLHELNFRSSSNVWGYFSVGNNGNIHSFSDSQFGHIFAFGENRQASRKHMVVA
LKELSIRGDFRTTVEYLIKLLETEDFEDNTITTGWLDDLITHKMTAEKPDPTLAVICGAA
TKAFLASEEARHKYIESLQKGQVLSKDLLQTMFPVDFIHEGKRYKFTVAKSGNDRYTL
FINGSKCDIILRQLADGGLLIAIGGKSHTIYWKEEVAATRLSVDSMTTLLEVENDPTQL
RTPSPGKLVKFLVENGEHIIKGQPYAEIEVMKMQMPLVSQENGIVQLLKQPGSTIVAG
DIMAIMTLDDPSKVKHALPFEGMLPDFGSPVIEGTKPAYKFKSLVSTLENILKGYDNQ
VIMNASLQQLIEVLRNPKLPYSEWKLHISALHSRLPAKLDEQMEELVARSLERGAVFP
ARQLSKLIDMAVKNPEYNPDKLLGAVVEPLADIAHKYSNGLEAHEHSIFVHFLEEYYE
VEKLFNGPNVREENIILKLRDENPKDLDKVALTVLSHSKVSAKNNLILAILKHYQPLCK
LSSKVSAIFSTPLQHIVELESKATAKVALQAREILIQGALPSVKERTEQIEHILKSSVVKV
AYGSSNPKRSEPDLNILKDLIDSNYVVFDVLLQFLTHQDPVVTAAAAQVYIRRAYRAY
TIGDIRVHEGVTVPIVEWKFQLPSAAFSTFPTVKSKMGMNRAVAVSDLSYVANSQSSP
LREGILMAVDHLDDVDEILSQSLEVIPRHQSSSNGPAPDRSGSSASLSNVANVCVASTE
GFESEEEILVRLREILDLNKQELINASIRRITFMFGFKDGSYPKYYTFNGPNYNENETIRH
IEPALAFQLELGRLSNFNIKPIFTDNRNIHVYEAVSKTSPLDKRFFTRGIIRTGHIRDDISI
QEYLTSEANRLMSDILDNLEVTDTSNSDLNHIFINFIAVFDISPEDVEAAFGGFLERFGK
RLLRLRVSSAEIRIIIKDPQTGAPVPLRALINNVSGYVIKTEMYTEVKNAKGEWVFKSL
GKPGSMHLRPIATPYPVKEWLQPKRYKAHLMGTTYVYDFPELFRQASSSQWKNFSAD
VKLTDDFFISNELIEDENGELTEVEREPGANAIGMVAFKITVKTPEYPRGRQFVVVAND
ITFKIGSFGPQEDEFFNKVTEYARKRGIPRIYLAANSGARIGMAEEIVPLFQVAWNDAA
NPDKGFQYLYLTSEGMETLKKFDKENSVLTERTVINGEERFVIKTIIGSEDGLGVECLR
GSGLIAGATSRAYHDIFTITLVTCRSVGIGAYLVRLGQRAIQVEGQPIILTGAPAINKML
GREVYTSNLQLGGTQIMYNNGVSHLTAVDDLAGVEKIVEWMSYVPAKRNMPVPILET
KDTWDRPVDFTPTNDETYDVRWMIEGRETESGFEYGLFDKGSFFETLSGWAKGVVV
GRARLGGIPLGVIGVETRTVENLIPADPANPNSAETLIQEPGQVWHPNSAFKTAQAIND
FNNGEQLPMMILANWRGFSGGQRDMFNEVLKYGSFIVDALVDYKQPIIIYIPPTGELRG
GSWVVVDPTINADQMEMYADVNARAGVLEPQGMVGIKFRREKLLDTMNRLDDKYR
ELRSQLSNKSLAPEVHQQISKQLADRERELLPIYGQISLQFADLHDRSSRMVAKGVISK
ELEWTEARRFFFWRLRRRLNEEYLIKRLSHQVGEASRLEKIARIRSWYPASVDHEDDR
QVATWIEENYKTLDDKLKGLKLESFAQDLAKKIRSDHDNAIDGLSEVIKMLSTDDKEK
LLKTLK
DNA
Modified Saccharomyces cerevisiae acetyl CoA carboxylase 1
SEQ ID NO: 30
ATGAGCGAAGAAAGCTTATTCGAGTCTTCTCCACAGAAGATGGAGTACGAAATTA
CAAACTACTCAGAAAGACATACAGAACTTCCAGGTCATTTCATTGGCCTCAATACA
GTAGATAAACTAGAGGAGTCCCCGTTAAGGGACTTTGTTAAGAGTCACGGTGGTC
ACACGGTCATATCCAAGATCCTGATAGCAAATAATGGTATTGCCGCCGTGAAAGA
AATTAGATCCGTCAGAAAATGGGCATACGAGACGTTCGGCGATGACAGAACCGTC
CAATTCGTCGCCATGGCCACCCCAGAAGATCTGGAGGCCAACGCAGAATATATCC
GTATGGCCGATCAATACATTGAAGTGCCAGGTGGTACTAATAATAACAACTACGC
TAACGTAGACTTGATCGTAGACATCGCCGAAAGAGCAGACGTAGACGCCGTATGG
GCTGGCTGGGGTCACGCCTCCGAGAATCCACTATTGCCTGAAAAATTGTCCCAGTC
TAAGAGGAAAGTCATCTTTATTGGGCCTCCAGGTAACGCCATGAGGTCTTTAGGTG
ATAAAATCTCCTCTACCATTGTCGCTCAAAGTGCTAAAGTCCCATGTATTCCATGG
TCTGGTACCGGTGTTGACACCGTTCACGTGGACGAGAAAACCGGTCTGGTCTCTGT
CGACGATGACATCTATCAAAAGGGTTGTTGTACCTCTCCTGAAGATGGTTTACAAA
AGGCCAAGCGTATTGGTTTTCCTGTCATGATTAAGGCATCCGAAGGTGGTGGTGGT
AAAGGTATCAGACAAGTTGAACGTGAAGAAGATTTCATCGCTTTATACCACCAGG
CAGCCAACGAAATTCCAGGCTCCCCCATTTTCATCATGAAGTTGGCCGGTAGAGCG
CGTCACTTGGAAGTTCAACTGCTAGCAGATCAGTACGGTACAAATATTTCCTTGTT
CGGTAGAGACTGTTCCGTTCAGAGACGTCATCAAAAAATTATCGAAGAAGCACCA
GTTACAATTGCCAAGGCTGAAACATTTCACGAGATGGAAAAGGCTGCCGTCAGAC
TGGGGAAACTAGTCGGTTATGTCTCTGCCGGTACCGTGGAGTATCTATATTCTCAT
GATGATGGAAAATTCTACTTTTTAGAATTGAACCCAAGATTACAAGTCGAGCATCC
AACAACGGAAATGGTCTCCGGTGTTAACTTACCTGCAGCTCAATTACAAATCGCTA
TGGGTATCCCTATGCATAGAATAAGTGACATTAGAACTTTATATGGTATGAATCCT
CATTCTGCCTCAGAAATCGATTTCGAATTCAAAACTCAAGATGCCACCAAGAAAC
AAAGAAGACCTATTCCAAAGGGTCATTGTACCGCTTGTCGTATCACATCAGAAGAT
CCAAACGATGGATTCAAGCCATCGGGTGGTACTTTGCATGAACTAAACTTCCGTTC
TTCCTCTAATGTTTGGGGTTACTTCTCCGTGGGTAACAATGGTAATATTCACTCCTT
TTCGGACTCTCAGTTCGGCCATATTTTTGCTTTTGGTGAAAATAGACAAGCTTCCA
GGAAACACATGGTTGTTGCCCTGAAGGAATTGTCCATTAGGGGTGATTTCAGAACT
ACTGTGGAATACTTGATCAAACTTTTGGAAACTGAAGATTTCGAGGATAACACTAT
TACCACCGGTTGGTTGGACGATTTGATTACTCATAAAATGACCGCTGAAAAGCCTG
ATCCAACTCTTGCCGTCATTTGCGGTGCCGCTACAAAGGCTTTCTTAGCATCTGAA
GAAGCCCGCCACAAGTATATCGAATCCTTACAAAAGGGACAAGTTCTATCTAAAG
ACCTACTGCAAACTATGTTCCCTGTAGATTTTATCCATGAGGGTAAAAGATACAAG
TTCACCGTAGCTAAATCCGGTAATGACCGTTACACATTATTTATCAATGGTTCTAA
ATGTGATATCATACTGCGTCAACTAGCTGATGGTGGTCTTTTGATTGCCATAGGCG
GTAAATCGCATACCATCTATTGGAAAGAAGAAGTTGCTGCTACAAGATTATCCGTT
GACTCTATGACTACTTTGTTGGAAGTTGAAAACGATCCAACCCAGTTGCGTACTCC
ATCCCCTGGTAAATTGGTTAAATTCTTGGTGGAAAATGGTGAACACATTATCAAGG
GCCAACCATATGCAGAAATTGAAGTTATGAAAATGCAAATGCCTTTGGTTTCTCAA
GAAAATGGTATCGTCCAGTTATTAAAGCAACCTGGTTCTACCATTGTTGCAGGTGA
TATCATGGCTATTATGACTCTTGACGATCCATCCAAGGTCAAGCACGCTCTACCAT
TTGAAGGTATGCTGCCAGATTTTGGTTCTCCAGTTATCGAAGGAACCAAACCTGCC
TATAAATTCAAGTCATTAGTGTCTACTTTGGAAAACATTTTGAAGGGTTATGACAA
CCAAGTTATTATGAACGCTTCCTTGCAACAATTGATAGAGGTTTTGAGAAATCCAA
AACTGCCTTACTCAGAATGGAAACTACACATCTCTGCTTTACATTCAAGATTGCCT
GCTAAGCTAGATGAACAAATGGAAGAGTTAGTTGCACGTTCTTTGAGACGTGGTG
CTGTTTTCCCAGCTAGACAATTAAGTAAATTGATTGATATGGCCGTGAAGAATCCT
GAATACAACCCCGACAAATTGCTGGGCGCCGTCGTGGAACCATTGGCGGATATTG
CTCATAAGTACTCTAACGGGTTAGAAGCCCATGAACATTCTATATTTGTCCATTTCT
TGGAAGAATATTACGAAGTTGAAAAGTTATTCAATGGTCCAAATGTTCGTGAGGA
AAATATCATTCTGAAATTGCGTGATGAAAACCCTAAAGATCTAGATAAAGTTGCG
CTAACTGTTTTGTCTCATTCGAAAGTTTCAGCGAAGAATAACCTGATCCTAGCTAT
CTTGAAACATTATCAACCATTGTGCAAGTTATCTTCTAAAGTTTCTGCCATTTTCTC
TACTCCTCTACAACATATTGTTGAACTAGAATCTAAGGCTACCGCTAAGGTCGCTC
TACAAGCAAGAGAAATTTTGATTCAAGGCGCTTTACCTTCGGTCAAGGAAAGAAC
TGAACAAATTGAACATATCTTAAAATCCTCTGTTGTGAAGGTTGCCTATGGCTCAT
CCAATCCAAAGCGCTCTGAACCAGATTTGAATATCTTGAAGGACTTGATCGATTCT
AATTACGTTGTGTTCGATGTTTTACTTCAATTCCTAACCCATCAAGACCCAGTTGTG
ACTGCTGCAGCTGCTCAAGTCTATATTCGTCGTGCTTATCGTGCTTACACCATAGG
AGATATTAGAGTTCACGAAGGTGTCACAGTTCCAATTGTTGAATGGAAATTCCAAC
TACCTTCAGCTGCGTTCTCCACCTTTCCAACTGTTAAATCTAAAATGGGTATGAAC
AGGGCTGTTGCTGTTTCAGATTTGTCATATGTTGCAAACAGTCAGTCATCTCCGTTA
AGAGAAGGTATTTTGATGGCTGTGGATCATTTAGATGATGTTGATGAAATTTTGTC
ACAAAGTTTGGAAGTTATTCCTCGTCACCAATCTTCTTCTAACGGACCTGCTCCTG
ATCGTTCTGGTAGCTCCGCATCGTTGAGTAATGTTGCTAATGTTTGTGTTGCTTCTA
CAGAAGGTTTCGAATCTGAAGAGGAAATTTTGGTAAGGTTGAGAGAAATTTTGGA
TTTGAATAAGCAGGAATTAATCAATGCTTCTATCCGTCGTATCACATTTATGTTCG
GTTTTAAAGATGGGTCTTATCCAAAGTATTATACTTTTAACGGTCCAAATTATAAC
GAAAATGAAACAATTCGTCACATTGAGCCGGCTTTGGCCTTCCAACTGGAATTAGG
AAGATTGTCCAACTTCAACATTAAACCAATTTTCACTGATAATAGAAACATCCATG
TCTACGAAGCTGTTAGTAAGACTTCTCCATTGGATAAGAGATTCTTTACAAGAGGT
ATTATTAGAACGGGTCATATCCGTGATGACATTTCTATTCAAGAATATCTGACTTC
TGAAGCTAACAGATTGATGAGTGATATATTGGATAATTTAGAAGTCACCGACACTT
CAAATTCTGATTTGAATCATATCTTCATCAACTTCATTGCGGTGTTTGATATCTCTC
CAGAAGATGTCGAAGCCGCCTTCGGTGGTTTCTTAGAAAGATTTGGTAAGAGATTG
TTGAGATTGCGTGTTTCTTCTGCCGAAATTAGAATCATCATCAAAGATCCTCAAAC
AGGTGCCCCAGTACCATTGCGTGCCTTGATCAATAACGTTTCTGGTTATGTTATCA
AAACAGAAATGTACACCGAAGTCAAGAACGCAAAAGGTGAATGGGTATTTAAGTC
TTTGGGTAAACCTGGATCCATGCATTTAAGACCTATTGCTACTCCTTACCCTGTTAA
GGAATGGTTGCAACCAAAACGTTATAAGGCACACTTGATGGGTACCACATATGTC
TATGACTTCCCAGAATTATTCCGCCAAGCATCGTCATCCCAATGGAAAAATTTCTC
TGCAGATGTTAAGTTAACAGATGATTTCTTTATTTCCAACGAGTTGATTGAAGATG
AAAACGGCGAATTAACTGAGGTGGAAAGAGAACCTGGTGCCAACGCTATTGGTAT
GGTTGCCTTTAAGATTACTGTAAAGACTCCTGAATATCCAAGAGGCCGTCAATTTG
TTGTTGTTGCTAACGATATCACATTCAAGATCGGTTCCTTTGGTCCACAAGAAGAC
GAATTCTTCAATAAGGTTACTGAATATGCTAGAAAGCGTGGTATCCCAAGAATTTA
CTTGGCTGCAAACTCAGGTGCCAGAATTGGTATGGCTGAAGAGATTGTTCCACTAT
TTCAAGTTGCATGGAATGATGCTGCCAATCCGGACAAGGGCTTCCAATACTTATAC
TTAACAAGTGAAGGTATGGAAACTTTAAAGAAATTTGACAAAGAAAATTCTGTTC
TCACTGAACGTACTGTTATAAACGGTGAAGAAAGATTTGTCATCAAGACAATTATT
GGTTCTGAAGATGGGTTAGGTGTCGAATGTCTACGTGGATCTGGTTTAATTGCTGG
TGCAACGTCAAGGGCTTACCACGATATCTTCACTATCACCTTAGTCACTTGTAGAT
CCGTCGGTATCGGTGCTTATTTGGTTCGTTTGGGTCAAAGAGCTATTCAGGTCGAA
GGCCAGCCAATTATTTTAACTGGTGCTCCTGCAATCAACAAAATGCTGGGTAGAGA
AGTTTATACTTCTAACTTACAATTGGGTGGTACTCAAATCATGTATAACAACGGTG
TTTCACATTTGACTGCTGTTGACGATTTAGCTGGTGTAGAGAAGATTGTTGAATGG
ATGTCTTATGTTCCAGCCAAGCGTAATATGCCAGTTCCTATCTTGGAAACTAAAGA
CACATGGGATAGACCAGTTGATTTCACTCCAACTAATGATGAAACTTACGATGTAA
GATGGATGATTGAAGGTCGTGAGACTGAAAGTGGATTTGAATATGGTTTGTTTGAT
AAAGGGTCTTTCTTTGAAACTTTGTCAGGATGGGCCAAAGGTGTTGTCGTTGGTAG
AGCCCGTCTTGGTGGTATTCCACTGGGTGTTATTGGTGTTGAAACAAGAACTGTCG
AGAACTTGATTCCTGCTGATCCAGCTAATCCAAATAGTGCTGAAACATTAATTCAA
GAACCTGGTCAAGTTTGGCATCCAAACTCCGCCTTCAAGACTGCTCAAGCTATCAA
TGACTTTAACAACGGTGAACAATTGCCAATGATGATTTTGGCCAACTGGAGAGGTT
TCTCTGGTGGTCAACGTGATATGTTCAACGAAGTCTTGAAGTATGGTTCGTTTATT
GTTGACGCATTGGTGGATTACAAACAACCAATTATTATCTATATCCCACCTACCGG
TGAACTAAGAGGTGGTTCATGGGTTGTTGTCGATCCAACTATCAACGCTGACCAAA
TGGAAATGTATGCCGACGTCAACGCTAGAGCTGGTGTTTTGGAACCACAAGGTAT
GGTTGGTATCAAGTTCCGTAGAGAAAAATTGCTGGACACCATGAACAGATTGGAT
GACAAGTACAGAGAATTGAGATCTCAATTATCCAACAAGAGTTTGGCTCCAGAAG
TACATCAGCAAATATCCAAGCAATTAGCTGATCGTGAGAGAGAACTATTGCCAAT
TTACGGACAAATCAGTCTTCAATTTGCTGATTTGCACGATAGGTCTTCACGTATGG
TGGCCAAGGGTGTTATTTCTAAGGAACTGGAATGGACCGAGGCACGTCGTTTCTTC
TTCTGGAGATTGAGAAGAAGATTGAACGAAGAATATTTGATTAAAAGGTTGAGCC
ATCAGGTAGGCGAAGCATCAAGATTAGAAAAGATCGCAAGAATTAGATCGTGGTA
CCCTGCTTCAGTGGACCATGAAGATGATAGGCAAGTCGCAACATGGATTGAAGAA
AACTACAAAACTTTGGACGATAAACTAAAGGGTTTGAAATTAGAGTCATTCGCTC
AAGACTTAGCTAAAAAGATCAGAAGCGACCATGACAATGCTATTGATGGATTATC
TGAAGTTATCAAGATGTTATCTACCGATGATAAAGAAAAATTGTTGAAGACTTTGA
AATAA

What is claimed is:

1. A method of increasing production of a dihydrodiphenylpropanoid derivative compound in a recombinant host cell, the method comprising:
reducing or eliminating expression of a gene encoding a double-bond reductase polypeptide or activity of the double-bond reductase polypeptide encoded thereby; and
expressing a recombinant gene encoding a double-bond reductase selected from the group consisting of a first polypeptide, second polypeptide, third polypeptide, and combinations thereof,
wherein the first polypeptide comprises an amino acid sequence having at least 90% identity to the amino acid sequence as set forth in SEQ ID NO: 3, the second polypeptide comprises an amino acid sequence having at least 90% identity to the amino acid sequence as set forth in SEQ ID NO: 5, and the third polypeptide comprises an amino acid sequence having at least 90% identity to the amino acid sequence as set forth in SEQ ID NO: 7,
wherein the product dihydrophenylpropanoid derivative compound is a dihydrostilbenoid or a dihydrochalcone compound.

2. The method according to claim 1, wherein the first polypeptide comprises an amino acid sequence having at least 95%, at least 99%, or 100% identity to the amino acid sequence as set forth in SEQ ID NO: 3.

3. The method according to claim 1, wherein the first polypeptide comprises an amino acid sequence having at least 95%, at least 99%, or 100% identity to the amino acid sequence as set forth in SEQ ID NO: 5.

4. The method according to claim 1, wherein the first polypeptide comprises an amino acid sequence having at least 95%, at least 99%, or 100% identity to the amino acid sequence as set forth in SEQ ID NO: 7.

5. The method according to any of claims 1 to 3, wherein the gene whose expression is reduced or eliminated encodes an enoyl reductase.

6. The method according to claim 5, wherein the enoyl reductase is a S. cerevisiae trans-2-enoyl-CoA reductase TSC13.

7. The method according to claim 5 or 6, wherein expression of the enoyl reductase gene is eliminated by deletion of the gene.

8. The method according to any of claims 1 to 7, wherein the dihydrostilbenoid is dihydroresveratrol, and the dihydrochalcone compound is phloretin.

9. The method according to any of claim 1 to 8, wherein the recombinant host cell is a yeast cell.

10. The method according to claim 9, wherein the yeast is a cell from the Saccharomyces cerevisiae species.

11. A recombinant host cell capable of producing a dihydrodiphenylpropanoid compound, comprising:
a gene encoding a double-bond reductase polypeptide, wherein expression of the gene or activity of the double-bond reductase polypeptide encoded thereby is reduced or eliminated; and
a recombinant gene encoding a double-bond reductase selected from the group consisting of a first polypeptide, second polypeptide, third polypeptide, and combinations thereof,
wherein the first polypeptide comprises an amino acid sequence having at least 90% identity to the amino acid sequence as set forth in SEQ ID NO: 3, the second polypeptide comprises an amino acid sequence having at least 90% identity to the amino acid sequence as set forth in SEQ ID NO: 5, and the third polypeptide comprises an amino acid sequence having at least 90% identity to the amino acid sequence as set forth in SEQ ID NO: 7.

12. The recombinant host cell according to claim 11, wherein the first polypeptide comprises an amino acid sequence having at least 95%, at least 99%, or 100% identity to the amino acid sequence as set forth in SEQ ID NO: 3.

13. The recombinant host cell according to claim 11, wherein the first polypeptide comprises an amino acid sequence having at least 95%, at least 99%, or 100% identity to the amino acid sequence as set forth in SEQ ID NO: 5.

14. The recombinant host cell according to claim 11, wherein the first polypeptide comprises an amino acid sequence having at least 95%, at least 99%, or 100% identity to the amino acid sequence as set forth in SEQ ID NO: 7.

15. The recombinant host cell according to any of claims 11 to 14, wherein the gene whose expression is reduced or eliminated encodes an enoyl reductase.

16. The recombinant host cell according to claim 11, wherein the enoyl reductase is a S. cerevisiae trans-2-enoyl-CoA reductase TSC13.

17. The recombinant host cell according to claim 15 or 16, wherein expression of the enoyl reductase gene is eliminated by deletion of the gene.

18. The recombinant host cell according to any of claims 11 to 17, wherein the dihydrostilbenoid is dihydroresveratrol, and the dihydrochalcone compound is phloretin.

19. The recombinant host cell according to any of claims 11 to 18, wherein the recombinant host cell is a yeast cell.

20. The recombinant host cell according to claim 19, wherein the yeast cell is a Saccharomycete cell.

21. The recombinant host cell according to claim 20, wherein the yeast cell is a cell from the Saccharomyces cerevisiae species.

22. A method of producing a dihydrochalcone compound or a dihydrostilbenoid compound, the method comprising cultivating a recombinant host cell according to any of claims 11 to 21 in a culture medium under conditions in which the recombinant genes are expressed, and wherein the product compound is synthesized by the recombinant host cell.

23. The method according to claim 22, wherein the product compound is a dihydrochalcone compound.

24. The method according to claim 23, wherein the dihydrochalcone compound is phloretin.

25. The method according to claim 23, wherein the product compound is a dihydrostilbenoid compound.

26. The method according to claim 25, wherein the dihydrostilbenoid compound is dihydrostilbene.

27. A method of producing a compound of formula (V): 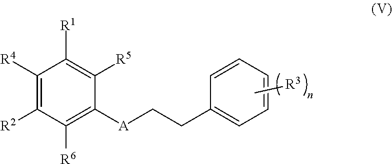
or a pharmaceutically acceptable salt thereof, wherein
A is a bond or C═O;
n is an integer 0, 1, 2, 3, or 4;
R¹ is hydrogen or —OR¹¹;
wherein each R¹¹ is independently hydrogen, C₁-C₆ alkyl, or glycosyl;
R² is hydrogen, —OR¹¹, C₁-C₁₂ alkyl, or C₂-C₁₂ alkenyl, wherein alkyl and alkenyl are optionally substituted with one or more R⁷;
or R² and R⁶ together with the atoms to which they are attached form a 5- to 7-member heterocyclyl optionally substituted with one or more R⁷ groups;
or R² and R⁴ together with the atoms to which they are attached form a 5- to 7-member heterocyclyl optionally substituted with one or more R⁷ groups;
R³ is independently selected from nitro, C₁-C₆ alkyl, C₂-C₆ alkenyl, C₁-C₆ haloalkyl, C₁-C₆ hydroxyalkyl, —OR¹², —N(R¹²)2, —C(O)R¹², —C(O)OR¹², —C(O)N(R¹²)₂, and —S(O)₂R¹², wherein each R¹² is independently hydrogen or C₁-C₆ alkyl;
R⁴ is hydrogen, —OR¹¹, C₁-C₁₂ alkyl, or C₂-C₁₂ alkenyl, wherein alkyl and alkenyl are optionally substituted with one or more R⁷;
or R⁴ and R² together with the atoms to which they are attached form a 5- to 7-member heterocyclyl optionally substituted with one or more R⁷ groups;
R⁵ is hydrogen or —OR¹¹; and
R⁶ is hydrogen, C₁-C₆ alkyl, C₂-C₆ alkenyl, —OR¹¹, or —N(R¹⁰)₂, wherein each R¹⁰ is independently hydrogen or C₁-C₆ alkyl, and wherein alkyl and alkenyl are optionally substituted with one or more R⁸; or R⁶ and R² together with the atoms to which they are attached form a 5- to 7-member heterocyclyl optionally substituted with one or more R⁸ groups;
each R⁷ and R⁸ is independently halogen, cyano, nitro, C₁-C₆ alkyl, C₂-C₆ alkenyl, C₁-C₆ haloalkyl, C₁-C₆ hydroxyalkyl, —OR¹³, —SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl,
the method comprising growing a recombinant host cell of any of claims 11 to 21 in a culture medium under conditions in which the recombinant genes are expressed, and wherein the compound of formula (V) is synthesized by the recombinant host cell.

28. The method according to claim 27, wherein the compound of formula (V) is a dihydrostilbenoid of formula (V-A): 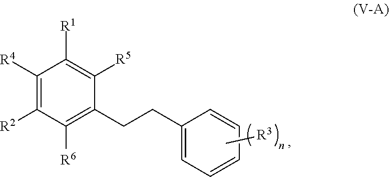
or a pharmaceutically acceptable salt thereof, wherein
n is an integer 0, 1, 2, 3, or 4;
R¹ is hydrogen or —OR¹¹;
wherein each R¹¹ is independently hydrogen, C₁-C₆ alkyl, or glycosyl;
R² is hydrogen, —OR¹¹, C₁-C₁₂ alkyl, or C₂-C₁₂ alkenyl, wherein alkyl and alkenyl are optionally substituted with one or more R⁷;
or R² and R⁶ together with the atoms to which they are attached form a 5- to 7-member heterocyclyl optionally substituted with one or more R⁷ groups;
or R² and R⁴ together with the atoms to which they are attached form a 5- to 7-member heterocyclyl optionally substituted with one or more R⁷ groups;
R³ is independently selected from nitro, C₁-C₆ alkyl, C₂-C₆ alkenyl, C₁-C₆ haloalkyl, C₁-C₆ hydroxyalkyl, —OR¹², —N(R¹²)2, —C(O)R¹², —C(O)OR¹², —C(O)N(R¹²)2, and —S(O)₂R¹², wherein each R¹² is independently hydrogen or C₁-C₆ alkyl;
R⁴ is hydrogen, —OR¹¹, C₁-C₁₂ alkyl, or C₂-C₁₂ alkenyl, wherein alkyl and alkenyl are optionally substituted with one or more R⁷;
or R⁴ and R² together with the atoms to which they are attached form a 5- to 7-member heterocyclyl optionally substituted with one or more R⁷ groups;
R⁵ is hydrogen or —OR¹¹; and
R⁶ is hydrogen, C₁-C₆ alkyl, C₂-C₆ alkenyl, —OR¹¹, or —N(R¹⁰)₂, wherein each R¹⁰ is independently hydrogen or C₁-C₆ alkyl, and wherein alkyl and alkenyl are optionally substituted with one or more R⁸; or R⁶ and R² together with the atoms to which they are attached form a 5- to 7-member heterocyclyl optionally substituted with one or more R⁸ groups;
each R⁷ and R⁸ is independently halogen, cyano, nitro, C₁-C₆ alkyl, C₂-C₆ alkenyl, C₁-C₆haloalkyl, C₁-C₆ hydroxyalkyl, —OR¹³, —SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl.

29. The method according to claim 28, wherein the product dihydrostilbenoid is dihydrostilbene.

30. The method according to claim 27, wherein the compound of formula (V) is a dihydrochalcone compound of formula (V-B): 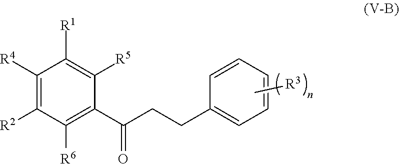
or a pharmaceutically acceptable salt thereof, wherein
n is an integer 0, 1, 2, 3, or 4;
R¹ is hydrogen or —OR¹¹;
wherein each R¹¹ is independently hydrogen, C₁-C₆ alkyl, or glycosyl;
R² is hydrogen, —OR¹¹, C₁-C₁₂ alkyl, or C₂-C₁₂ alkenyl, wherein alkyl and alkenyl are optionally substituted with one or more R⁷;
or R² and R⁶ together with the atoms to which they are attached form a 5- to 7-member heterocyclyl optionally substituted with one or more R⁷ groups;
or R² and R⁴ together with the atoms to which they are attached form a 5- to 7-member heterocyclyl optionally substituted with one or more R⁷ groups;
R³ is independently selected from nitro, C₁-C₆ alkyl, C₂-C₆ alkenyl, C₁-C₆ haloalkyl, C₁-C₆ hydroxyalkyl, —OR¹², —N(R¹²)2, —C(O)R¹², —C(O)OR¹², —C(O)N(R¹²)2, and —S(O)₂R¹², wherein each R¹² is independently hydrogen or C₁-C₆ alkyl;
R⁴ is hydrogen, —OR¹¹, C₁-C₁₂ alkyl, or C₂-C₁₂ alkenyl, wherein alkyl and alkenyl are optionally substituted with one or more R⁷;
or R⁴ and R² together with the atoms to which they are attached form a 5- to 7-member heterocyclyl optionally substituted with one or more R⁷ groups;
R⁵ is hydrogen or —OR¹¹; and
R⁶ is hydrogen, C₁-C₆ alkyl, C₂-C₆ alkenyl, —OR¹¹, or —N(R¹⁰)₂, wherein each R¹⁰ is independently hydrogen or C₁-C₆ alkyl, and wherein alkyl and alkenyl are optionally substituted with one or more R⁸; or R⁶ and R² together with the atoms to which they are attached form a 5- to 7-member heterocyclyl optionally substituted with one or more R⁸ groups;
each R⁷ and R⁸ is independently halogen, cyano, nitro, C₁-C₆ alkyl, C₂-C₆ alkenyl, C₁-C₆haloalkyl, C₁-C₆ hydroxyalkyl, —OR¹³, —SR¹³, —N(R¹³)2, —C(O)R¹³, —C(O)OR¹³, —C(O)N(R¹³)₂, or —S(O)₂R¹³, wherein each R¹³ is independently hydrogen or C₁-C₆ alkyl.

31. The method according to claim 30, wherein the dihydrochalcone compound is phloretin.

32. The method according to any of claims 22 to 31, further comprising isolating the product compound from the culture medium.
